SEQUENCE LISTING (207 sequences)

SEQ ID NO: 1 - protein, 124 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
GFTFRDYA

SEQ ID NO: 4 - protein, 8 aa, synthetic construct
ISGSGGNT

SEQ ID NO: 5 - protein, 18 aa, synthetic construct
AKDRLSITIRPRYYGLDV

SEQ ID NO: 6 - protein, 11 aa, synthetic construct
QSLLYSIGYNY

SEQ ID NO: 7 - skipped

SEQ ID NO: 8 - protein, 9 aa, synthetic construct
MQALQTPYT

SEQ ID NO: 9 - protein, 452 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 219 aa, synthetic construct
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 207 aa, synthetic construct
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 12 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 13 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 14 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 15 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 16 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 17 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 18 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 19 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 20 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 21 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 22 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 23 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 24 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 25 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 26 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 27 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 28 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 29 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 30 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 31 - protein, 109 aa, synthetic construct
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 32 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 35 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 36 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 37 - protein, 115 aa, synthetic construct
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, synthetic construct
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 39 - protein, 115 aa, synthetic construct
EVQLVQSGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 40 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 41 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 44 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 45 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 46 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 47 - protein, 115 aa, synthetic construct
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, synthetic construct
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, synthetic construct
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 50 - protein, 115 aa, synthetic construct
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 51 - protein, 115 aa, synthetic construct
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 52 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 53 - protein, 115 aa, synthetic construct
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 54 - protein, 115 aa, synthetic construct
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, synthetic construct
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 56 - protein, 115 aa, synthetic construct
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 57 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 58 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 59 - protein, 115 aa, synthetic construct
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, synthetic construct
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, synthetic construct
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 62 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 63 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 64 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 65 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 66 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 67 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 68 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 69 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYNWGKGTLVTVSS

SEQ ID NO: 70 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 71 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 72 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 73 - protein, 116 aa, synthetic construct; identical to SEQ ID NO: 69

SEQ ID NO: 74 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 75 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 76 - protein, 111 aa, synthetic construct; identical to SEQ ID NO: 74

SEQ ID NO: 77 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 78 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 79 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 80 - protein, 111 aa, synthetic construct; identical to SEQ ID NO: 74

SEQ ID NO: 81 - protein, 116 aa, synthetic construct; identical to SEQ ID NO: 79

SEQ ID NO: 82 - protein, 111 aa, synthetic construct; identical to SEQ ID NO: 78

SEQ ID NO: 83 - protein, 116 aa, synthetic construct; identical to SEQ ID NO: 69

SEQ ID NO: 84 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 85 - protein, 116 aa, synthetic construct; identical to SEQ ID NO: 75

SEQ ID NO: 86 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 87 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 88 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 89 - protein, 116 aa, synthetic construct; identical to SEQ ID NO: 87

SEQ ID NO: 90 - protein, 111 aa, synthetic construct; identical to SEQ ID NO: 84

SEQ ID NO: 91 - protein, 116 aa, synthetic construct; identical to SEQ ID NO: 75

SEQ ID NO: 92 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 93 - protein, 116 aa, synthetic construct; identical to SEQ ID NO: 69

SEQ ID NO: 94 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 95 - protein, 116 aa, synthetic construct; identical to SEQ ID NO: 69

SEQ ID NO: 96 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 97 - protein, 116 aa, synthetic construct; identical to SEQ ID NO: 69

SEQ ID NO: 98 - protein, 111 aa, synthetic construct; identical to SEQ ID NO: 78

SEQ ID NO: 99 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 100 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 101 - protein, 116 aa, synthetic construct; identical to SEQ ID NO: 99

SEQ ID NO: 102 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 103 - protein, 116 aa, synthetic construct; identical to SEQ ID NO: 99

SEQ ID NO: 104 - protein, 111 aa, synthetic construct; identical to SEQ ID NO: 78

SEQ ID NO: 105 - protein, 116 aa, synthetic construct; identical to SEQ ID NO: 77

SEQ ID NO: 106 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 107 - protein, 116 aa, synthetic construct
QVQLVQSGAEVKKPGASVKVSCKASGYAFTSYYMHWARQAPGQGLEWMGIINPSGGSASYAQKFQGRVTMTRDTSTSTVYMELSSLRSEDTAVYYCARGKYWMYDWGKGTLVTVSS

SEQ ID NO: 108 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 109 - protein, 116 aa, synthetic construct; identical to SEQ ID NO: 77

SEQ ID NO: 110 - protein, 111 aa, synthetic construct
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 111 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 112 - protein, 111 aa, synthetic construct
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 113 - protein, 116 aa, synthetic construct
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 114 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 115 - protein, 116 aa, synthetic construct
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 116 - protein, 111 aa, synthetic construct
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 117 - protein, 116 aa, synthetic construct; identical to SEQ ID NO: 77

SEQ ID NO: 118 - protein, 111 aa, synthetic construct; identical to SEQ ID NO: 114

SEQ ID NO: 119 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 120 - protein, 111 aa, synthetic construct
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 121 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 122 - protein, 111 aa, synthetic construct
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 123 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 124 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 125 - protein, 116 aa, synthetic construct; identical to SEQ ID NO: 77

SEQ ID NO: 126 - protein, 111 aa, synthetic construct
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 127 - protein, 116 aa, synthetic construct; identical to SEQ ID NO: 77

SEQ ID NO: 128 - protein, 111 aa, synthetic construct
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 129 - protein, 116 aa, synthetic construct; identical to SEQ ID NO: 77

SEQ ID NO: 130 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 131 - protein, 116 aa, synthetic construct
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 132 - protein, 111 aa, synthetic construct
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 133 - protein, 116 aa, synthetic construct
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 134 - protein, 111 aa, synthetic construct; identical to SEQ ID NO: 114

SEQ ID NO: 135 - protein, 116 aa, synthetic construct; identical to SEQ ID NO: 77

SEQ ID NO: 136 - protein, 111 aa, synthetic construct; identical to SEQ ID NO: 124

SEQ ID NO: 137 - protein, 116 aa, synthetic construct; identical to SEQ ID NO: 107

SEQ ID NO: 138 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDSSTVWEWPFGTGTKLTVL

SEQ ID NO: 139 - protein, 116 aa, synthetic construct
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 140 - protein, 111 aa, synthetic construct
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 141 - protein, 116 aa, synthetic construct; identical to SEQ ID NO: 107

SEQ ID NO: 142 - protein, 111 aa, synthetic construct; identical to SEQ ID NO: 114

SEQ ID NO: 143 - protein, 116 aa, synthetic construct; identical to SEQ ID NO: 113

SEQ ID NO: 144 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 145 - protein, 116 aa, synthetic construct; identical to SEQ ID NO: 77

SEQ ID NO: 146 - protein, 111 aa, synthetic construct; identical to SEQ ID NO: 138

SEQ ID NO: 147 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 148 - protein, 111 aa, synthetic construct
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 149 - protein, 116 aa, synthetic construct
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 150 - protein, 111 aa, synthetic construct
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 151 - protein, 116 aa, synthetic construct
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 152 - protein, 111 aa, synthetic construct
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 153 - protein, 116 aa, synthetic construct
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 154 - protein, 121 aa, synthetic construct
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 155 - protein, 127 aa, synthetic construct
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 156 - protein, 116 aa, synthetic construct
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 157 - protein, 116 aa, synthetic construct
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 158 - protein, 116 aa, synthetic construct
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 159 - protein, 116 aa, synthetic construct
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 160 - protein, 116 aa, synthetic construct
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 161 - protein, 116 aa, synthetic construct
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 162 - protein, 116 aa, synthetic construct
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 163 - protein, 116 aa, synthetic construct
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 164 - protein, 116 aa, synthetic construct
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 165 - protein, 116 aa, synthetic construct
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 166 - protein, 116 aa, synthetic construct
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 167 - protein, 116 aa, synthetic construct
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 168 - protein, 110 aa, synthetic construct
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 169 - protein, 110 aa, synthetic construct
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 170 - protein, 110 aa, synthetic construct
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 171 - protein, 110 aa, synthetic construct
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 172 - protein, 110 aa, synthetic construct
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 173 - protein, 110 aa, synthetic construct
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 174 - protein, 110 aa, synthetic construct
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 175 - protein, 110 aa, synthetic construct
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 176 - protein, 110 aa, synthetic construct
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 177 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 178 - protein, 110 aa, synthetic construct; identical to SEQ ID NO: 177

SEQ ID NO: 179 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 180 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 181 - protein, 110 aa, synthetic construct; identical to SEQ ID NO: 177

SEQ ID NO: 182 - protein, 111 aa, synthetic construct
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 183 - protein, 111 aa, synthetic construct
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 184 - protein, 111 aa, synthetic construct
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 185 - protein, 111 aa, synthetic construct
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 186 - protein, 111 aa, synthetic construct
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 187 - protein, 111 aa, synthetic construct
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 188 - protein, 117 aa, synthetic construct
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 189 - protein, 128 aa, synthetic construct
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 190 - protein, 122 aa, synthetic construct
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 191 - protein, 128 aa, synthetic construct
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 192 - protein, 117 aa, synthetic construct
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 193 - protein, 124 aa, synthetic construct
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 194 - protein, 117 aa, synthetic construct
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 195 - protein, 116 aa, synthetic construct
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 196 - protein, 117 aa, synthetic construct
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 197 - protein, 122 aa, synthetic construct
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 198 - protein, 107 aa, synthetic construct
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 199 - protein, 107 aa, synthetic construct
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 200 - protein, 107 aa, synthetic construct
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 201 - protein, 107 aa, synthetic construct
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 202 - protein, 107 aa, synthetic construct
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 203 - protein, 107 aa, synthetic construct
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 204 - protein, 107 aa, synthetic construct
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 205 - protein, 107 aa, synthetic construct
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 206 - protein, 107 aa, synthetic construct
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 207 - protein, 107 aa, synthetic construct
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK

(19) United States
(12) Patent Application Publication
(10) Pub. No.: US 2025/0145662 A1
(43) Pub. Date: May 8, 2025

(54) METHODS FOR REDUCING LIPASE ACTIVITY USING STRESS

(71) Applicant: Regeneron Pharmaceuticals, Inc., Tarrytown, NY (US)

(72) Inventors: Xiaolin Tang, Old Tappan, NJ (US); Leonid Breydo, Bronx, NY (US)

(21) Appl. No.: 18/934,626

(22) Filed: Nov. 1, 2024

Related U.S. Application Data
(60) Provisional application No. 63/547,123, filed on Nov. 2, 2023.

(51) Int. Cl. C07K 1/18; C07K 1/36; C07K 16/28

(52) U.S. Cl. CPC C07K 1/18; C07K 1/36; C07K 16/2866

(57) ABSTRACT

Pharmaceutical formulations comprising an antibody that specifically binds to human interleukin-4 receptor alpha (hIL-4Rα) are provided. The formulations may contain, in addition to an anti-IL-4Rα antibody, one or more buffers, at least one amino acid, at least one sugar, and a surfactant. Methods for producing pharmaceutical formulations with reduced lipase activity are also provided, which may include subjecting a drug substance to agitation stress, heat stress, and additional ion exchange or size exclusion chromatography. In one aspect, the pharmaceutical formulations do not have appreciable subvisible particle formation in the presence of lipase, and exhibit a substantial degree of antibody stability during storage and after being subjected to thermal and other physical stresses.

### CROSS-REFERENCE TO REALTED APPLICATIONS

This application claims the benefit of U.S. Provisional Patent Application No. 63/547,123, filed on Nov. 2, 2023, which is incorporated herein by reference in its entirety.

### SEQUENCE LISTING

The instant application contains a Sequence Listing which has been submitted electronically in XML format and is hereby incorporated by reference in its entirety. Said XML copy, created on Jul. 11, 2023, is named 20230713-04850.xml and is 201,551 bytes in size.

### BACKGROUND

Among drug products, protein-based biotherapeutics are an important class of drugs that offer a high level of selectivity, potency and efficacy, as evidenced by the considerable increase in clinical trials with monoclonal antibodies (mAbs) over the past several years. One critical aspect for a clinically and commercially viable biotherapeutic is stability of the drug product in terms of the manufacturing process, as well as shelf-life. Surfactants, such as polysorbate, are often used to enhance the physical stability of a protein-based biotherapeutics product. Over seventy percent of marketed monoclonal antibody therapeutics contain between 0.001% and 0.2% polysorbate, a type of surfactant, to impart physical stability to the protein-based biotherapeutics.

Enzymatic hydrolysis of polysorbate has been recognized as the primary route of polysorbate degradation in biotherapeutics formulations. Polysorbate degradation may be caused by co-purification of esterases or lipases during production of a drug product. Polysorbate hydrolysis results in the release of free fatty acids that can drive undesirable particulate formation in a drug product. Particles may be visible or subvisible; subvisible particles typically are under 150 microns or 100 microns in diameter.

Thus, a need exists for drug products with reduced esterase or lipase activity and reduced formation of free fatty acid particles, and for methods for making the same.

### SUMMARY

Products and methods have been developed for reducing esterase activity in a composition or formulation, reducing lipase activity in a composition or formulation, reducing polysorbate degradation, and reducing fatty acid particle formation. In one aspect, a formulated drug substance comprising a protein of interest, a fatty acid ester and an esterase or lipase is subjected to stress, for example agitation stress or heat stress, to inactivate the esterase or lipase, degrade the esterase or lipase, and/or reduce esterase or lipase activity. These may cause the formation of high molecular weight (HMW) species of esterase or lipase and/or drug protein. The substance may then be subjected to filtration, enrichment, and/or separation, for example using cation exchange chromatography or size exclusion chromatography, to remove HMW species and produce a formulated drug substance and/or drug product with reduced esterase or lipase activity, reduced polysorbate degradation, and/or reduced free fatty acid particle formation compared to a sample that was not subjected to stress and/or filtration/separation. In some aspects, the protein of interest is an anti-IL4Rα antibody. In some aspects, the anti-IL-4Rα antibody is Dupilumab.

This disclosure provides methods for producing a pharmaceutical composition with reduced lipase activity, improved fatty acid ester stability, reduced fatty acid ester degradation, and/or reduced fatty acid particle formation. In some exemplary aspects, these methods can comprise: (a) subjecting a sample including a protein of interest and a lipase to stress conditions to form a sample with inactivated lipase; (b) subjecting said sample with inactivated lipase to a purification step to form a HMW-depleted sample; and (c) formulating said HMW-depleted sample with at least one fatty acid ester to produce a pharmaceutical composition with reduced lipase activity, improved polysorbate stability, reduced polysorbate degradation, and/or reduced fatty acid particle formation compared to a pharmaceutical composition formulated from a sample that was not subjected to stress conditions, and wherein said reduced lipase activity, improved polysorbate stability, reduced polysorbate degradation, and/or reduced fatty acid particle formation is measured after a period of storage at long-term storage conditions or accelerated storage conditions.

In one aspect, the protein of interest is an antibody, an antibody-derived protein, an antibody fragment, a monoclonal antibody, a bispecific antibody, a fusion protein, an antibody-drug conjugate, or a therapeutic protein. In a specific aspect, the protein of interest is an anti-IL4Rα antibody. In another specific aspect, the protein of interest is Dupilumab.

In one aspect, the stress conditions include agitation stress and/or heat stress. In a specific aspect, the agitation stress comprises shaking said sample at from 50 to 500 rpm, from 200 to 300 rpm, about 50 rpm, about 75 rpm, about 100 rpm, about 125 rpm, about 150 rpm, about 200 rpm, about 225 rpm, about 250 rpm, about 275 rpm, about 300 rpm, about 325 rpm, about 350 rpm, about 375 rpm, about 400 rpm, about 425 rpm, about 450 rpm, about 475 rpm, or about 500 rpm.

In another specific aspect, the agitation stress comprises shaking said sample for from 1 to 96 hours, from 24 to 48 hours, about 1 hour, about 2 hours, about 3 hours, about 4 hours, about 5 hours, about 6 hours, about 12 hours, about 18 hours, about 24 hours, about 30 hours, about 36 hours, about 42 hours, about 48 hours, about 60 hours, about 72 hours, about 84 hours, or about 96 hours.

In an additional specific aspect, the heat stress comprises storing said sample at from about 25° C. to about 60° C., from about 30° C. to about 60° C., from about 35° C. to about 55° C., from about 40° C. to about 50° C., from about 44° C. to about 46° C., about 25° C., about 30° C., about 35° C., about 40° C., about 45° C., about 50° C., about 55° C., or about 60° C.

In a more specific aspect, the heat stress comprises storing said sample at said temperature from 1 day to 6 months, from 3 days to 3 months, from 1 week to 2 months, from 0.5 months to 1 month, about 1 day, about 2 days, about 3 days, about 1 week, about 2 weeks, about 0.5 months, about 3 weeks, about 4 weeks, about 1 month, about 5 weeks, about 6 weeks, about 7 weeks, about 8 weeks, about 2 months, about 3 months, about 3.5 months, about 4 months, about 4.5 months, about 5 months, about 5.5 months, or about 6 months.

In one aspect, the purification step comprises filtration, enrichment, or chromatographic separation. In a specific aspect, the chromatographic separation comprises cation exchange chromatography. In another specific aspect, the chromatographic separation comprises size exclusion chromatography. In an additional specific aspect, the chromatographic separation comprises anion exchange chromatography. In a further specific aspect, the chromatographic separation comprises hydrophobic interaction chromatography.

In one aspect, the purification step is selected to remove HMW species. In another aspect, the purification step substantially removes HMW species.

In one aspect, the HMW-depleted sample comprises less than 10%, less than 5%, less than 4%, less than 3%, less than 2%, or less than 1% HMW species. In another aspect, the HMW-depleted sample comprises less than 5% HMW species. In a further aspect, the HMW-depleted sample comprises a percentage of HMW species that is about the same as the percentage of HMW species in a sample that was not subjected to stress conditions and a purification step.

In one aspect, the formulating step further comprises adding excipients to the HMW-depleted sample.

In one aspect, the fatty acid ester is a polysorbate. In a specific aspect, the polysorbate is polysorbate 20, polysorbate 40, polysorbate 60, or polysorbate 80.

In one aspect, the period of storage is from about 2 weeks to about 5 years, from about 2 weeks to about 3 years, from about 2 weeks to about 1 year, from about 2 weeks to about 6 months, from about 1 month to about 5 years, from about 1 month to about 3 years, from about 1 month to about 1 year, from about 1 month to about 6 months, about 2 weeks, about 3 weeks, about 4 weeks, about 1 month, about 2 months, about 3 months, about 4 months, about 5 months, about 6 months, about 9 months, about 12 months, about 18 months, about 2 years, about 3 years, about 4 years, or about 5 years.

In one aspect, the long-term storage conditions comprise storage at a temperature from about 0° C. to about 10° C., about 0° C., about 5° C., or about 10° C. In another aspect, the accelerated storage conditions comprise storage at a temperature from about 35° C. to about 60° C., from about 45° C. to about 55° C., about 35° C., about 40° C., about 45° C., about 50° C., about 55° C., or about 60° C.

In one aspect, a measure of reduced lipase activity and/or fatty acid ester stability comprises a quantification of fatty acid ester degradation. In a specific aspect, the quantification comprises an enzymatic colorimetric assay for non-esterified fatty acids, mass spectrometry, charged aerosol detection, and/or liquid chromatography. In another specific aspect, the quantification comprises determining a percent of fatty acid ester recovered from said pharmaceutical composition with reduced lipase activity after said period of storage using charged aerosol detection-liquid chromatography. In a more specific aspect, the percent is above 80%, above 85%, above 90%, above 95%, above 99%, about 85%, about 90%, about 95%, about 99%, or about 100%.

In one aspect, a measure of reduced lipase activity, improved fatty acid ester stability, reduced fatty acid ester degradation, and/or reduced fatty acid particle formation comprises a quantification of particles per unit volume. In a specific aspect, the particles comprise visible particles and/or subvisible particles. In another specific aspect, the particles comprise particles≥10 um in diameter and/or particles≥25 um in diameter. In an additional specific aspect, the particles comprise fatty acid particles. In a further specific aspect, the quantification comprises light obscuration, optical microscopy, micro-flow image analysis, flow cytometry, Coulter counting, and/or submicron particle tracking. In another specific aspect, a number of particles per mL in said pharmaceutical composition with reduced lipase activity is below 500, below 200, below 100, below 50, below 10, or below 5.

In one aspect, the protein of interest is produced from a recombinant host cell. In a specific aspect, the recombinant host cell is selected from a group consisting of a CHO cell, a CHO-K1 cell, and variations thereof.

In one aspect, the lipase is a host cell protein. In another aspect, the lipase has an increased susceptibility to inactivation in stress conditions compared to said protein of interest.

This disclosure further provides a protein formulation comprising a protein and at least one fatty acid ester made by the method of any of the above aspects or embodiments.

These, and other, aspects of the invention will be better appreciated and understood when considered in conjunction with the following description and accompanying drawings. The following description, while indicating various aspects and numerous specific details thereof, is given by way of illustration and not of limitation. Many substitutions, modifications, additions, or rearrangements may be made within the scope of the invention.

### DETAILED DESCRIPTION

Therapeutic macromolecules must be formulated in a manner that not only makes the molecules suitable for administration to patients, but also maintains their stability during storage. For example, therapeutic antibodies in liquid solution are prone to degradation, aggregation and/or undesired chemical modifications unless the solution is formulated properly. The stability of an antibody in liquid formulation depends not only on the kinds of excipients used in the formulation, but also on the amounts and proportions of the excipients relative to one another. Therapeutic formulations also may be subject to the formation of particulate matter over time during storage. Particles may be visible or subvisible; subvisible particles typically are under 150 microns or 100 microns in diameter. Formulations having high protein concentrations, e.g., concentrations of 30 mg/mL or higher, are more prone to aggregation and subvisible particle formation.

Polysorbate 20 (PS20) and polysorbate 80 (PS80) are the most commonly used nonionic surfactants in biopharmaceutical protein formulations for improving protein stability and protecting protein products from aggregation and denaturation (Martos et al., J Pharm Sci. 106 (7): 1722-1735 (2017); Kiese et al., J Pharm Sci 97 (19): 4347-4366 (2008); Dwivedi et al., Int J Pharm 552 (1-2): 422-436 (2018)). However, it has been reported that polysorbates, including polysorbate 20 and polysorbate 80, can degrade in the presence of lipase, which over time results in the formation of subvisible particles in a formulation.

Polysorbates (PSs) are known to be liable to degradation via two main pathways: autooxidation and hydrolysis (Dwivedi et al.; Kishore et al., Pharm Res. 28 (5): 1194-1210 (2011); Larson et al., J Pharm Sci. 109 (10): 633-639 (2020); Kishore et al. J Pharm Sci. 100 (2): 721-731 (2011)). Enzymatic hydrolysis is considered to be the primary route of PS degradation in high-concentration protein formulations, which results in the accumulation of free fatty acids (FFAs) that may drive undesirable particulate formation in the drug products. Oxidation is the second primary pathway for PS degradation, which leads to the formation of peroxides, aldehydes, ketones and short-chain esterified POE sorbitan/isosorbide species (Kishore et al. 2011a; Larson et al.; Kishore et al. 2011b; Donbrow et al., J Pharm Sci. 67 (12): 1676-1681 (1978); Yao et al. Pharm Res. 26 (10): 2303-2313 (2009)). PS hydrolysis has been recognized as a larger threat to drug product quality because this process not only reduces the PS concentration, but is also associated with particulate formation due to the low solubility of accumulated FFAs, especially at storage temperatures of 2° C.-8° C. (Doshi et al., J Pharm Sci. 110 (2): 687-692 (2021); Saggu et al., J Pharm Sci. 110 (3): 1093-1102 (2021); Doshi et al. Mol Pharm. 12 (11): 3792-3804 (2015)).

Residual lipases or esterase present in drug product are the major cause of PS hydrolysis (Chiu et al., Biotechnol Bioeng. 114 (5): 1006-1015 (2017); Hall et al. J Pharm Sci. 105 (5): 1633-1642 (2016); Labrenz et al. J Pharm Sci. 103 (8): 2268-2277 (2014); McShan et al. PDA J Pharm Sci Technol. 70 (4): 332-345 (2016); Zhang et al. J Pharm Sci. 109 (11): 3300-3307 (2020); Zhang et al. J Pharm Sci. 109 (9): 2710-2718 (2020)). See also U.S. patent application Ser. No. 18/310,921, the entire teaching of which is herein incorporated by reference. The levels of residual lipases in final drug product are usually very low (<10 ppm) after multiple steps of downstream purification, and the consequences of PS degradation may not be noticeable until after storage for months or years at typical storage temperatures (2° C.-8° C.).

Without intending to be bound by theory, it is believed that a putative phospholipase B-like 2 (PLBL2), which is highly conserved in hamster, rat, mice, human and bovine, copurifies with some classes of proteins under certain processes. Other esterases or lipases may also copurify with proteins of interest at concentrations too low to be reliably detected but high enough to have measurable lipase activity, eventually resulting in a loss of polysorbate, production of free fatty acids, and the formation of visible or sub-visible particles. Therefore, a need exists for drug products with reduced lipase activity, reduced esterase activity, and reduced formation of fatty acid particles, and for methods for making the same.

Disclosed herein are novel drug products, compositions and formulations with reduced lipase activity, reduced esterase activity, and reduced formation of fatty acid particles, and methods for making the same. The inventors have surprisingly discovered that co-purified esterases or lipases can be successfully inactivated and/or removed by the application of stress, for example agitation stress or heat stress, which can cause esterases or lipases to degrade, reducing lipase activity. The application of stress may also lead to the formation of high molecular weight (HMW) species. The HMW species can be removed from the drug substance using, for example, molecular weight filtration or chromatography, such as cation exchange chromatography or size exclusion chromatography. These, and other aspects of the invention are set forth in further detail below.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, bispecific antibodies, and antigen-binding proteins.

Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated by reference). In some aspects, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain aspects, the recombinant protein can be an antibody, for example, a chimeric, humanized, or fully human antibody. In certain aspects, the recombinant protein can be an antibody of an isotype selected from group consisting of: IgG, IgM, IgA1, IgA2, IgD, or IgE. In certain aspects the antibody molecule is a full-length antibody (e.g., an IgG1) or alternatively the antibody can be a fragment (e.g., an Fc fragment or a Fab fragment).

### Antibodies

The term “antibody,” as used herein, is generally intended to refer to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody.” Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementary determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

In some aspects, the protein of interest is a human antibody. The term “human antibody,” as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

Interleukin-4 (IL-4) and interleukin-13 (IL-13) are key cytokines in driving allergic and T helper cell type 2 (Th2) polarized inflammatory processes. IL-4 and IL-13 signaling is mediated through heterodimeric receptor complexes, in which IL-4 receptor alpha (IL-4Rα) is a shared receptor subunit for both IL-4 and IL-13 signaling. Thus, IL-4Rα is an attractive therapeutic target because it provides a single target for blocking both IL-4 and IL-13 signaling. In some aspects, the protein of interest is an anti-IL-4R antibody, or an antigen-binding fragment thereof. Antibodies to hIL-4Rα are described in, for example, U.S. Pat. Nos. 5,717,072, 7,186,809 and 7,605,237.

In some aspects, the anti-IL-4R antibody is a human IgG antibody. In various aspects, the anti-IL-4R antibody is a human antibody of isotype IgG1, IgG2, IgG3 or IgG4, or mixed isotype. In some aspects, the anti-IL-4R antibody is a human IgG1 antibody. In some aspects, the anti-IL-4R antibody is a human IgG4 antibody. In any of the aspects discussed above or herein, the anti-IL-4R antibody may comprise a human kappa light chain. In any of the aspects discussed above or herein, the anti-IL-4R antibody may comprise a human lambda light chain.

The antibodies of the disclosure may, in some aspects, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain aspects, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some aspects, an antibody fragment comprises a sufficient amino acid sequence of the parent antibody of which it is a fragment that it binds to the same antigen as does the parent antibody; in some aspects, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or KA-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

An “isolated antibody,” as used herein, is intended to refer to an antibody that is substantially free of other antibodies having different antigenic specificities (e.g., an isolated antibody that specifically binds hIL-4Rα is substantially free of antibodies that specifically bind antigens other than hIL-4Rα).

The term “specifically binds,” or the like, means that an antibody or antigen-binding fragment thereof forms a complex with an antigen that is relatively stable under physiologic conditions. Specific binding can be characterized by a dissociation constant of at least about 1×10⁻⁶ M or greater. Methods for determining whether two molecules specifically bind are well known in the art and include, for example, equilibrium dialysis, surface plasmon resonance, and the like. An isolated antibody that specifically binds hIL-4Rα may, however, have cross-reactivity to other antigens, such as IL-4Rα molecules from other species (orthologs). In the context of the present disclosure, multispecific (e.g., bispecific) antibodies that bind to hIL-4Rα as well as one or more additional antigens are deemed to “specifically bind” hIL-4Rα. Moreover, an isolated antibody may be substantially free of other cellular material and/or chemicals. However, in some instances, the isolated antibody may be copurified with a phospholipase expressed by a mammalian cell line (e.g., CHO cells) from which the anti-IL-4R antibody is produced.

For example, for antibody production, aspects of the inventions are amenable for research and production use for diagnostics and therapeutics based on all major antibody classes, namely IgG, IgA, IgM, IgD, and IgE. IgG is a preferred class, and includes subclasses IgG1 (including IgG1λ and IgG1κ), IgG2, IgG3, and IgG4. In some aspects, the protein of interest or polypeptide of interest is an antibody, a human antibody, a humanized antibody, a chimeric antibody, a monoclonal antibody, a multispecific antibody, a bispecific antibody, an antibody fragment, an antigen-binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, an IgG4 antibody, a fusion protein, a receptor fusion protein, an antibody-derived protein, or combinations thereof. In one aspect, the antibody is an IgG1 antibody. In one aspect, the antibody is an IgG2 antibody. In one aspect, the antibody is an IgG4 antibody. In one aspect, the antibody is a chimeric IgG2/IgG4 antibody. In one aspect, the antibody is a chimeric IgG2/IgG1 antibody. In one aspect, the antibody is a chimeric IgG2/IgG1/IgG4 antibody. Derivatives, components, domains, chains, and fragments of the above are also included.

In some aspects, the antibody is selected from the group consisting of an anti-Programmed Cell Death 1 antibody (e.g. an anti-PD1 antibody as described in U.S. Pat. App. Pub. No. US2015/0203579A1), an anti-Programmed Cell Death Ligand-1 antibody (e.g. an anti-PD-L1 antibody as described in in U.S. Pat. App. Pub. No. US2015/0203580A1), an anti-DII4 antibody, an anti-Angiopoietin-2 antibody (e.g. an anti-ANG2 antibody as described in U.S. Pat. No. 9,402,898), an anti-Angiopoietin-Like 3 antibody (e.g. an anti-AngPt13 antibody as described in U.S. Pat. No. 9,018,356), an anti-platelet derived growth factor receptor antibody (e.g. an anti-PDGFR antibody as described in U.S. Pat. No. 9,265,827), an anti-Erb3 antibody, an anti-Prolactin Receptor antibody (e.g. anti-PRLR antibody as described in U.S. Pat. No. 9,302,015), an anti-Complement 5 antibody (e.g. an anti-C5 antibody as described in U.S. Pat. App. Pub. No US2015/0313194A1), an anti-TNF antibody, an anti-epidermal growth factor receptor antibody (e.g. an anti-EGFR antibody as described in U.S. Pat. No. 9,132,192 or an anti-EGFRvIII antibody as described in U.S. Pat. App. Pub. No. US2015/0259423A1), an anti-Proprotein Convertase Subtilisin Kexin-9 antibody (e.g. an anti-PCSK9 antibody as described in U.S. Pat. No. 8,062,640 or U.S. Pat. App. Pub. No. US2014/0044730A1), an anti-Growth And Differentiation Factor-8 antibody (e.g. an anti-GDF8 antibody, also known as anti-myostatin antibody, as described in U.S. Pat. No. 8,871,209 or U.S. Pat. No. 9,260,515), an anti-Glucagon Receptor (e.g. anti-GCGR antibody as described in U.S. Pat. App. Pub. Nos. US2015/0337045A1 or US2016/0075778A1), an anti-VEGF antibody, an anti-IL1R antibody, an interleukin 4 receptor antibody (e.g., an anti-IL4R antibody as described in U.S. Pat. App. Pub. No. US2014/0271681A1 or U.S. Pat. No. 8,735,095 or U.S. Pat. No. 8,945,559), an anti-interleukin 6 receptor antibody (e.g. an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (e.g. anti-IL33 antibody as described in U.S. Pat. App. Pub. Nos. US2014/0271658A1 or US2014/0271642A1), an anti-Cluster of differentiation 3 antibody (e.g. an anti-CD3 antibody, as described in U.S. Pat. App. Pub. Nos. US2014/0088295A1 and US20150266966A1, and in U.S. Application No. 62/222,605), an anti-Cluster of differentiation 20 antibody (e.g. an anti-CD20 antibody as described in U.S. Pat. App. Pub. Nos. US2014/0088295A1 and US20150266966A1, and in U.S. Pat. No. 7,879,984), an anti-CD19 antibody, an anti-CD28 antibody, an anti-Cluster of Differentiation-48 antibody (e.g. anti-CD48 antibody as described in U.S. Pat. No. 9,228,014), an anti-Fel d1 antibody (e.g. as described in U.S. Pat. No. 9,079,948), an anti-influenza virus antibody, an anti-Respiratory syncytial virus antibody (e.g. anti-RSV antibody as described in U.S. Pat. App. Pub. No. US2014/0271653A1), an anti-Middle East Respiratory Syndrome virus antibody (e.g. an anti-MERS-CoV antibody as described in U.S. Pat. App. Pub. No. US2015/0337029A1), an anti-Ebola virus antibody (e.g. as described in U.S. Pat. App. Pub. No. US2016/0215040), an anti-Zika virus antibody, an anti-Severe Acute Respiratory Syndrome (SARS) antibody (e.g., an anti-SARS-CoV antibody), an anti-COVID-19 antibody (e.g., an anti-SARS-CoV-2 antibody), an anti-Lymphocyte Activation Gene 3 antibody (e.g. an anti-LAG3 antibody, or an anti-CD223 antibody), an anti-Nerve Growth Factor antibody (e.g. an anti-NGF antibody as described in U.S. Pat. App. Pub. No. US2016/0017029 and U.S. Pat. Nos. 8,309,088 and 9,353,176) and an anti-Activin A antibody. In some aspects, the bispecific antibody is selected from the group consisting of an anti-CD3×anti-CD20 bispecific antibody (as described in U.S. Pat. App. Pub. Nos. US2014/0088295A1 and US20150266966A1), an anti-CD3×anti-Mucin 16 bispecific antibody (e.g., an anti-CD3×anti-Muc16 bispecific antibody), an anti-CD3×BCMA bispecific antibody, and an anti-CD3×anti-Prostate-specific membrane antigen bispecific antibody (e.g., an anti-CD3×anti-PSMA bispecific antibody). See also U.S. Patent Publication No. US 2019/0285580 A1. Also included are a Met×Met antibody, an agonist antibody to NPR1, an LEPR agonist antibody, a MUC16×CD28 antibody, a GITR antibody, an IL-2Rg antibody, an EGFR×CD28 antibody, a Factor XI antibody, antibodies against SARS-CoV-2 variants, a Fel d 1 multi-antibody therapy, and a Bet v 1 multi-antibody therapy. Derivatives, components, domains, chains and fragments of the above also are included. In one aspect, the protein of interest or polypeptide of interest comprises a combination of any of the foregoing.

Cells that produce exemplary antibodies can be cultured according to the inventions. In some aspects, the protein of interest or polypeptide of interest is selected from the group consisting of Alirocumab, Atoltivimab, Maftivimab, Odesivimab, Odesivimab-ebgn, Casirivimab, Imdevimab, Cemplimab and Cemplimab-rwlc (human IgG4 monoclonal antibody that binds to PD-1), Sarilumab, Fasinumab, Nesvacumab, Dupilumab (human monoclonal antibody of the IgG4 subclass that binds to the IL-4R alpha (α) subunit and thereby inhibits Interleukin 4 (IL-4) and Interleukin 13 (IL-13) signaling), Trevogrumab, Evinacumab, Evinacumab-dgnb, Fianlimab, Garetosmab, Itepekimab, Odrononextamab, Pozelimab, Rinucumab, and modifications, truncations, and variations thereof.

Additional exemplary antibodies include Ravulizumab-cwvz, Abciximab, Adalimumab, Adalimumab-atto, Ado-trastuzumab, Alemtuzumab, Atezolizumab, Avelumab, Basiliximab, Belimumab, Benralizumab, Bevacizumab, Bezlotoxumab, Blinatumomab, Brentuximab vedotin, Brodalumab, Canakinumab, Capromab pendetide, Certolizumab pegol, Cetuximab, Denosumab, Dinutuximab, Durvalumab, Eculizumab, Elotuzumab, Emicizumab-kxwh, Emtansine alirocumab, Evolocumab, Golimumab, Guselkumab, Ibritumomab tiuxetan, Idarucizumab, Infliximab, Infliximab-abda, Infliximab-dyyb, Ipilimumab, Ixekizumab, Mepolizumab, Necitumumab, Nivolumab, Obiltoxaximab, Obinutuzumab, Ocrelizumab, Ofatumumab, Olaratumab, Omalizumab, Panitumumab, Pembrolizumab, Pertuzumab, Ramucirumab, Ranibizumab, Raxibacumab, Reslizumab, Rinucumab, Rituximab, Secukinumab, Siltuximab, Tocilizumab, Trastuzumab, Ustekinumab, and Vedolizumab.

In addition to next generation products, the inventions also are applicable to production of biosimilars. Biosimilars are defined in various ways depending on the jurisdiction, but share a common feature of comparison to a previously approved biological product in that jurisdiction, usually referred to as a “reference product.” According to the World Health Organization, a biosimilar is a biotherapeutic product similar to an already licensed reference biotherapeutic product in terms of quality, safety and efficacy, and is followed in many countries, such as the Philippines.

A biosimilar in the U.S. is currently described as (A) a biological product that is highly similar to the reference product notwithstanding minor differences in clinically inactive components; and (B) there are no clinically meaningful differences between the biological product and the reference product in terms of the safety, purity, and potency of the product. In the U.S., an interchangeable biosimilar or product may be substituted for the previous product without the intervention of the health care provider who prescribed the previous product. In the European Union, a biosimilar is a biological medicine highly similar to another biological medicine already approved in the EU (called “reference medicine”) and includes consideration of structure, biological activity, efficacy, and safety, among other things, and these guidelines also are followed by Russia. In China, a biosimilar product currently refers to biologics that contain active substances similar to the original biologic drug and is similar to the original drug in terms of quality, safety, and effectiveness, with no clinically significant differences. In Japan, a biosimilar currently is a product that has bioequivalent/quality-equivalent quality, safety, and efficacy to a reference product already approved in Japan. In India, biosimilars currently are referred to as “similar biologics,” and refer to a similar biologic product which is similar in terms of quality, safety, and efficacy to an approved reference biological product based on comparability. In Australia, a biosimilar medicine currently is a highly similar version of a reference biological medicine. In Mexico, Columbia, and Brazil, a biosimilar currently is a biotherapeutic product that is similar in terms of quality, safety, and efficacy to an already licensed reference product. In Argentina, a biosimilar currently is derived from an original product (a comparator) with which it has common features. In Singapore, a biosimilar currently is a biological therapeutic product that is similar to an existing biological product registered in Singapore in terms of physicochemical characteristics, biological activity, safety and efficacy. In Malaysia, a biosimilar currently is a new biological medicinal product developed to be similar in terms of quality, safety and efficacy to an already registered, well established medicinal product. In Canada, a biosimilar currently is a biologic drug that is highly similar to a biologic drug that was already authorized for sale. In South Africa, a biosimilar currently is a biological medicine developed to be similar to a biological medicine already approved for human use. Production of biosimilars and its synonyms under these and any revised definitions can be undertaken according to the inventions.

According to certain aspects of the present disclosure, the anti-hIL-4R antibody, or antigen-binding fragment thereof, comprises heavy chain complementarity determining regions HCDR1-HCDR2-HCDR3, respectively, comprising the amino acid sequences of SEQ ID NOs: 3-4-5. According to certain aspects of the present disclosure, the anti-hIL-4R antibody, or antigen-binding fragment thereof, comprises light chain complementarity determining regions LCDR1-LCDR2-LCDR3, respectively, comprising the amino acid sequences of SEQ ID NOs: 6-7-8. In certain aspects, the anti-hIL-4R antibody, or antigen-binding fragment thereof, comprises the CDRs HCDR1-HCDR2-HCDR3-LCDR1-LCDR2-LCDR3, respectively, comprising the amino acid sequences of SEQ ID NOs: 3-4-5-6-7-8.

In certain aspects, the anti-hIL-4R antibody, or antigen-binding fragment thereof, comprises heavy chain complementarity determining regions HCDR1-HCDR2-HCDR3, respectively, comprising the amino acid sequences of SEQ ID NOs: 3-4-5 and has a heavy chain variable region (HCVR) having at least 90% sequence identity (e.g., at least 91%, 92%, 93%, 94%, 95%, 96%, 97%, 98%, or 99% sequence identity) to the amino acid sequence of SEQ ID NO: 1. In certain aspects, the anti-hIL-4R antibody, or antigen-binding fragment thereof, comprises light chain complementarity determining regions LCDR1-LCDR2-LCDR3, respectively, comprising the amino acid sequences of SEQ ID NOs: 6-7-8 and has a light chain variable region (LCVR) having at least 90% sequence identity (e.g., at least 91%, 92%, 93%, 94%, 95%, 96%, 97%, 98%, or 99% sequence identity) to the amino acid sequence of SEQ ID NO: 2. In certain aspects, the anti-hIL-4R antibody, or antigen-binding fragment thereof, comprises: heavy chain complementarity determining regions HCDR1-HCDR2-HCDR3, respectively, comprising the amino acid sequences of SEQ ID NOs: 3-4-5 and has a heavy chain variable region (HCVR) having at least 90% sequence identity (e.g., at least 91%, 92%, 93%, 94%, 95%, 96%, 97%, 98%, or 99% sequence identity) to the amino acid sequence of SEQ ID NO: 1; and light chain complementarity determining regions LCDR1-LCDR2-LCDR3, respectively, comprising the amino acid sequences of SEQ ID NOs: 6-7-8 and has a light chain variable region (LCVR) having at least 90% sequence identity (e.g., at least 91%, 92%, 93%, 94%, 95%, 96%, 97%, 98%, or 99% sequence identity) to the amino acid sequence of SEQ ID NO: 2.

In certain aspects, the anti-hIL-4R antibody, or antigen-binding fragment thereof, comprises a heavy chain variable region (HCVR) comprising the amino acid sequence of SEQ ID NO: 1. In certain aspects, the anti-hIL-4R antibody, or antigen-binding fragment thereof, comprises a light chain variable region (LCVR) comprising the amino acid sequence of SEQ ID NO: 2. In certain aspects, the anti-hIL-4R antibody, or antigen-binding fragment thereof, comprises a HCVR/LCVR amino acid sequence pair comprising the amino acid sequences of SEQ ID NOs: ½. In some aspects, the anti-IL-4R antibody comprises a HCVR/LCVR comprising the amino acid sequences of SEQ ID NOs: ½, respectively, and a human IgG1 heavy chain constant region. In some aspects, the anti-IL-4R antibody comprises a HCVR/LCVR comprising the amino acid sequences of SEQ ID NOs: ½, respectively, and a human IgG4 heavy chain constant region. In some aspects, the anti-IL-4R antibody comprises a HCVR/LCVR comprising the amino acid sequences of SEQ ID NOs: ½, respectively, and a human IgG heavy chain constant region. In some aspects, the anti-IL-4R antibody comprises a HCVR/LCVR comprising the amino acid sequences of SEQ ID NOs: ½, respectively, and a human IgG1 or IgG4 heavy chain constant region. In some aspects, the anti-IL-4R antibody comprises a heavy chain comprising the amino acid sequence of SEQ ID NO: 9 and a light chain comprising the amino acid sequence of SEQ ID NO: 10. In some aspects, the anti-IL-4R antibody is dupilumab.

Other anti-IL-4R antibodies that can be used in the context of the methods of the present disclosure include, for example, the antibody referred to and known in the art as AMG317 (Corren et al., 2010, Am J Respir Crit Care Med., 181 (8): 788-796), or MEDI 9314, or any of the anti-IL-4Rα antibodies as set forth in U.S. Pat. Nos. 7,186,809, 7,605,237, 7,638,606, 8,092,804, 8,679,487, 8,877,189, 10,774,141; US Patent Application Publication No. US2021/0238294; or International Patent Publication Nos. WO2019/228405, WO2020/096381, WO 2020/135471, WO2020/135710, or WO 2020/239134, the contents of each of which are incorporated by reference herein.

In some aspects, the anti-IL-4R antibody comprises one or more CDR, HCVR, and/or LCVR sequences set forth in Table 1 below.

The amount of antibody, or antigen-binding fragment thereof, contained within the pharmaceutical formulations of the present disclosure may vary depending on the specific properties desired of the formulations, as well as the particular circumstances and purposes for which the formulations are intended to be used. In certain aspects, the pharmaceutical formulations may contain about 1 mg/mL to about 500 mg/mL of antibody; about 5 mg/mL to about 250 mg/ml of antibody; about 5 mg/mL to about 200 mg/mL of antibody; about 15 mg/mL to about 200 mg/ml of antibody; about 25 mg/mL to about 200 mg/mL of antibody; about 50 mg/mL to about 200 mg/mL of antibody; about 100 mg/mL to about 200 mg/mL of antibody; about 125 mg/mL to about 175 mg/mL of antibody; or about 150 mg/mL to about 200 mg/mL of antibody. For example, the formulations of the present disclosure may be liquid formulations that comprise about 1 mg/ml; about 2 mg/mL; about 5 mg/mL; about 10 mg/mL; about 15 mg/mL; about 20 mg/mL; about 25 mg/mL; about 30 mg/mL; about 35 mg/mL; about 40 mg/mL; about 45 mg/mL; about 50 mg/mL; about 55 mg/mL; about 60 mg/mL; about 65 mg/mL; about 70 mg/mL; about 75 mg/mL; about 80 mg/mL; about 85 mg/mL; about 90 mg/mL; about 95 mg/mL; about 100 mg/mL; about 105 mg/mL; about 110 mg/mL; about 115 mg/mL; about 120 mg/mL; about 125 mg/mL; about 130 mg/mL; about 135 mg/mL; about 140 mg/mL; about 145 mg/mL; about 150 mg/mL; about 155 mg/mL; about 160 mg/mL; about 165 mg/mL; about 170 mg/mL; about 175 mg/mL; about 180 mg/mL; about 185 mg/mL; about 190 mg/mL; about 195 mg/mL; or about 200 mg/mL of an antibody or an antigen-binding fragment thereof that binds specifically to hIL-4Rα. In certain aspects, the pharmaceutical formulations are liquid formulations that may contain 5±0.5 mg/mL to 200±20 mg/mL of antibody; 15±1.5 mg/mL to 200±20 mg/mL of antibody; 25±2.5 mg/mL to 200±20 mg/mL of antibody; 50±5 mg/mL to 200±20 mg/mL of antibody; 100±10 mg/mL to 200±20 mg/mL of antibody; 150±10 mg/mL of antibody; or 175±10 mg/mL. In some aspects, the pharmaceutical formulations contain from 140±5 mg/mL to 160±5 mg/mL of the anti-IL-4R antibody. In some cases, the pharmaceutical formulations contain 165 mg/mL±5 mg/mL to 185 mg/mL±5 mg/mL of the anti-IL-4R antibody. In some cases, the pharmaceutical formulations contain 150 mg/mL±5 mg/mL of the anti-IL-4R antibody. In some cases, the pharmaceutical formulations contain 175 mg/mL±5 mg/mL of the anti-IL-4R antibody.

The present disclosure encompasses antibodies having amino acid sequences that vary from those of the exemplary molecules disclosed herein but that retain the ability to bind the cognate antigen, for example hIL-4R. Such variant molecules may comprise one or more additions, deletions, or substitutions of amino acids when compared to the parent sequence, but exhibit biological activity that is essentially equivalent to that of the antibodies discussed herein.

The present disclosure includes antigen-binding molecules that are bioequivalent to any of the exemplary antibodies set forth herein. In some aspects, the antigen-binding molecule is a bioequivalent of dupilumab. Two antibodies are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one aspect, two antibodies are bioequivalent if there are no clinically meaningful differences in their safety, purity, and potency. In one aspect, two antibodies are bioequivalent if a patient can be switched one or more times between the reference product and the biological product without an expected increase in the risk of adverse effects, including a clinically significant change in immunogenicity, or diminished effectiveness, as compared to continued therapy without such switching.

Bioequivalence may be demonstrated by in vivo and in vitro methods. Bioequivalence measures include, for example, (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) a well-controlled clinical trial that establishes safety, efficacy, or bioavailability or bioequivalence of an antigen-binding protein.

### Pharmaceutical Formulations

As used herein, a “sample” can be obtained from any step of a bioprocess, such as cell culture fluid (CCF), harvested cell culture fluid (HCCF), any step in the downstream processing, drug substance (DS), or a drug product (DP) comprising the final formulated product.

The terms “composition,” “formulation,” and “formulated drug substance” (FDS) as used in the present disclosure refer to a combination of two or more pharmaceutical ingredients for inclusion in a drug product. A composition, formulation, or FDS may be, for example, a liquid composition including an active pharmaceutical ingredient, such as an antibody, and an excipient, such as a stabilizer or surfactant. A composition, formulation, or FDS may include multiple excipients. A composition, formulation, or FDS may also include other constituents, such as host cell proteins co-purified with a protein of interest.

The term “drug product” (DP) as used in the present disclosure refers to a dosage form comprising an amount of a FDS for packaging, shipment, or administration. For example, a drug product may be a pre-filled syringe holding a volume of FDS for administration to a patient.

As used herein, a “protein pharmaceutical product,” “biopharmaceutical product,” “pharmaceutical formulation,” “pharmaceutical composition,” or “biotherapeutic” includes an active ingredient which can be fully or partially biological in nature. In one aspect, the protein pharmaceutical product can comprise a peptide, a protein, a fusion protein, an antibody, an antigen, vaccine, a peptide-drug conjugate, an antibody-drug conjugate, a protein-drug conjugate, cells, tissues, or combinations thereof. In another aspect, the protein pharmaceutical product can comprise a recombinant, engineered, modified, mutated, or truncated version of a peptide, a protein, a fusion protein, an antibody, an antigen, vaccine, a peptide-drug conjugate, an antibody-drug conjugate, a protein-drug conjugate, cells, tissues, or combinations thereof.

In some aspects, pharmaceutical formulations of the present invention comprise: (i) a human antibody that specifically binds to hIL-4Rα; (ii) one or more buffers; (iii) a thermal stabilizer; (iv) a surfactant (e.g., organic cosolvent); and (v) a viscosity modifier. Additional components may be included in the formulations of the present disclosure if such components do not significantly interfere with the viscosity and stability of the formulation. Specific exemplary components and formulations included within the present disclosure are described in detail below.

The pharmaceutical formulations of the present disclosure may, in certain aspects, be fluid formulations. As used herein, the expression “fluid formulation” means a mixture of at least two components that exists predominantly in the fluid state at about 2° C. to about 45° C. Fluid formulations include, inter alia, liquid formulations. Fluid formulations may be of low, moderate or high viscosity depending on their particular constituents.

### Host Cell Proteins

As used herein, the term “host cell protein” (HCP) includes protein derived from a host cell in the production of a recombinant protein. Host cell protein can be a process-related impurity which can be derived from the manufacturing process and can include three major categories: cell substrate-derived, cell culture-derived and downstream-derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables.

The presence of a host cell protein in a biotherapeutic product may be considered to be a higher or lower risk based on a number of measurable factors. One such factor is the concentration or abundance (quantity) of an HCP impurity in a biotherapeutic product. An HCP may have no discernible impact at a low enough abundance, as measured by, for example, ELISA or mass spectrometry. The level at which an HCP may present a considerable risk, which may be considered an unacceptable level in a product and may be monitored as a critical quality attribute (CQA), may depend on the specific identity of the HCP. Particular HCPs may be known to present a risk at a particular level, for example depending on the level of enzymatic activity of an HCP that is an enzyme.

Relatedly, the criticality of the presence of an HCP may depend on the function of that HCP, in particular in relation to the components of the biotherapeutic product. For example, an HCP esterase or lipase that may or is known to degrade polysorbate that is present in the biotherapeutic product of interest may be closely monitored and may have a low threshold for how much of the HCP impurity can be allowed in the biotherapeutic product. Other HCPs of particular concern may be, for example, proteases that may or are known to degrade a protein of interest in the biotherapeutic product, or immunogenic HCPs that may or are known to cause an immune reaction when administered to a subject.

### Liquid Chromatography

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, hydrophobic interaction chromatography, hydrophilic interaction chromatography, or mixed-mode chromatography. Analytes separated using chromatography will feature distinctive retention times, reflecting the speed at which an analyte moves through the chromatographic column. Analytes may be compared using a chromatogram, which plots retention time on one axis and measured signal on another axis, where the measured signal may be produced from, for example, UV detection or fluorescence detection. In some aspects, a sample including at least one esterase or lipase, for example a drug substance, may be subjected to stress, for example agitation stress or heat stress, and subsequently subjected to a chromatography step to remove any HMW species.

In certain aspects, it may be advantageous to subject a biological sample to affinity chromatography for production of a protein of interest. The chromatographic material is capable of selectively or specifically binding to or interacting with the protein of interest. Non-limiting examples of such chromatographic material include Protein A and Protein G. Also included is chromatographic material comprising, for example, a protein or portion thereof capable of binding to or interacting with the protein of interest.

Affinity chromatography can involve subjecting a biological sample to a column comprising a suitable Protein A resin. When used herein, the term “Protein A” encompasses Protein A recovered from a native source thereof, Protein A produced synthetically (e.g., by peptide synthesis or by recombinant techniques), and variants thereof which retain the ability to bind proteins which have a CH2/CH3 region. In certain aspects, Protein A resin is useful for affinity-based production and isolation of a variety of antibody isotypes by interacting specifically with the Fc portion of a molecule should it possess that region.

There are several commercial sources for Protein A resin. Suitable resins include, but are not limited to, MabSelect PrismA, MabSelect SuRe™, MabSelect SuRe LX, MabSelect, MabSelect SuRe pcc, MabSelect Xtra, rProtein A Sepharose from Cytiva, ProSep HC, ProSep Ultra, ProSep Ultra Plus from EMD Millipore, MabCapture from ThermoFisher, and Amsphere™ A3 from JSR Life Sciences.

An affinity column can be equilibrated with a suitable buffer prior to sample loading. Following loading of the column, the column can be washed one or multiple times using a suitable wash buffer. The column can then be eluted using an appropriate elution buffer, for example, glycine-HCl, acetic acid, or citric acid. The eluate can be monitored using techniques well known to those skilled in the art such as a UV detector. The eluted fractions of interest can be collected and then prepared for further processing.

Cation exchange chromatography (CEX) uses a cation exchange chromatography material. Cation exchange chromatography can be further subdivided into, for example, strong cation exchange (SCX) or weak cation exchange, depending on the cation exchange chromatography material employed. Cation exchange chromatography materials with a sulfonic acid group(S) may be used in strong cation exchangers, while cation exchange chromatography materials with a carboxymethyl group (CM) may be used in weak cation exchangers. Strong cation exchangers include, for example SOURCE S, which uses a functional group of methyl sulfate, and SP Sepharose, which uses a functional group of sulfopropyl. Weak cation exchangers include, for example, CM-Cellulose, which uses a functional group of carboxymethyl. SCX may be preferred because a wider range of pH buffers may be used without losing the charge of the strong cation exchanger, allowing for effective separation of analytes with a wide pI range.

Cation exchange chromatography materials are available under different names from a multitude of companies such as, for example, Bio-Rex, Macro-Prep CM (available from BioRad Laboratories, Hercules, Calif., USA), weak cation exchanger WCX 2 (available from Ciphergen, Fremont, Calif., USA), Dowex MAC-3 (available from Dow chemical company, Midland, Mich., USA), Mustang C (available from Pall Corporation, East Hills, N.Y., USA), Cellulose CM-23, CM-32, CM-52, hyper-D, and partisphere (available from Whatman plc, Brentford, UK), Amberlite IRC 76, IRC 747, IRC 748, GT 73 (available from Tosoh Bioscience GmbH, Stuttgart, Germany), CM 1500, CM 3000 (available from BioChrom Labs, Terre Haute, Ind., USA), and CM-Sepharose Fast Flow (available from GE Healthcare, Life Sciences, Germany). In addition, commercially available cation exchange resins further include carboxymethyl-cellulose, Bakerbond ABX, sulphopropyl (SP) immobilized on agarose (e.g. SP-Sepharose Fast Flow or SP-Sepharose High Performance, available from GE Healthcare-Amersham Biosciences Europe GmbH, Freiburg, Germany) and sulphonyl immobilized on agarose (e.g. S-Sepharose Fast Flow available from GE Healthcare, Life Sciences, Germany).

Cation exchange chromatography materials include mixed-mode chromatography materials performing a combination of ion exchange and hydrophobic interaction technologies (e.g., Capto adhere, Capto MMC, MEP HyperCell, Eshmuno HCX, etc.), mixed-mode chromatography materials performing a combination of anion exchange and cation exchange technologies (e.g., hydroxyapatite, ceramic hydroxyapatite, etc.), and the like. In some aspects, CEX may be used to remove HMW species from a sample, for example to remove proteins that have aggregated into HMW species following agitation or heat stress.

In some aspects, a sample comprising a protein of interest is subjected to at least one anion exchange (AEX) separation step. Anion exchange packed bed chromatography is based on ionic interactions between the binding entity (target protein or impurity) and the functional group immobilized on the chromatographic media. Performance may be a function of the mobile phase, the functional group, and the resin backbone. The use of an anionic exchange material versus a cationic exchange material is based, in part, on the local charges of the protein of interest. Anion exchange chromatography can be used in combination with other chromatographic procedures such as affinity chromatography, size exclusion chromatography, hydrophobic interaction chromatography as well as other modes of chromatography known to the skilled artisan.

In the context of chromatographic separation, a chromatographic column is used to house chromatographic support material (resin or solid phase). A sample comprising a protein of interest is loaded onto a particular chromatographic column. The column can then be subjected to one or more wash steps using a suitable wash buffer. Components of a sample that have not adsorbed onto the resin will likely flow through the column. Components that have adsorbed to the resin can be differentially eluted using an appropriate elution buffer.

An anionic agent may be selected from the group consisting of acetate, chloride, formate and combinations thereof. A cationic agent may be selected from the group consisting of Tris, arginine, sodium and combinations thereof. A buffer may be selected from the group consisting of pyridine, piperazine, L-histidine, Bis-Tris, Bis-Tris propane, imidazole, N-ethylmorpholine, TEA (triethanolamine), Tris, morpholine, N-methyldiethanolamine, AMPD (2-amino-2-methyl-1,3-propanediol), diethanolamine, ethanolamine, AMP (2-amino-2-methyl-1-propaol), piperazine, 1,3-diaminopropane and piperidine.

A packed anion-exchange chromatography column, anion-exchange membrane device, anion-exchange monolithic device, or depth filter media can be operated either in bind-elute mode, flowthrough mode, or a hybrid mode wherein proteins exhibit binding to the chromatographic material and yet can be washed from such material using a buffer that is the same or substantially similar to the loading buffer.

In the bind-elute mode, a column or membrane device is first conditioned with a buffer with appropriate ionic strength and pH under conditions where certain proteins will adsorb to the resin-based matrix. For example, during the feed load, a protein of interest can be adsorbed to the resin due to electrostatic attraction. After washing the column or the membrane device with the equilibration buffer or another buffer with a different pH and/or conductivity, the product recovery is achieved by increasing the ionic strength (i.e., conductivity) of the elution buffer to compete with the solute for the charged sites of the anion exchange matrix. Changing the pH and thereby altering the charge of the solute is another way to achieve elution of the solute. The change in conductivity or pH may be gradual (gradient elution) or stepwise (step elution).

In the flowthrough mode, a column or membrane device is operated at a selected pH and conductivity such that the protein of interest does not bind to the resin or the membrane while the acidic species will either be retained on the column or will have a distinct elution profile as compared to the protein of interest. In the context of this strategy, acidic species will interact with or bind to the chromatographic material under suitable conditions while the protein of interest and certain aggregates and/or fragments of the protein of interest will flow through the column.

In some aspects, an AEX step is performed in negative mode (flowthrough mode), where negatively charged process related impurities are adsorbed to the immobilize, positively charged ligand, and the protein of interest flows through.

Non-limiting examples of anionic exchange resins include diethylaminoethyl (DEAE), quaternary aminoethyl (QAE) and quaternary amine (Q) groups. Additional non-limiting examples include: Poros 50PI and Poros 50HQ, which are a rigid polymeric bead with a backbone consisting of cross-linked poly [styrene-divinylbenzene]; Poros 50XQ; Capto Q Impres and Capto DEAE, which are a high flow agarose bead; Capto Adhere; Q Sepharose Fast Flow; Toyopearl QAE-550, Toyopearl DEAE-650, and Toyopearl GigaCap Q-650, which are a polymeric base bead; Fractogel® EMD TMAE Hicap, which is a synthetic polymeric resin with a tentacle ion exchanger; Sartobind STICK PA nano, which is a salt-tolerant chromatographic membrane with a primary amine ligand; Sartobind Q nano, which is a strong anion exchange chromatographic membrane; CUNO BioCap, which is a zeta-plus depth filter media constructed from inorganic filter aids, refined cellulose, and an ion exchange resin; XOHC, which is a depth-filter media constructed from inorganic filter aid, cellulose, and mixed cellulose esters; and Unosphere Q. In some aspects, a resin is chosen with a relatively larger pore size, for increased surface area exposed to negatively charged species.

Additives such as polyethylene glycol (PEG), detergents, amino acids, sugars, chaotropic agents can be added to enhance the performance of the separation to achieve better separation, recovery and/or product quality.

In some aspects, AEX may be used to remove HMW species from a sample, for example to remove proteins that have aggregated into HMW species following agitation or heat stress.

Whereas ion exchange chromatography relies on the local charge of the protein of interest for selective separation, hydrophobic interaction chromatography (HIC) exploits the hydrophobic properties of proteins to achieve selective separation. HIC resins are typically functionalized with aromatic or aliphatic hydrocarbon ligands. Hydrophobic groups on or within a protein interact with hydrophobic groups of chromatography resin or a membrane. Typically, under suitable conditions, the more hydrophobic a protein is (or portions of a protein) the stronger it will interact with the column or the membrane. Thus, under suitable conditions, HIC can be used to facilitate the separation of process-related impurities (e.g., HCPs) as well as product-related substances (e.g., aggregates and fragments) from a protein of interest in a sample. In some aspects, HIC may be used to remove HMW species from a sample, for example to remove proteins that have aggregated into HMW species following agitation or heat stress.

Like ion exchange chromatography, a HIC column or a HIC membrane device can also be operated in an elution mode, a flowthrough, or a hybrid mode wherein the product exhibits binding to or interacting with a chromatographic material yet can be washed from such material using a buffer that is the same or substantially similar to the loading buffer. In some embodiments, a HIC step is performed in negative mode where process-related impurities bind to an immobilized ligand, and the protein of interest flows through.

As hydrophobic interactions are strongest at high ionic strength, this form of separation can be conveniently performed following a salt elution step such as those typically used in connection with ion exchange chromatography. Alternatively, salts can be added to a sample before employing a HIC step. Adsorption of a protein to a HIC column is favored by high salt concentrations, but the actual concentrations can vary over a wide range depending on the nature of the protein of interest, salt type and the particular HIC ligand chosen. Various ions can be arranged in a so-called soluphobic series depending on whether they promote hydrophobic interactions (salting-out effects) or disrupt the structure of water (chaotropic effect) and lead to the weakening of the hydrophobic interaction. Cations are ranked in terms of increasing salting out effect as Ba²⁺; Ca²⁺; Mg²⁺; Li⁺; Cs⁺; Na⁺; K⁺; Rb⁺; NH4⁺, while anions may be ranked in terms of increasing chaotropic effect as PO₄³⁻; SO₄²⁻; CH₃CO³⁻; CI⁻; Br; NO₃; ClO₄⁻; I⁻; SCN⁻.

HIC media normally comprise a base matrix (e.g., cross-linked agarose or synthetic copolymer material) to which hydrophobic ligands (e.g., alkyl or aryl groups) are coupled. A suitable HIC media comprises an agarose resin or a membrane functionalized with phenyl groups (e.g., a Phenyl Sepharose™ from Cytiva or a Phenyl Membrane from Sartorius). Various HIC resins are available commercially. Examples include, but are not limited to, Capto Phenyl, Capto Butyl, Phenyl Sepharose™ 6 Fast Flow with low or high substitution, Phenyl Sepharose™ High Performance, Octyl Sepharose™ High Performance (GE Healthcare); Fractogel™ EMD Propyl or Fractogel™ EMD Phenyl (E. Merck, Germany); Macro-Prep™ Methyl or Macro-Prep™ t-Butyl columns (Bio-Rad, California); WP HI-Propyl (C3)™ (J. T. Baker, New Jersey); and Toyopearl™ ether, phenyl or butyl (TosoHaas, PA); Toyo PPG; Toyo Phenyl; Toyo Butyl; and Toyo Hexyl.

Because the pH selected for any particular production process must be compatible with protein stability and activity, particular pH conditions may be specific for each application. However, because at pH 5.0-8.5 particular pH values have very little significance on the final selectivity and resolution of a HIC separation, such conditions may be favored. An increase in pH weakens hydrophobic interactions and retention of proteins changes more drastically at pH values above 8.5 or below 5.0. In addition, changes in ionic strength, the presence of organic solvents, temperature and pH (especially at the isoelectric point, pI, when there is no net surface charge) can impact protein structure and solubility and, consequently, the interaction with other hydrophobic surfaces, such as those in HIC media and hence, in certain embodiments, the present disclosure incorporates production strategies wherein one or more of the foregoing are adjusted to achieve the desired reduction in process-related impurities and/or product-related substances.

Size exclusion chromatography (SEC) or gel filtration relies on the separation of components as a function of their molecular size. Separation depends on the amount of time that the substances spend in the porous stationary phase as compared to time in the fluid. The probability that a molecule will reside in a pore depends on the size of the molecule and the pore. In addition, the ability of a substance to permeate into pores is determined by the diffusion mobility of macromolecules, which is higher for small macromolecules. Very large macromolecules may not penetrate the pores of the stationary phase at all; and for very small macromolecules, the probability of penetration is close to unity. While components of larger molecular size move more quickly past the stationary phase, components of small molecular size have a longer path length through the pores of the stationary phase and are thus retained longer in the stationary phase.

The chromatographic material can comprise a size exclusion material wherein the size exclusion material is a resin or membrane. The matrix used for size exclusion is preferably an inert gel medium which can be a composite of cross-linked polysaccharides, for example, cross-linked agarose and/or dextran in the form of spherical beads. The degree of cross-linking determines the size of pores that are present in the swollen gel beads. Molecules greater than a certain size do not enter the gel beads and thus move through the chromatographic bed the fastest. Smaller molecules, such as detergent, protein, DNA and the like, which enter the gel beads to varying extent depending on their size and shape, are retarded in their passage through the bed. Molecules are thus generally eluted in the order of decreasing molecular size.

Analytes eluting from an SEC column may be separated into fractions based on elution time. For example, analytes eluting earlier than the functional form of a protein of interest, for example the monomeric form, may be broadly categorized as high molecular weight (HMW) species. A HMW fraction may be further subdivided into, for example, a very high molecular weight (vHMW) fraction and a dimer fraction (representing the elution time of a dimer of the protein of interest). Analytes eluting later than the functional form of a protein of interest may be broadly categorized as low molecular weight (LMW) species, and may be further subdivided into a LMW fraction and a later tail fraction. Variants of a protein of interest that have a higher molecular weight or lower molecular weight than the main species, or than the intended product, may be referred to as “size variants.”

Similarly, sample components besides a protein of interest, for example a lipase, may form higher and lower molecular weight species that can be separated using SEC. HMW species may be depleted from a sample using a purification technique including, for example filtration, enrichment, or chromatography, for example size exclusion chromatography or cation exchange chromatography. In some aspects, SEC may be used to remove HMW species from a sample, for example to remove proteins that have aggregated into HMW species following agitation or heat stress. As used herein, the term “HMW-depleted” refers to a sample that has been subjected to a purification or enrichment step to substantially (but not necessarily completely) remove, or lower the relative abundance of, any HMW species, compared to the sample prior to the purification or enrichment step, and/or compared to a sample that was not subjected to the purification or enrichment step. A HMW-depleted sample may comprise, for example, HMW species as a percentage of total HMW, main, and LMW species that is less than 10%, less than 5%, less than 4%, less than 3%, less than 2%, or less than 1%. For pharmaceutical compositions, a particular threshold used in the field for HMW depletion is a percent of HMW species less than 5%.

Distinguishing a HMW species from a main species is easily accomplished by a person skilled in the art, for example by the visible distinction in SEC peaks between the different species.

### Mass Spectrometry

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

In some aspects, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. MS/MS, or MS², can be performed by first selecting and isolating a precursor ion (MS¹), and fragmenting it to obtain meaningful information. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m z separation functions can be designed so that in one section of the instrument ions are selected, dissociated in an intermediate region, and the product ions are then transmitted to another analyzer for m z separation and data acquisition. In tandem-in-time, mass spectrometer ions produced in the ion source can be trapped, isolated, fragmented, and m z separated in the same physical device.

The peptides identified by the mass spectrometer can be used as surrogate representatives of the intact protein and their post-translational modifications or other modifications. They can be used for protein characterization by correlating experimental and theoretical MS/MS data, the latter generated from possible peptides in a protein sequence database. The characterization can include, but is not limited to, sequencing amino acids of the protein fragments, determining protein sequencing, determining protein de novo sequencing, locating post-translational modifications, or identifying post-translational modifications, or comparability analysis, or combinations thereof.

In some exemplary aspects, the mass spectrometer can work on nanoelectrospray or nanospray. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

In some aspects, mass spectrometry can be performed under native conditions. As used herein, the term “native conditions” can include performing mass spectrometry under conditions that preserve non-covalent interactions in an analyte. For a detailed review on native MS, refer to the review: Elisabetta Boeri Erba & Carlo Petosa, The emerging role of native mass spectrometry in characterizing the structure and dynamics of macromolecular complexes, 24 PROTEIN SCIENCE 1176-1192 (2015).

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools.” Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com/proteinpilot), Phenyx (www.phenyx-ms.com), Sorcerer (www.sagenresearch.com), OMSSA (www.pubchem.ncbi.nlm.nih.gov/omssa/), X!Tandem (www.thegpm.org/TANDEM/), Protein Prospector (prospector.ucsf.edu/prospector/mshome.htm), Byonic (www.proteinmetrics.com/products/byonic) or Sequest (fields.scripps.edu/sequest).

### Formulation Excipients

The pharmaceutical formulations of the present disclosure comprise one or more excipients. The term “excipient,” as used herein, means any non-therapeutic agent added to the formulation to provide a desired consistency, viscosity or stabilizing effect.

The pharmaceutical formulations of the present disclosure may also comprise a buffer or buffer system, which serves to maintain a stable pH and to help stabilize the protein of interest. In some aspects, the buffer or buffer system comprises at least one buffer that has a buffering range that overlaps fully or in part the range of pH 5.5 to 6.3. In various aspects, the pH of the formulation is 5.5, 5.6, 5.7, 5.8, 5.9, 6.0, 6.1, 6.2 or 6.3. In some aspects, the formulations have a pH of 5.9±0.3. In some aspects, the formulations have a pH of 5.9±0.2. In some aspects, the formulations have a pH of 5.9±0.1. In certain aspects, the buffer comprises a histidine buffer. In certain aspects, the buffer comprises an acetate buffer. In certain aspects, the buffer (e.g., histidine and/or acetate) is present at a concentration of from about 1 mM to about 40 mM, about 5 mM to about 30 mM, about 10 mM to about 15 mM; or about 15 mM to about 25 mM. In some aspects, the buffer includes a histidine buffer at a concentration of from 15 mM to 25 mM. In some aspects, the buffer includes a histidine buffer at a concentration of 20 mM±2 mM. In some cases, the histidine buffer is present at a concentration of 15 mM, 16 mM, 17 mM, 18 mM, 19 mM, 20 mM, 21 mM, 22 mM, 23 mM, 24 mM, or 25 mM. In some aspects, the buffer comprises an acetate buffer at a concentration of from 10 mM to 15 mM. In some aspects, the buffer comprises an acetate buffer at a concentation of 12.5 mM±1.25 mM. In some cases, the acetate buffer is present at a concentration of 10 mM, 10.5 mM, 11 mM, 11.5 mM, 12 mM, 12.5 mM, 13 mM, 13.5 mM, 14 mM, 14.5 mM, or 15 mM. In some aspects, the formulations of the present disclosure include both histidine and acetate buffers at any of the concentrations discussed above. In some cases, the formulations contain a histidine buffer at a concentration of from 15 mM to 25 mM, and an acetate buffer at a concentration of from 10 mM to 15 mM. In some cases, the formulations contain a histidine buffer at a concentration of 20 mM±2 mM, and an acetate buffer at a concentration of 12.5 mM=1.25 mM.

The pharmaceutical formulations of the present disclosure may also comprise one or more carbohydrates, e.g., one or more sugars. The sugar can be a reducing sugar or a non-reducing sugar. “Reducing sugars” include, e.g., sugars with a ketone or aldehyde group and contain a reactive hemiacetal group, which allows the sugar to act as a reducing agent. Specific examples of reducing sugars include fructose, glucose, glyceraldehyde, lactose, arabinose, mannose, xylose, ribose, rhamnose, galactose and maltose. Non-reducing sugars can comprise an anomeric carbon that is an acetal and is not substantially reactive with amino acids or polypeptides to initiate a Maillard reaction. Specific examples of non-reducing sugars include sucrose, trehalose, sorbose, sucralose, melezitose and raffinose. Sugar acids include, for example, saccharic acids, gluconate and other polyhydroxy sugars and salts thereof. In some aspects, the sugar is sucrose. In some cases, the sugar (e.g., sucrose) acts as a thermal stabilizer for the protein of interest.

The amount of sugar (e.g., sucrose) contained within the pharmaceutical formulations of the present disclosure will vary depending on the specific circumstances and intended purposes for which the formulations are used. In certain aspects, the formulations may contain about 0.1% to about 20% sugar; about 0.5% to about 20% sugar; about 1% to about 20% sugar; about 2% to about 15% sugar; about 3% to about 8% sugar; or about 4% to about 6% sugar. For example, the pharmaceutical formulations of the present disclosure may comprise about 0.5%; about 1.0%; about 1.5%; about 2.0%; about 2.5%; about 3.0%; about 3.5%; about 4.0%; about 4.5%; about 5.0%; about 5.5%; about 6.0%; about 6.5%; about 7.0%; about 7.5%; about 8.0%; about 8.5%; about 9.0%; about 9.5%; about 10.0%; about 15%; or about 20% sugar (e.g., sucrose). In some aspects, the formulations contain about 5% sugar (e.g., sucrose). In some aspects, the formulations contain about 5%±0.5% sugar (e.g., sucrose). Each of the percentages noted above corresponds to a percent weight/volume (w/v).

In certain aspects, the pharmaceutical formulations of the disclosure comprise at least one amino acid. In some aspects, the amino acid is arginine. In some aspects, the arginine is provided in the form of arginine hydrochloride. In some cases, the amino acid (e.g., arginine) acts as a viscosity modifier for the formulations of the protein of interest.

The amount of amino acid contained within the pharmaceutical formulations of the present disclosure may vary depending on the specific properties desired of the formulations, as well as the particular circumstances and purposes for which the formulations are intended to be used. In certain aspects, the formulations may contain about 1 mM to about 200 mM of an amino acid; about 5 mM to about 150 mM of an amino acid; about 10 mM to about 100 mM of an amino acid; about 20 mM to about 80 mM of an amino acid; about 20 mM to about 30 mM of an amino acid; about 45 mM to about 55 mM of an amino acid; or about 70 mM to about 80 mM of an amino acid. For example, the pharmaceutical formulations of the present disclosure may comprise about 5 mM; about 10 mM; about 15 mM; about 20 mM; about 25 mM; about 30 mM; about 35 mM; about 40 mM; about 45 mM; about 50 mM; about 55 mM; about 60 mM; about 65 mM; about 70 mM; about 75 mM; about 80 mM; about 85 mM; about 90 mM; about 95 mM; or about 100 mM of an amino acid (e.g., arginine). In some aspects, the formulations contain about 25 mM of an amino acid (e.g., arginine). In some aspects, the formulations contain about 50 mM of an amino acid (e.g., arginine). In some aspects, the formulations contain about 75 mM of an amino acid (e.g., arginine).

The pharmaceutical formulations of the present disclosure may also comprise one or more organic cosolvents in a type and in an amount that stabilizes the protein of interest under conditions of rough handling or agitation, such as, e.g., orbital shaking. In some aspects, the organic cosolvent is a surfactant. As used herein, the term “surfactant” means a substance which reduces the surface tension of a fluid in which it is dissolved and/or reduces the interfacial tension between oil and water. Surfactants can be ionic or non-ionic. Specific non-ionic surfactants that can be included in the formulations of the present disclosure include, for examples, polysorbates such as PS20 and PS80, poloxamers such as poloxamer 188, and polyethylene glycols (PEGs) such as PEG3350.

The amount of surfactant contained within the pharmaceutical formulations of the present disclosure may vary depending on the specific properties desired of the formulations, as well as the particular circumstances and purposes for which the formulations are intended to be used. In certain aspects, the formulations may contain at least about 0.01% surfactant. In certain aspects, the formulations may contain less than 0.2% surfactant. In some aspects, the formulations may contain less than 0.5% surfactant. In certain aspects, the formulations may contain about 0.01% to about 0.49% surfactant; about 0.01% to about 0.39% surfactant; about 0.01% to about 0.29% surfactant; about 0.01% to about 0.19% surfactant; about 0.01% to about 0.15% surfactant; about 0.01% to about 0.12%; about 0.01% to about 0.11% surfactant; about 0.01% to about 0.1% surfactant; or about 0.01% to about 0.09% surfactant. For example, the formulations of the present disclosure may comprise about 0.01%; about 0.02%; about 0.03%; about 0.04%; about 0.05%; about 0.06%; about 0.07%; about 0.08%; about 0.09%; about 0.1%; about 0.11%; about 0.12%; about 0.13%; about 0.14%; about 0.15%; about 0.16%; about 0.17%; about 0.18%; about 0.19%; about 0.20%; about 0.21%; about 0.22%; about 0.23%; about 0.24%; about 0.25%; about 0.26%; about 0.27%; about 0.28%; about 0.29%; about 0.30%; about 0.35%; about 0.40%; about 0.45%; or about 0.50% surfactant (e.g., PS20, PS80, poloxamer 188 or PEG3350). Each of the percentages noted above corresponds to a percent weight/volume (w/v).

### Polysorbates

In some aspects, the surfactant in the composition can be a polysorbate. As used herein, “polysorbate” refers to a common excipient used in formulation development to protect antibodies against various physical stresses such as agitation, freeze-thaw processes, and air/water interfaces (Emily Ha, Wei Wang & Y. John Wang, Peroxide formation in polysorbate 80 and protein stability, 91 JOURNAL OF PHARMACEUTICAL SCIENCES 2252-2264 (2002); Bruce A. Kerwin, Polysorbates 20 and 80 Used in the Formulation of Protein Biotherapeutics: Structure and Degradation Pathways, 97 JOURNAL OF PHARMACEUTICAL SCIENCES 2924-2935 (2008); Hanns-Christian Mahler et al., Adsorption Behavior of a Surfactant and a Monoclonal Antibody to Sterilizing-Grade Filters, 99 Journal of Pharmaceutical Sciences 2620-2627 (2010)) and can include a non-ionic, amphipathic surfactant composed of fatty acid esters of polyoxyethylene-sorbitan. The esters can include polyoxyethylene sorbitan head group and either a saturated monolaurate side chain (polysorbate 20; PS20) or an unsaturated monooleate side chain (polysorbate 80; PS80). In some aspects, the polysorbate can be present in the formulation in the range of about 0.001% to 1% (weight/volume). Polysorbate can also contain a mixture of various fatty acid chains; for example, polysorbate 80 contains oleic, palmitic, myristic and stearic fatty acids, with the monooleate fraction making up approximately 58% of the polydisperse mixture (Nitin Dixit et al., Residual Host Cell Protein Promotes Polysorbate 20 Degradation in a Sulfatase Drug Product Leading to Free Fatty Acid Particles, 105 JOURNAL OF PHARMACEUTICAL SCIENCES 1657-1666 (2016)). Non-limiting examples of polysorbates include polysorbate-20, polysorbate-40, polysorbate-60, polysorbate-65, and polysorbate-80.

A polysorbate can be susceptible to auto-oxidation in a pH- and temperature-dependent manner, and additionally, exposure to UV light can also produce instability (Ravuri S. k. Kishore et al., Degradation of Polysorbates 20 and 80: Studies on Thermal Autoxidation and Hydrolysis, 100 JOURNAL OF PHARMACEUTICAL SCIENCES 721-731 (2011)), resulting in free fatty acids in solution along with the sorbitan head group. The free fatty acids resulting from polysorbate can include any aliphatic fatty acids with six to twenty carbons. Non-limiting examples of free fatty acids include oleic acid, palmitic acid, stearic acid, myristic acid, lauric acid, or combinations thereof.

In some exemplary aspects, the polysorbate can form free fatty acid particles. The free fatty acid particles can be at least about 1 μm in size or at least about 5 μm in size. Further, these fatty acid particles can be classified according to their size as visible (about >100 μm), sub-visible (about≤100 μm, which can be sub-divided into micron (1-100 μm) and submicron (100 nm-1000 nm)) and nanometer particles (about≤100 nm) (Linda Narhi, Jeremy Schmit & Deepak Sharma, Classification of protein aggregates, 101 JOURNAL OF PHARMACEUTICAL SCIENCES 493-498). In some exemplary aspects, the fatty acid particles can be visible particles. Visible particles can be determined by visual inspection. In some aspects, the fatty acid particles can be sub-visible particles. Subvisible particles can be monitored by the light blockage method according to United States Pharmacopeia (USP). An increase in fatty acid particles may cause a product to no longer be of acceptable quality, and therefore a rate of increase of fatty acid particles may be used as a measure of product shelf life. Fatty acid particles may form when free fatty acids are released into a formulation and exceed a concentration at which they are soluble, thereby precipitating out of solution. Therefore, measuring a degradation of polysorbate or a concentration of released free fatty acids may be indicators of the formation of fatty acid particles, and by extension of predicted product shelf life. Additionally, preventing a degradation of polysorbate, a release of free fatty acids, and/or a formation of fatty acid particles may be important for extending product shelf life and improving product quality.

In some exemplary aspects, the concentration of polysorbate in the formulation can be about 0.001% w/v, about 0.002% w/v, about 0.003% w/v, about 0.004% w/v, about 0.005% w/v, about 0.006% w/v, about 0.007% w/v, about 0.008% w/v, about 0.009% w/v, about 0.01% w/v, about 0.015% w/v, about 0.02% w/v, 0.025% w/v, about 0.03% w/v, about 0.035% w/v, about 0.04% w/v, about 0.045% w/v, about 0.05% w/v, about 0.06% w/v, about 0.07% w/v, about 0.08% w/v, about 0.09% w/v, about 0.1% w/v, about 0.2% w/v, about 0.3% w/v, about 0.4% w/v, about 0.5% w/v, about 0.6% w/v, about 0.7% w/v, about 0.8% w/v, about 0.9% w/v, or about 1% w/v. In one aspect, the concentration of polysorbate in the formulation is about 1% w/v.

In some exemplary aspects, the concentration of free fatty acids in the formulation can be about 10 ng/ml, about 20 ng/mL, about 30 ng/ml, about 40 ng/ml, about 50 ng/ml, about 60 ng/mL, about 70 ng/mL, about 80 ng/mL, about 90 ng/mL, about 100 ng/ml, about 200 ng/ml, about 300 ng/mL, about 400 ng/ml, about 500 ng/ml, about 600 ng/ml, about 700 ng/ml, about 800 ng/mL, about 900 ng/mL, about 1 μg/mL, about 2 μg/mL, about 3 μg/mL, about 4 μg/mL, about 5 g/mL, about 6 μg/mL, about 7 μg/mL, about 8 μg/mL, about 9 μg/mL, about 10 μg/mL, about 20 μg/mL, about 30 μg/mL, or about 40 μg/mL.

In some exemplary aspects, the polysorbate can be degraded by a host cell protein present in the composition. In some aspects, the host cell protein may be an esterase or lipase. Residual esterase or lipase activity in a formulation may be indirectly assessed by measuring polysorbate degradation, release of free fatty acids, or a concentration of visible or subvisible fatty acid particles.

The term “fatty acid ester” means any organic compound that contains a fatty acid chain linked to a head group via an ester bond. An ester bond is formed when a hydroxyl group (e.g., an alcohol or carboxylic acid) is replaced by an alkoxy group. As used herein, the hydroxyl group can be part of a carboxylic acid, more specifically a fatty acid, and/or an alcohol, such as glycerol, sorbitol, sorbitan, isosorbide, or the like. The alcohol group is generally referred to herein as the head group.

Examples of fatty acid esters generally include phospholipids, lipids (e.g., the head group is glycerol, including monoglycerides, diglycerides, and triglycerides), and surfactants and emulsifiers, including for example polysorbates like polysorbate 20, polysorbate 60, and polysorbate 80, which are non-ionic detergents. Surfactants and emulsifiers are useful as cosolvents and stabilizers. They function by associating with both a hydrophilic surface and a lipophilic surface to maintain dispersion and structural stability of ingredients, like proteins. Surfactants are added to protein formulations primarily to enhance protein stability against mechanical stress, such as air/liquid interface and solid/liquid interface shear. Without a surfactant, proteins may in some cases become structurally unstable in solution, and form multimeric aggregates that eventually become subvisible particles.

The term “fatty acid” or “fatty acid chain” means a carboxylic acid having an aliphatic tail. An aliphatic tail is simply a hydrocarbon chain comprising carbon and hydrogen, and in some cases, oxygen, sulfur, nitrogen and/or chlorine substitutions. Aliphatic tails can be saturated (as in saturated fatty acids), which means that all carbon-carbon bonds are single bonds (i.e., alkanes). Aliphatic tails can be unsaturated (as in unsaturated fatty acids), wherein one or more carbon-carbon bonds are double bonds (alkenes), or triple bonds (alkynes).

Fatty acids are generally designated as short-chain fatty acids, which have fewer than six carbons in their aliphatic tails, medium-chain fatty acids having six to twelve carbons, long-chain fatty acids having thirteen to twenty one carbons, and very long chain fatty acids having aliphatic tails of twenty two carbons and longer. As mentioned above, fatty acids are also categorized according to their degree of saturation, which correlates to stiffness and melting point. Common fatty acids include caprylic acid (8 carbons: 0 double bonds; 8:0), capric acid (10:0), lauric acid (12:0), myristic acid (14:0), myristoleic acid (14:1), palmitic acid (16:0), palmitoleic acid (16:1), sapienic acid (16:1), stearic acid (18:0), oleic acid (18:1), elaidic acid (18:1), vaccenic acid (18:1), linoleic acid (18:2), linelaedic acid (18:2), alpha-linolenic acid (18:3), arachidic acid (20:0), arachidonic acid (20:4), eicosapentaenoic acid (20:5), behenic acid (22:0), erucic acid (22:1), docosahexaenoic acid (22:6), lignoceric acid (24:0), and cerotic acid (26:0).

As mentioned above, polysorbates are fatty acid esters useful as non-ionic surfactants and protein stabilizers. Polysorbate 20, polysorbate 40, polysorbate 60, and polysorbate 80 are widely employed in the pharmaceutical, cosmetic, and food industries as stabilizers and emulsifiers. Polysorbate 20 mostly comprises the monolaurate ester of polyoxyethylene (20) sorbitan. Polysorbate 40 mostly comprises the monopalmitate ester of polyoxyethylene (20) sorbitan. Polysorbate 60 mostly comprises the monostearate ester of polyoxyethylene (20) sorbitan. Polysorbate 80 mostly comprises the monooleate ester of polyoxyethylene (20) sorbitan.

The quality of commercial grades of polysorbates varies from vendor to vendor. Polysorbates therefore are often mixtures of various chemical entities, consisting mostly of polyoxyethylene (20) sorbitan monoesters (as described above) with, in some cases, isosorbide ester contaminants. The head group (in this case polyoxyethylene (20) sorbitan) comprises a sorbitan (a mixture of dehydrated sorbitols, including 1,4-anhydrosorbitol, 1,5-anhydrosorbitol, and 1,4,3,6-dianhydrosorbitol) substituted at three of its alcohol groups to form ether bonds with three polyoxyethylene groups. The fourth alcohol group is substituted with a fatty acid to form a fatty acid ester.

In some commercially available batches of polysorbates, the polysorbate contains isosorbide monoesters. Isosorbide is a heterocyclic derivative of glucose, also prepared by the dehydration of sorbitol. It is a diol having two alcohol groups that can take part in the formation of one or two ester bonds. Thus, for example, some lots of polysorbate 20 can contain significant amounts of isosorbide laurate mono- and di-esters, and some lots of polysorbate 80 can contain significant amounts of isosorbide oleate mono- and di-esters.

In addition to head group variation, preparations of polysorbates contain variable amounts of other fatty acid esters. For example, an analysis of one particular source of polysorbate 20 revealed <10% caprylic acid, <10% capric acid, 40-60% lauric acid, 14-25% myristic acid, 7-15% palmitic acid, <11% oleic acid, <7% stearic acid, and <3% linoleic acid. An analysis of a polysorbate 80 batch revealed <5% myristic acid, <16% palmitic acid, >58% oleic acid, <6% stearic acid, and <18% linoleic acid. An analysis of another source of polysorbate 80 revealed about 70% oleic acid, with the remainder being other fatty acid esters and impurities. An analysis of yet another source of polysorbate 80 revealed about 86-87% oleic acid. An analysis of a further, more recently-developed source of polysorbate 80 revealed ≥99% oleic acid.

In some aspects, a concentration of oleic acid in polysorbate 80 may be between about 50% and about 100%, between about 58% and about 100%, between about 60% and 100%, between about 80% and about 100%, between about 90% and about 100%, between about 95% and about 100%, between about 98% and about 100%, between about 99% and about 100%, between about 98.0% and about 99.9%, between about 98.5% and about 99.5%, between about 99.0% and about 99.9%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.5%, at least 99%, at least 99.5%, about 58%, about 60%, about 65%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 98.5%, about 99%, about 99.1%, about 99.2%, about 99.3%, about 99.4%, about 99.5%, about 99.6%, about 99.7%, about 99.8%, about 99.9%, or about 100%.

Biopharmaceutical drugs are often formulated with non-ionic detergents like polysorbate 20 or polysorbate 80. These detergents help stabilize large molecules like antibodies and other proteins, and help prevent the formation of supermolecular ternary complexes or other aggregates. Aggregates can become nanoparticles or subvisible particles in the 10 to 100 micron range or 2 to 100 micron range, and interfere with drug product stability and shelf-life. Therefore, the stability of protein formulations depends in some cases upon the stability of the non-ionic detergent additive. However, and as is further discussed herein, polysorbate 20 and polysorbate 80 can, in some instances, contribute to the formation of aggregates, nanoparticles, and subvisible particles.

The phrase “subvisible particle” means a particle that is not visible, especially in a liquid. In other words, a solution or other liquid containing subvisible particles, but not visible particles, will not appear cloudy. Subvisible particles generally include those particles 100 microns or less in diameter, but in some cases include particles under 150 microns (Narhi et al., “A critical review of analytical methods for subvisible and visible particles,” Curr Pharm Biotechnol 10 (4): 373-381 (2009)). Subvisible particles may be the result of foreign contaminants or protein aggregation. Protein aggregates can be soft and amorphous in shape and therefore may be difficult to detect using conventional light obscuration and microscopic methods (Singh and Toler, “Monitoring of subvisible particles in therapeutic proteins,” Methods Mol Biol. 2012; 899:379-401). Subvisible particles may comprise, inter alia, silicone contaminants (oily droplets), free fatty acids (oily droplets), aggregated protein (amorphous particles), and/or protein/fatty acid complexes (amorphous particles).

Subvisible particles can be detected by any one or more of various methods. The USP standards specify light obscuration and optical microscopy protocols. Other methods include micro-flow image (MFI) analysis, Coulter counting, and submicron particle tracking methods. For light obscuration (LO), particles are counted based on the shadows they cast upon a light detector as they pass through a light beam in a flow cell. The size, shape and inverse intensity of the shadow depends upon the size, shape and difference in the refractive index of the particle relative to the solution. The lower size range for detection using LO is about 2 microns. A commonly used LO device is the HIAC instrument (Beckman Coulter, Brea, Calif.). Several methods of measurement and characterization of SVPs (e.g., light obscuration, flow microscopy, the electrical sensing zone method, and flow cytometry), are discussed in, for example, Narhi et al., “Subvisible (2-100 μm) Particle Analysis During Biotherapeutic Drug Product Development: Part 1, Considerations and Strategy,” J. Pharma. Sci. 104:1899-1908 (2015).

Light obscuration is criticized for underestimating protein aggregates and other amorphous structures. Flow image analysis, such as micro-flow imaging (MFI) (Brightwell Technologies, Ottawa, Ontario), is a more sensitive method of detecting the irregularly shaped, fragile, and transparent proteinaceous subvisible particles, and of differentiating those types of particles from silicone micro-droplets, air bubbles, and other foreign contaminants (Sharma et al., “Micro-flow imaging: Flow microscopy applied to sub-visible particulate analysis in protein formulations,” AAPS J. 12 (3): 455-464 (2010)). In general, because SVP measurement and characterization by light obscuration analysis is less sensitive than MFI, particle counts detected by MFI will tend to be higher than particle counts detected by light obscuration analysis. Briefly, MFI is flow microscopy in which successive bright field images are taken and analyzed in real time. Image analysis algorithms are applied to the images to discriminate air bubbles, silicone oil droplets, and proteinaceous aggregates. Volumes as low as about 250 microliters to as high as tens of milliliters can be analyzed. Depending on the system used, particles in the range of two to 300 microns, or one to 70 microns can be detected (Id).

In some aspects, visible or subvisible particles in a formulation can be detected and analyzed by Raman spectroscopy. As used herein, the term “Raman spectroscopy” refers to a spectroscopic method based on Raman scattering method. Raman spectroscopy can provide a Raman spectrum, which can identify the presence and position of bands in the fingerprint region (2000 to 400 cm-1) which enables the chemical identification of the analyzed material by comparison with a database of Raman spectra (C. V. Raman and K. S. Krishnan, A new type of secondary radiation, 121 NATURE 501-502 (1928); Zai-Qing Wen, Raman spectroscopy of protein pharmaceuticals, 96 JOURNAL OF PHARMACEUTICAL SCIENCES 2861-287 (2007)).

The FDA and other government regulatory agencies have placed limits on the amount of subvisible particles allowed in parenteral drug formulations. The major articulated concern is the uncertainty surrounding potential immunogenicity and downstream negative effects in the patient receiving the drug (Singh et al., “An industry perspective on the monitoring of subvisible particles as a quality attribute for protein therapeutics,” J. Pharma. Sci. 99 (8): 3302-21 (2010)). For small volume parenteral drugs (e.g., 100 mL or below), the pharmacopeia limits subvisible particles (SVP) of greater than or equal to 10 microns to no more than 6,000 SVPs per container, and SVPs of greater than or equal to 25 microns to no more than 600 per container, when determined by light obscuration analysis; and SVPs of greater than or equal to 10 microns to no more than 3,000 SVPs per container, and SVPs of greater than or equal to 25 microns to no more than 300 per container, when determined by the membrane microscopic test. (United States Pharmacopeia and National Formulary (USP 40-NF 28), <787> Subvisible Particulate Matter in Therapeutic Protein Injections.) For ophthalmic drugs, the SVP limits are 50 per mL of 10 microns or greater, 5 per mL of 25 microns or greater, and 2 per mL of 50 microns or greater (Id. at <78922 Particulate Matter in Ophthalmic Solutions). Regulatory agencies are increasingly contemplating that manufacturers establish specifications for SVPs of 2 microns or greater (see Singh et al., “An industry perspective on the monitoring of subvisible particles as a quality attribute for protein therapeutics,” J. Pharm. Sci. 99 (8): 3302-21 (2010)).

The term “esterase” means an enzyme that catalyzes the hydrolysis of an ester bond to create an acid and an alcohol. Esterases are a diverse category of enzymes, including acetyl esterases (e.g., acetylcholinesterase), phosphatases, nucleases, thiolesterases, lipases and other carboxyl ester hydrolases. As its name implies, a carboxyl ester hydrolase (a.k.a. carboxylesterase, carboxylic-ester hydrolase, and EC 3.1.1.1) uses water to hydrolyze a carboxylic ester into an alcohol and a carboxylate. A lipase is a carboxyl ester hydrolase that catalyzes the hydrolysis of lipids, including triglycerides, fats and oils into fatty acids and an alcohol head group. For example, triglycerides are hydrolyzed by lipases like pancreatic lipase to form monoacylglycerol and two fatty acid chains.

Phospholipases are lipases that hydrolyze phospholipids into fatty acids and other products. Phospholipases fall into four broad categories: phospholipase A (including phospholipase A1 and phospholipase A2), phospholipase B, and the phosphodiesterases phosphodiesterase C and phosphodiesterase D. In addition to the canonical phospholipases, phospholipase B-like enzymes, which reside at the lysosome lumen, are thought to be involved in lipid catalysis. For example, phospholipase B-like 2 (PLBL2) is postulated to have esterase activity based upon sequence homology and subcellular localization (Jensen et al., “Biochemical characterization and liposomal localization localization of the mannose-6-phosphate protein p76,” Biochem. J. 402:449-458 (2007)).

As used herein, the phrase “percent fatty ester hydrolysis” means the molar proportion of fatty acid ester that has been hydrolyzed. Since hydrolysis of a fatty acid ester results in the release of a free fatty acid, the percent fatty ester hydrolysis can be determined by measuring the free fatty acid in a sample. Therefore, percent fatty ester hydrolysis may be determined by calculating moles of free fatty acid divided by the sum of moles of fatty acid plus moles of fatty acid ester. In the case of percent hydrolysis of polysorbate 80 or polysorbate 20, that number may be determined by calculating the moles of free fatty acid and dividing by the total moles of remaining polysorbate plus moles of free fatty acid.

The term “esterase inhibitor” means any chemical entity that reduces, inhibits, or blocks the activity of an esterase. The applicants envision that the inclusion of an esterase inhibitor in a protein formulation containing a fatty acid ester surfactant may help maintain protein stability and fatty acid ester stability and help reduce SVP formation. Common esterases known in the art include orlistat (tetrahydrolipistatin; an inhibitor of carboxylesterase 2 and lipoprotein lipase), diethylumbelliferyl phosphate (a cholesterol esterase [lipsase A] inhibitor), URB602 ([1-1′-biphenyl]-3-tl-carbamicacid cyclohexyl ester; a monoacylglycerol lipase inhibitor), and 2-butoxyphenylboronic acid (an inhibitor of hormone-sensitive lipase). The inclusion of an esterase inhibitor during purification of a protein of interest or in the final formulation is expected to prevent or slow the hydrolysis of non-ionic detergents like polysorbate 20 and polysorbate 80, which in turn is expected to prevent or reduce subvisible particle formation.

In any of the various aspects of the pharmaceutical formulations discussed above or herein, the human IL-4R antibody may comprise a human IgG1 heavy chain constant region.

In any of the various aspects of the pharmaceutical formulations discussed above or herein, the human IL-4R antibody may comprise a human IgG4 heavy chain constant region.

In some aspects, the human IL-4R antibody may comprise a heavy chain comprising the amino acid sequence of SEQ ID NO: 9 and a light chain comprising the amino acid sequence of SEQ ID NO: 10.

Additional non-limiting examples of pharmaceutical formulations encompassed by the present disclosure are set forth elsewhere herein, including the working Examples presented below.

### Stability of the Pharmaceutical Formulations

The pharmaceutical formulations of the present disclosure exhibit high levels of stability. The term “stable,” as used herein in reference to the pharmaceutical formulations, means that the proteins of interest within the pharmaceutical formulations retain an acceptable degree of structure and/or function and/or biological activity after storage for a defined amount of time. A formulation may be stable even though the protein contained therein does not maintain 100% of its structure and/or function and/or biological activity after storage for a defined amount of time. Under certain circumstances, maintenance of about 90%, about 95%, about 96%, about 97%, about 98% or about 99% of a protein's structure and/or function and/or biological activity after storage for a defined amount of time may be regarded as “stable.”

Stability can be measured, inter alia, by determining the percentage of protein that forms an aggregate within the formulation after storage for a defined amount of time at a defined temperature, or under stress conditions (e.g., agitation), wherein stability is inversely proportional to the percent aggregate that is formed. The percentage of aggregated protein can be determined by, inter alia, size exclusion chromatography (e.g., size exclusion high performance liquid chromatography (SE-HPLC) or size exclusion ultra-performance liquid chromatography (SE-UPLC)). An “acceptable degree of stability”, as that phrase is used herein, means that at most about 15%, 10%, 5%, 4%, 3%, 2.5%, 2%, 1.5%, 1%, 0.5%, or 0.1% of the protein can be detected in an aggregate in the formulation after storage for a defined amount of time at a given temperature, or under specified stress conditions. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 4° C.-8° C., about 5° C., about 25° C., about 35° C., about 37° C. or about 45° C. The “stress condition” to which the formulated protein of interest may be subjected may be agitation stress (e.g., vortexing) for a period of 10 minutes, 20 minutes, 30 minutes, 40 minutes, 50 minutes, 60 minutes, 70 minutes, 80 minutes, 90 minutes, 100 minutes, 110 minutes, 120 minutes, 150 minutes, 180 minutes, or more.

For example, a pharmaceutical formulation comprising an anti-IL-4R antibody may be deemed stable if after nine months of storage at 5° C., less than about 2%, 1.75%, 1.5%, 1.25%, 1%, 0.75%, 0.5%, 0.25%, or 0.1% of the antibody is detected in an aggregated form. A pharmaceutical formulation may also be deemed stable if after 56 days of storage at 45° C., less than about 12% of the protein is detected in an aggregated form. A pharmaceutical formulation may also be deemed stable if after 42 days of storage at 45° C., less than about 10% or less than about 9% of the protein is detected in an aggregated form. A pharmaceutical formulation may also be deemed stable if after 28 days of storage at 45° C., less than about 8% or less than about 7.5% or less than about 7% of the protein is detected in an aggregated form. A pharmaceutical formulation may also be deemed stable if after 14 days of storage at 45° C., less than about 6% of the protein is detected in an aggregated form. A pharmaceutical formulation may also be deemed stable if after three months of storage at −20° C., −30° C., or −80° C. less than about 2%, 1.9%, 1.8%, 1.7%, 1.6%, 1.5%, 1%, 0.5%, or 0.1% of the protein is detected in an aggregated form. A pharmaceutical formulation may also be deemed stable if after 120 minutes of agitation (e.g., via vortexing) at room temperature less than 3% or less than 2.5% of the protein is detected in an aggregated form.

Stability can also be measured, inter alia, by determining particulate formulation within the formulation after storage for a defined amount of time at a defined temperature. Particle formation can be determined, for example, by microscopy techniques or by micro-flow imaging techniques.

In some aspects, the formulations of the present disclosure comprise a detectable amount of a lipase (for example, PLBL2). Methods of detecting and quantifying the presence and activity of phospholipase are known in the art. In some aspects, the phospholipase is detected by immunoassay (e.g., ELISA). In some aspects, the phospholipase is detected by liquid chromatography-mass spectrometry (LC-MS).

Accordingly, a pharmaceutical formulation (containing an esterase or lipase) may be deemed stable if after storage for a period of time (e.g., 6, 12, 18, 24 or 36 months or more) at a defined temperature (e.g., 5° C.), no more than a specified number of fatty acid particles ≥10 μm or ≥25 μm in size (e.g., 3000 particles, 1000 particles, 500 particles, 250 particles, 100 particles, or 50 particles) are identified within a volume of 2.25 mL. For example, a pharmaceutical formulation may be deemed stable if after 24 months of storage at 5° C. no more than 3000 fatty acid particles are identified within a volume of 2.25 mL via microscopy. In another aspect, a pharmaceutical formulation may be deemed stable if after 24 months of storage at 5° C. no more than 1000 fatty acid particles are identified within a volume of 2.25 mL via microscopy. A pharmaceutical formulation may also be deemed stable if after 24 months of storage at 5° C. no more than 500 fatty acid particles are identified within a volume of 2.25 mL via microscopy. A pharmaceutical formulation may also be deemed stable if after 24 months of storage at 5° C. no more than 250 fatty acid particles are identified within a volume of 2.25 mL via microscopy. A pharmaceutical formulation may also be deemed stable if after 24 months of storage at 5° C. no more than 150 fatty acid particles are identified within a volume of 2.25 mL via microscopy. A pharmaceutical formulation may also be deemed stable if after 36 months of storage at 5° C. no more than 1000 fatty acid particles are identified within a volume of 2.25 mL via microscopy. A pharmaceutical formulation may also be deemed stable if after 36 months of storage at 5° C. no more than 500 fatty acid particles are identified within a volume of 2.25 mL via microscopy. A pharmaceutical formulation may also be deemed stable if after 36 months of storage at 5° C. no more than 250 fatty acid particles are identified within a volume of 2.25 mL via microscopy. A pharmaceutical formulation may also be deemed stable if after 36 months of storage at 5° C. no more than 150 fatty acid particles are identified within a volume of 2.25 mL via microscopy. A pharmaceutical formulation may also be deemed stable if after 36 months of storage at 5° C. no more than 100 fatty acid particles are identified within a volume of 2.25 mL via microscopy. A pharmaceutical formulation may also be deemed stable if after 36 months of storage at 5° C. no more than 50 fatty acid particles are identified within a volume of 2.25 mL via microscopy.

Stability can also be measured by, inter alia, determining the percentage of native protein of interest remaining in the formulation after storage for a defined amount of time at a given temperature. The percentage of native protein of interest can be determined by, inter alia, size exclusion chromatography (e.g., size exclusion high performance liquid chromatography (SE-HPLC)). An “acceptable degree of stability,” as that phrase is used herein, means that at least 90% of the native form of the protein can be detected in the formulation after storage for a defined amount of time at a given temperature. In certain aspects, at least about 90%, 91%, 92%, 93%, 94%, 95%, 96%, 97%, 98%, 99% or 100% of the native form of the protein can be detected in the formulation after storage for a defined amount of time at a given temperature. The defined amount of time after which stability is measured can be at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 4° C.-8° C., about 5° C., about 25° C., about 35° C., about 37° C., or about 45° C.

Stability can also be measured, inter alia, by determining the percentage of protein of interest that migrates in a more acidic fraction during ion exchange (“acidic form”) than in the main fraction of protein (“main charge form”), wherein stability is inversely proportional to the fraction of protein in the acidic form. Deamidation of the protein may cause the protein to become more negatively charged and thus more acidic relative to the non-deamidated protein (see, e.g., Robinson, N., Protein Deamidation, PNAS, Apr. 16, 2002, 99 (8): 5283-5288). The percentage of “acidified” protein can be determined by ion exchange chromatography (e.g., cation exchange high performance liquid chromatography (CEX-HPLC) or cation exchange ultra-performance liquid chromatography (CEX-UPLC)). An “acceptable degree of stability”, as that phrase is used herein, means that at most 50%, 45%, 40%, 35%, 30%, 25%, 20%, 15%, 10%, 5%, 4%, 3%, 2%, 1%, 0.5%, or 0.1% of the protein can be detected in an acidic form in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 4° C.-8° C., about 5° C., about 25° C., or about 45° C.

Measuring the binding affinity of an antibody of interest to its target may also be used to assess stability. For example, a formulation of the present disclosure may be regarded as stable if, after storage at e.g., −80° C., −30° C., −20° C., 5° C., 25° C., 37° C., 45° C., etc. for a defined amount of time (e.g., 14 days to 9 months), an anti-IL-4R antibody contained within the formulation binds to hIL-4Rα with an affinity that is at least 80%, 85%, 90%, 95%, or more of the binding affinity of the antibody prior to said storage. Binding affinity may be determined by any method, such as e.g., ELISA or plasmon resonance. Biological activity may be determined by, for example, measuring the downstream activity of the IL-4R system in the presence of the antibody, and comparing the activity to the activity of the IL-4R system in the absence of antibody.

References to stability of the pharmaceutical formulations “after” a specified period of time are intended to mean that a measurement of a stability parameter (e.g., % native form, % HMW species, or % acidic form) is taken at or about the end of the specific time period, and is not intended to mean that the pharmaceutical formulation necessarily maintains the same degree of stability for the measured parameter thereafter. For example, reference to a particular stability after 12 months means that the measurement of stability was taken at or about 12 months after the start of the study.

### Containers and Methods of Administration

The pharmaceutical formulations of the present disclosure may be contained within any container suitable for storage of medicines and other therapeutic compositions. For example, the pharmaceutical formulations may be contained within a sealed and sterilized plastic or glass container having a defined volume such as a vial, ampule, syringe, cartridge, bottle or IV bag. Different types of vials can be used to contain the formulations of the present disclosure including, for example, clear and opaque (e.g., amber) glass or plastic vials. Likewise, any type of syringe can be used to contain and/or administer the pharmaceutical formulations of the present disclosure. In some aspects, the pharmaceutical formulation is contained in a prefilled syringe (PFS). In some aspects, the pharmaceutical formulation is contained in a prefilled staked needle syringe.

The pharmaceutical formulations of the present disclosure may be contained within “normal tungsten” syringes or “low tungsten” syringes. As will be appreciated by persons of ordinary skill in the art, the process of making glass syringes generally involves the use of a hot tungsten rod which functions to pierce the glass thereby creating a hole from which liquids can be drawn and expelled from the syringe. This process results in the deposition of trace amounts of tungsten on the interior surface of the syringe. Subsequent washing and other processing steps can be used to reduce the amount of tungsten in the syringe. As used herein, the term “normal tungsten” means that the syringe contains greater than 500 parts per billion (ppb) of tungsten. The term “low tungsten” means that the syringe contains less than 500 ppb of tungsten. For example, a low tungsten syringe, according to the present disclosure, can contain less than about 490, 480, 470, 460, 450, 440, 430, 420, 410, 390, 350, 300, 250, 200, 150, 100, 90, 80, 70, 60, 50, 40, 30, 20, 10 or fewer ppb of tungsten.

The rubber plungers used in syringes, and the rubber stoppers used to close the openings of vials, may be coated to prevent contamination of the medicinal contents of the syringe or vial and/or to preserve their stability. Thus, pharmaceutical formulations of the present disclosure, according to certain aspects, may be contained within a syringe that comprises a coated plunger, or within a vial that is sealed with a coated rubber stopper. For example, the plunger or stopper may be coated with a fluorocarbon film. Examples of coated stoppers and/or plungers suitable for use with vials and syringes containing the pharmaceutical formulations of the present disclosure are mentioned in, for example, U.S. Pat. Nos. 4,997,423; 5,908,686; 6,286,699; 6,645,635; and 7,226,554, the contents of which are incorporated by reference herein in their entireties. Particular exemplary coated rubber stoppers and plungers that can be used in the context of the present disclosure are commercially available under the tradename “FluroTec®,” available from West Pharmaceutical Services, Inc. (Lionville, PA). According to certain aspects of the present disclosure, the pharmaceutical formulations may be contained within a low tungsten syringe that comprises a fluorocarbon-coated plunger. In some aspects, the container is a syringe, such as an Ompi EZ-Fill™ syringe or a BD Neopak™ syringe. In some cases, the syringe is a 1 mL long glass syringe with a 1 mL iWest piston, a 27G thin wall needle and an FM30 needle shield or a BD260 needle shield. In some cases, the syringe is a 2.25 mL glass syringe (e.g., Nuova Ompi). In various aspects, the syringe is a 0.5 mL, 0.6 mL, 0.7 mL, 0.8 mL, 0.9 mL, 1.0 mL, 1.1 mL, 1.2 mL, 1.3 mL, 1.4 mL, 1.5 mL, 1.6 mL, 1.7 mL, 1.8 mL, 1.9 mL, 2.0 mL, 2.1 mL, 2.2 mL, 2.3 mL, 2.4 mL, 2.5 mL, 2.6 mL, 2.7 mL, 2.8 mL, 2.9 mL, 3.0 mL, 3.5 mL, 4.0 mL, 4.5 mL, 5.0 mL, 5.5 mL, 6.0 mL, 6.5 mL, 7.0 mL, 7.5 mL, 8.0 mL, 8.5 mL, 9.0 mL, 9.5 mL, or 10 ml syringe (e.g., a glass syringe).

The pharmaceutical formulations can be administered to a patient by parenteral routes such as injection (e.g., subcutaneous, intravenous, intramuscular, intraperitoneal, etc.) or percutaneous, mucosal, nasal, pulmonary and/or oral administration. Numerous reusable pen and/or autoinjector delivery devices can be used to subcutaneously deliver the pharmaceutical formulations of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen and/or autoinjector delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park, IL), to name only a few. In some cases, the pharmaceutical formulation is contained in a syringe specifically adapted for use with an autoinjector. Subcutaneous injections may be administered using a 20-30 gauge needle, or a 25-30 gauge needle. In some cases, subcutaneous injections may be administered using a 25 gauge needle. In some cases, subcutaneous injections may be administered using a 27 gauge needle. In some cases, subcutaneous injections may be administered using a 29 gauge needle.

Another type of delivery device can include a safety system. Such devices can be relatively inexpensive, and operate to manually or automatically extend a safety sleeve over a needle once injection is complete. Examples of safety systems can include the ERIS device by West Pharmaceutical, or the UltraSafe device by Becton Dickinson. In addition, the use of a large volume device (“LVD”), or bolus injector, to deliver the pharmaceutical formulations of the present disclosure is also contemplated herein. In some cases, the LVD or bolus injector may be configured to inject a medicament into a patient. For example, an LVD or bolus injector may be configured to deliver a “large” volume of medicament (typically about 2 mL to about 10 mL).

The pharmaceutical formulations of the present disclosure can also be contained in a unit dosage form. The term “unit dosage form,” as used herein, refers to a physically discrete unit suitable as a unitary dosage for the patient to be treated, each unit containing a predetermined quantity of active compound calculated to produce the desired therapeutic effect in association with the required pharmaceutical carrier, diluent, or excipient. In various aspects, the unit dosage form is contained within a container as discussed herein. Actual dosage levels of the active ingredient (for example, an anti-IL-4R antibody) in the formulations of the present disclosure may be varied so as to obtain an amount of the active ingredient which is effective to achieve the desired therapeutic response for a particular patient, composition, and mode of administration, without adverse effect to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. The term “diluent” as used herein refers to a solution suitable for altering or achieving an exemplary or appropriate concentration or concentrations as described herein.

In various aspects, the unit dosage form contains an amount of the active ingredient (for example, an anti-IL-4R antibody) intended for a single use. In various aspects, the amount of the active ingredient in the unit dosage form is from about 0.1 mg to about 5000 mg, from about 100 mg to about 1000 mg, and from about 100 mg to about 500 mg, from about 100 mg to about 400 mg, from about 100 mg to about 200 mg, from about 250 mg to about 350 mg, from about 125 mg to about 175 mg, from about 275 mg to about 325 mg, or ranges or intervals thereof. For example, ranges of values using a combination of any of the above recited values (or values contained within the above recited ranges) as upper and/or lower limits are intended to be included. In a particular aspect, the formulation often is supplied as a liquid in unit dosage form. In some aspects, the unit dosage form contains about 100 mg of the active ingredient. In some aspects, the unit dosage form contains about 150 mg. In some aspects, the unit dosage form contains about 200 mg. In some aspects, the unit dosage form contains about 300 mg. In some aspects, the unit dosage form contains about 350 mg. In some aspects, the unit dosage form contains about 600 mg. In some aspects, a unit dosage form according to the present disclosure is suitable for subcutaneous administration to a patient.

The present disclosure also includes methods of preparing a unit dosage form. In one aspect, a method for preparing a pharmaceutical unit dosage form includes combining the formulation of any of foregoing aspects in a suitable container (e.g., those containers discussed herein).

### Therapeutic Uses of the Pharmaceutical Formulations

Pharmaceutical formulations of the present disclosure comprising an anti-IL-4R antibody are useful, inter alia, for the treatment, prevention and/or amelioration of any disease or disorder associated with IL-4R activity.

The therapeutic methods of the present disclosure comprise administering to a subject any formulation comprising an anti-hIL-4R antibody as disclosed herein. The subject to which the pharmaceutical formulation is administered can be, e.g., any human or non-human animal that is in need of such treatment, prevention and/or amelioration, or who would otherwise benefit from the inhibition or attenuation of IL-4R and/or IL-4R-mediated activity. The present disclosure further includes the use of any of the pharmaceutical formulations disclosed herein in the manufacture of a medicament for the treatment, prevention and/or amelioration of any disease or disorder associated with IL-4R activity.

In some aspects, the disease or disorder associated with IL-4R activity is an inflammatory condition, allergic condition, lung/respiratory disorder, gastrointestinal disorder, or dermatological disorder. In some aspects, the disease or disorder is a Type 2 inflammatory disorder. In some aspects, the disease or disorder is an atopic disease. Non-limiting examples of diseases and disorders associated with IL-4R activity include allergy (e.g., food allergy, environmental allergy, grass allergy, peanut allergy, dairy allergy), allergic reactions, allergic bronchopulmonary aspergillosis, allergic fungal rhino-sinusitis (AFRS), allergic rhinitis, alopecia areata, asthma (including mild, moderate, or severe asthma or persistent asthma), arthritis (including septic arthritis), atopic dermatitis (including moderate or severe atopic dermatitis), hand and foot atopic dermatitis, atopic keratoconjunctivitis, autoimmune hemolytic anemia, autoimmune lymphoproliferative syndrome, autoimmune uveitis, Barrett's esophagus, benign prostate hyperplasia, bronchiectasis, bullous pemphigoid, Churg-Strauss syndrome, chronic idiopathic urticaria, cold inducible urticaria, chronic inducible urticaria, chronic spontaneous urticaria (CSU), contact dermatitis (e.g., allergic contact dermatitis), chronic obstructive pulmonary disease (COPD), eosinophilic esophagitis, eosinophilic gastroenteritis, Grave's disease, herpetiformis, hypertrophic scarring, inflammatory bowel disease, Kawasaki disease, nasal polyposis, nephrosis, Netherton's syndrome, pre-eclampsia, prurigo nodularis, pruritus (e.g., chronic pruritus of unknown origin), rhinitis (e.g., allergic rhinitis), rhinosinusitis (e.g., allergic fungal rhinosinusitis, chronic rhinosinusitis with or without nasal polyposis), scleroderma, sickle cell disease, Sjogren's syndrome, tuberculosis, ulcerative colitis, and Whipple's Disease.

In some aspects, the present disclosure provides kits comprising a pharmaceutical formulation (e.g., a container with the formulation or a unit dosage form), as discussed herein, and packaging or labeling (e.g., a package insert) with instructions to use the pharmaceutical formulation for the treatment of a disease or disorder, as discussed above. In some cases, the instructions provide for use of a unit dosage form, as discussed herein, for the treatment of a disease or disorder.

A summary of the sequences and the corresponding SEQ ID NOs referenced herein is shown in Table 1, below.

TABLE 1
Informal Sequence Listing

| SEQ | | |
|---|---|---|
| ID NO | Sequence | Description |
| 1 | EVQLVESGGGLEQPGGSLRLSCAGSGFTFRDYAMTWVRQAPGKGLE | Dupilumab HCVR |
| | WVSSISGSGGNTYYADSVKGRFTISRDNSKNTLYLQMNSLRAEDTA | amino acid sequence |
| | VYYCAKDRLSITIRPRYYGLDVWGQGTTVTVS | |
| 2 | DIVMTQSPLSLPVTPGEPASISCRSSQSLLYSIGYNYLDWYLQKSGQS | Dupilumab LCVR |
| | PQLLIYLGSNRASGVPDRFSGSGSGTDFTLKISRVEAEDVGFYYCMQ | amino acid sequence |
| | ALQTPYTFGQGTKLEIK | |
| 3 | GFTFRDYA | Dupilumab HCDR1 |
| | | amino acid sequence |
| 4 | ISGSGGNT | Dupilumab HCDR2 |
| | | amino acid sequence |
| 5 | AKDRLSITIRPRYYGLDV | Dupilumab HCDR3 |
| | | amino acid sequence |
| 6 | QSLLYSIGYNY | Dupilumab LCDR1 |
| | | amino acid sequence |
| 7 | LGS | Dupilumab LCDR2 |
| | | amino acid sequence |
| 8 | MQALQTPYT | Dupilumab LCDR3 |
| | | amino acid sequence |
| 9 | EVQLVESGGGLEQPGGSLRLSCAGSGFTFRDYAMTWVRQAPGKGLE | Dupilumab heavy chain |
| | WVSSISGSGGNTYYADSVKGRFTISRDNSKNTLYLQMNSLRAEDTA | amino acid sequence |
| | VYYCAKDRLSITIRPRYYGLDVWGQGTTVTVSSASTKGPSVFPLAPC | |
| | SRSTSESTAALGCLVKDYFPEPVTVSWNSGALTSGVHTFPAVLQSSG | |
| | LYSLSSVVTVPSSSLGTKTYTCNVDHKPSNTKVDKRVESKYGPPCPP | |
| | CPAPEFLGGPSVFLFPPKPKDTLMISRTPEVTCVVVDVSQEDPEVQFN | |
| | WYVDGVEVHNAKTKPREEQFNSTYRVVSVLTVLHQDWLNGKEYK | |
| | CKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKS | |
| | RWQEGNVFSCSVMHEALHNHYTQKSLSLSLGK | |
| 10 | DIVMTQSPLSLPVTPGEPASISCRSSQSLLYSIGYNYLDWYLQKSGQS | Dupilumab light chain |
| | PQLLIYLGSNRASGVPDRFSGSGSGTDFTLKISRVEAEDVGFYYCMQ | amino acid sequence |
| | ALQTPYTFGQGTKLEIKRTVAAPSVFIFPPSDEQLKSGTASVVCLLNN | |
| | FYPREAKVQWKVDNALQSGNSQESVTEQDSKDSTYSLSSTLTLSKA | |
| | DYEKHKVYACEVTHQGLSSPVTKSFNRGEC | |
| 11 | MKVLQEPTCVSDYMSISTCEWKMNGPTNCSTELRLLYQLVFLLSEA | Human IL-4Ra |
| | HTCIPENNGGAGCVCHLLMDDVVSADNYTLDLWAGQQLLWKGSF | |
| | KPSEHVKPRAPGNLTVHTNVSDTLLLTWSNPYPPDNYLYNHLTYAV | |
| | NIWSENDPADFRIYNVTYLEPSLRIAASTLKSGISYRARVRAWAQCY | |
| | NTTWSEWSPSTKWHNSYREPFEQH | |
| 12 | EIVLTQSPGTLSLSPGERATLSCRASQSVSNSYLAWYQQKPGQAPRL | SCB-VL-39 |
| | LIFGASSRATGIPDRFSGSGSGTDFTLTISRLEPEDFAVYYCQQYGSSP | |
| | PWTFGQGTKVEIK | |
| 13 | EIVLTQSPGTLSLSPGERATLSCRASQSVSSSYLAWYQQKPGQAPRLL | SCB-VL-40 |
| | IYGASSRATGIPDRFSGSGSGTDFTLTISRLEPEDFAVYYCQQYGSSPP | |
| | WTFGQGTKVEIK | |
| 14 | EIVLTQSPGTLSLSPGERATLSCRASQSVSSSYLAWYQQKPGQAPRLL | SCB-VL-41 |
| | IFGASSRAPGIPDRFSGSGSGTDFTLTISRLEPEDFAVYYCQQYGSSPP | |
| | WTFGQGTKVEIK | |
| 15 | EIVLTQSPGTLSLSPGERATLSCRASQSVSNSYLAWYQQKPGQAPRL | SCB-VL-42 |
| | LIYGASSRATGIPDRFSGSGSGTDFTLTISRLEPEDFAVYYCQQYGSSP | |
| | PWTFGQGTKVEIK | |
| 16 | EIVLTQSPGTLSLSPGERATLSCRASQSVSNSYLAWYQQKPGQAPRL | SCB-VL-43 |
| | LIFGASSRAPGIPDRFSGSGSGTDFTLTISRLEPEDFAVYYCQQYGSSP | |
| | PWTFGQGTKVEIK | |
| 17 | EIVLTQSPGTLSLSPGERATLSCRASQSVSSSYLAWYQQKPGQAPRLL | SCB-VL-44 |
| | IYGASSRAPGIPDRFSGSGSGTDFTLTISRLEPEDFAVYYCQQYGSSPP | |
| | WTFGQGTKVEIK | |
| 18 | EIVLTQSPGTLSLSPGERATLSCRASQSVSSSYLAWYQQKPGQAPRLL | SCB-VL-45 |
| | IFGASSRATGIPDRFSGSGSGTDFTLTISRLEPEDFAVYYCQQYDHSPP | |
| | WTFGQGTKVEIK | |
| 19 | EIVLTQSPGTLSLSPGERATLSCRASQSVSSSYLAWYQQKPGQAPRLL | SCB-VL-46 |
| | IFGASSRATGIPDRFSGSGSGTDFTLTISRLEPEDFAVYYCQQYGSSAG | |
| | WTFGQGTKVEIK | |
| 20 | EIVLTQSPGTLSLSPGERATLSCRASQSVSSSYLAWYQQKPGQAPRLL | SCB-VL-47 |
| | IFGASSRATGIPDRFSGSGSGTDFTLTISRLEPEDFAVYYCQQYDHSA | |
| | GWTFGQGTKVEIK | |
| 21 | EIVLTQSPGTLSLSPGERATLSCRASQSVSNSYLAWYQQKPGQAPRL | SCB-VL-48 |
| | LIFGASSRATGIPDRFSGSGSGTDFTLTISRLEPEDFAVYYCQQYDHSP | |
| | PWTFGQGTKVEIK | |
| 22 | EIVLTQSPGTLSLSPGERATLSCRASQSVSSSYLAWYQQKPGQAPRLL | SCB-VL-49 |
| | IYGASSRATGIPDRFSGSGSGTDFTLTISRLEPEDFAVYYCQQYDHSPP | |
| | WTFGQGTKVEIK | |
| 23 | EIVLTQSPGTLSLSPGERATLSCRASQSVSSSYLAWYQQKPGQAPRLL | SCB-VL-50 |
| | IFGASSRAPGIPDRFSGSGSGTDFTLTISRLEPEDFAVYYCQQYDHSPP | |
| | WTFGQGTKVEIK | |
| 24 | EIVLTQSPGTLSLSPGERATLSCRASQSVSSSYLAWYQQKPGQAPRLL | SCB-VL-51 |
| | IYGASSRAPGIPDRFSGSGSGTDFTLTISRLEPEDFAVYYCQQYDHSA | |
| | GWTFGQGTKVEIK | |
| 25 | EIVLTQSPGTLSLSPGERATLSCRASQSVSNSYLAWYQQKPGQAPRL | SCB-VL-52 |
| | LIFGASSRAPGIPDRFSGSGSGTDFTLTISRLEPEDFAVYYCQQYDHSA | |
| | GWTFGQGTKVEIK | |
| 26 | EIVLTQSPGTLSLSPGERATLSCRASQSVSNSYLAWYQQKPGQAPRL | SCB-VL-53 |
| | LIYGASSRATGIPDRFSGSGSGTDFTLTISRLEPEDFAVYYCQQYDHS | |
| | AGWTFGQGTKVEIK | |
| 27 | EIVLTQSPGTLSLSPGERATLSCRASQSVSSSYLAWYQQKPGQAPRLL | SCB-VL-54 |
| | IFGASSRAPGIPDRFSGSGSGTDFTLTISRLEPEDFAVYYCQQYDHSA | |
| | GWTFGQGTKVEIK | |
| 28 | EIVLTQSPGTLSLSPGERATLSCRASQSVSSSYLAWYQQKPGQAPRLL | SCB-VL-55 |
| | IYGASSRATGIPDRFSGSGSGTDFTLTISRLEPEDFAVYYCQQYDHSA | |
| | GWTFGQGTKVEIK | |
| 29 | EIVLTQSPGTLSLSPGERATLSCRASQSVSNSYLAWYQQKPGQAPRL | SCB-VL-56 |
| | LIFGASSRATGIPDRFSGSGSGTDFTLTISRLEPEDFAVYYCQQYDHSA | |
| | GWTFGQGTKVEIK | |
| 30 | EIVLTQSPGTLSLSPGERATLSCRASQSVSSSYLAWYQQKPGQAPRLL | SCB-VL-57 |
| | IFGASSRATGIPDRFSGSGSGTDFTLTISRLEPEDFAVYYCQQYGSSPP | |
| | WTFGQGTKVEIK | |
| 31 | EIVLTQSPGTLSLSPGERATLSCRASQSVSNSYLAWYQQKPGQAPRL | SCB-VL-58 |
| | LIYGASSRAPGIPDRFSGSGSGTDFTLTISRLEPEDFAVYYCQQYDHS | |
| | AGWTFGQGTKVEIK | |
| 32 | EVQLVESGGGLVHPGGSLRLSCAGSGFTFSRNAMFWVRQAPGKGLE | SCB-VH-59 |
| | WVSGIGTGGATNYADSVKGRFTISRDNAKNSLYLQMNSLRAEDMA | |
| | VYYCARGRYYFDYWGQGTLVTVSS | |
| 33 | EVQLVQSGGGLVQPGGSLRLSCAGSGFTFSRNAMFWVRQAPGKGLE | SCB-VH-60 |
| | WVSGIGTGGATNYADSVKGRFTISRDNAKNSLYLQMNSLRAEDMA | |
| | VYYCARGRYYFDYWGQGTLVTVSS | |
| 34 | EVQLVQSGGGLVHPGGSLRLSCAASGFTFSRNAMFWVRQAPGKGLE | SCB-VH-61 |
| | WVSGIGTGGATNYADSVKGRFTISRDNAKNSLYLQMNSLRAEDMA | |
| | VYYCARGRYYFDYWGQGTLVTVSS | |
| 35 | EVQLVQSGGGLVHPGGSLRLSCAGSGFTFSRNAMFWVRQAPGKGLE | SCB-VH-62 |
| | WVSGIGTGGATSYADSVKGRFTISRDNAKNSLYLQMNSLRAEDMA | |
| | VYYCARGRYYFDYWGQGTLVTVSS | |
| 36 | EVQLVQSGGGLVHPGGSLRLSCAGSGFTFSRNAMFWVRQAPGKGLE | SCB-VH-63 |
| | WVSGIGTGGATNYADSVKGRFTISRDNAKNSLYLQMNSLRAEDTAV | |
| | YYCARGRYYFDYWGQGTLVTVSS | |
| 37 | EVQLVESGGGLVQPGGSLRLSCAGSGFTFSRNAMFWVRQAPGKGLE | SCB-VH-64 |
| | WVSGIGTGGATNYADSVKGRFTISRDNAKNSLYLQMNSLRAEDMA | |
| | VYYCARGRYYFDYWGQGTLVTVSS | |
| 38 | EVQLVESGGGLVHPGGSLRLSCAASGFTFSRNAMFWVRQAPGKGLE | SCB-VH-65 |
| | WVSGIGTGGATNYADSVKGRFTISRDNAKNSLYLQMNSLRAEDMA | |
| | VYYCARGRYYFDYWGQGTLVTVSS | |
| 39 | EVQLVQSGGGLVQPGGSLRLSCAASGFTFSRNAMFWVRQAPGKGLE | SCB-VH-66 |
| | WVSGIGTGGATNYADSVKGRFTISRDNAKNSLYLQMNSLRAEDMA | |
| | VYYCARGRYYFDYWGQGTLVTVSS | |
| 40 | EVQLVQSGGGLVHPGGSLRLSCAGSGFTFSRNAMFWVRQAPGKGLE | SCB-VH-67 |
| | WVSGIGTGGATSYADSVKGRFTISRDNAKNSLYLQMNSLRAEDTAV | |
| | YYCARGRYYFDYWGQGTLVTVSS | |
| 41 | EVQLVQSGGGLVHPGGSLRLSCAGSGFTFSRNAMFWVRQAPGKGLE | SCB-VH-68 |
| | WVSGIGTGGATNYADSVKGRFTISRDNAKNSLYLQMNSLRAEDMA | |
| | VYYCARGRYYFPWWGQGTLVTVSS | |
| 42 | EVQLVESGGGLVHPGGSLRLSCAGSGFTFSRNAMFWVRQAPGKGLE | SCB-VH-69 |
| | WVSGIGTGGATNYADSVKGRFTISRDNAKNSLYLQMNSLRAEDMA | |
| | VYYCARGRYYFPWWGQGTLVTVSS | |
| 43 | EVQLVQSGGGLVQPGGSLRLSCAGSGFTFSRNAMFWVRQAPGKGLE | SCB-VH-70 |
| | WVSGIGTGGATNYADSVKGRFTISRDNAKNSLYLQMNSLRAEDMA | |
| | VYYCARGRYYFPWWGQGTLVTVSS | |
| 44 | EVQLVQSGGGLVHPGGSLRLSCAASGFTFSRNAMFWVRQAPGKGLE | SCB-VH-71 |
| | WVSGIGTGGATNYADSVKGRFTISRDNAKNSLYLQMNSLRAEDMA | |
| | VYYCARGRYYFPWWGQGTLVTVSS | |
| 45 | EVQLVQSGGGLVHPGGSLRLSCAGSGFTFSRNAMFWVRQAPGKGLE | SCB-VH-72 |
| | WVSGIGTGGATSYADSVKGRFTISRDNAKNSLYLQMNSLRAEDMA | |
| | VYYCARGRYYFPWWGQGTLVTVSS | |
| 46 | EVQLVQSGGGLVHPGGSLRLSCAGSGFTFSRNAMFWVRQAPGKGLE | SCB-VH-73 |
| | WVSGIGTGGATNYADSVKGRFTISRDNAKNSLYLQMNSLRAEDTAV | |
| | YYCARGRYYFPWWGQGTLVTVSS | |
| 47 | EVQLVQSGGGLVHPGRSLRLSCAGSGFTFSRNAMFWVRQAPGKGLE | SCB-VH-74 |
| | WVSGIGTGGATNYADSVKGRFTISRDNAKNSLYLQMNSLRAEDMA | |
| | VYYCARGRYYFDYWGQGTLVTVSS | |
| 48 | EVQLVQSGGGLVHPGGSLRLTCAGSGFTFSRNAMFWVRQAPGKGL | SCB-VH-75 |
| | EWVSGIGTGGATNYADSVKGRFTISRDNAKNSLYLQMNSLRAEDM | |
| | AVYYCARGRYYFDYWGQGTLVTVSS | |
| 49 | EVQLVQSGGGLVHPGGSLRLSCAGSGFTFSRNAMHWVRQAPGKGL | SCB-VH-76 |
| | EWVSGIGTGGATNYADSVKGRFTISRDNAKNSLYLQMNSLRAEDM | |
| | AVYYCARGRYYFDYWGQGTLVTVSS | |
| 50 | EVQLVQSGGGLVHPGGSLRLSCAGSGFTFSRNAMFWVRQAPGEGLE | SCB-VH-77 |
| | WVSGIGTGGATNYADSVKGRFTISRDNAKNSLYLQMNSLRAEDMA | |
| | VYYCARGRYYFDYWGQGTLVTVSS | |
| 51 | EVQLVQSGGGLVHPGGSLRLSCAGSGFTFSRNAMFWVRQAPGKGLE | SCB-VH-78 |
| | WVSGIGTGGATNYADSVKGRFTISRDEAKNSLYLQMNSLRAEDMA | |
| | VYYCARGRYYFDYWGQGTLVTVSS | |
| 52 | EVQLVQSGGGLVHPGGSLRLSCAGSGFTFSRNAMFWVRQAPGKGLE | SCB-VH-79 |
| | WVSGIGTGGATNYADSVKGRFTISRDNAKNSLYLQMNSLRAGDMA | |
| | VYYCARGRYYFDYWGQGTLVTVSS | |
| 53 | EVQLVQSGGGLVHPGGSLRLSCAGSGFTFDDYAMFWVRQAPGKGL | SCB-VH-80 |
| | EWVSGIGTGGATNYADSVKGRFTISRDNAKNSLYLQMNSLRAEDM | |
| | AVYYCARGRYYFDYWGQGTLVTVSS | |
| 54 | EVQLVQSGGGLVQPGGSLRLSCAASGFTFSRNAMFWVRQAPGKGLE | SCB-VH-81 |
| | WVSGIGTGGATSYADSVKGRFTISRDNAKNSLYLQMNSLRAEDTAV | |
| | YYCARGRYYFPWWGQGTLVTVSS | |
| 55 | EVQLVESGGGLVHPGGSLRLSCAASGFTFSRNAMFWVRQAPGKGLE | SCB-VH-82 |
| | WVSGIGTGGATSYADSVKGRFTISRDNAKNSLYLQMNSLRAEDTAV | |
| | YYCARGRYYFPWWGQGTLVTVSS | |
| 56 | EVQLVESGGGLVQPGGSLRLSCAGSGFTFSRNAMFWVRQAPGKGLE | SCB-VH-83 |
| | WVSGIGTGGATSYADSVKGRFTISRDNAKNSLYLQMNSLRAEDTAV | |
| | YYCARGRYYFPWWGQGTLVTVSS | |
| 57 | EVQLVESGGGLVQPGGSLRLSCAASGFTFSRNAMFWVRQAPGKGLE | SCB-VH-84 |
| | WVSGIGTGGATNYADSVKGRFTISRDNAKNSLYLQMNSLRAEDTAV | |
| | YYCARGRYYFPWWGQGTLVTVSS | |
| 58 | EVQLVESGGGLVQPGGSLRLSCAASGFTFSRNAMFWVRQAPGKGLE | SCB-VH-85 |
| | WVSGIGTGGATSYADSVKGRFTISRDNAKNSLYLQMNSLRAEDMA | |
| | VYYCARGRYYFPWWGQGTLVTVSS | |
| 59 | EVQLVQSGGGLVHPGGSLRLSCAASGFTFSRNAMFWVRQAPGKGLE | SCB-VH-86 |
| | WVSGIGTGGATSYADSVKGRFTISRDNAKNSLYLQMNSLRAEDTAV | |
| | YYCARGRYYFPWWGQGTLVTVSS | |
| 60 | EVQLVQSGGGLVQPGGSLRLSCAGSGFTFSRNAMFWVRQAPGKGLE | SCB-VH-87 |
| | WVSGIGTGGATSYADSVKGRFTISRDNAKNSLYLQMNSLRAEDTAV | |
| | YYCARGRYYFPWWGQGTLVTVSS | |
| 61 | EVQLVESGGGLVHPGGSLRLSCAGSGFTFSRNAMFWVRQAPGKGLE | SCB-VH-88 |
| | WVSGIGTGGATSYADSVKGRFTISRDNAKNSLYLQMNSLRAEDTAV | |
| | YYCARGRYYFPWWGQGTLVTVSS | |
| 62 | EVQLVQSGGGLVHPGGSLRLSCAGSGFTFSRNAMFWVRQAPGKGLE | SCB-VH-89 |
| | WVSGIGTGGATSYADSVKGRFTISRDNAKNSLYLQMNSLRAEDTAV | |
| | YYCARGRYYFPWWGQGTLVTVSS | |
| 63 | EVQLVESGGGLVQPGGSLRLSCAASGFTFSRNAMFWVRQAPGKGLE | SCB-VH-90 |
| | WVSGIGTGGATNYADSVKGRFTISRDNAKNSLYLQMNSLRAEDMA | |
| | VYYCARGRYYFPWWGQGTLVTVSS | |
| 64 | EVQLVESGGGLVQPGGSLRLSCAASGFTFSRNAMFWVRQAPGKGLE | SCB-VH-91 |
| | WVSGIGTGGATSYADSVKGRFTISRDNAKNSLYLQMNSLRAEDTAV | |
| | YYCARGRYYFDYWGQGTLVTVSS | |
| 65 | EVQLVQSGGGLVHPGGSLRLSCAGSGFTFSRNAMFWVRQAPGKGLE | SCB-VH-92 |
| | WVSGIGTGGATNYADSVKGRFTISRDNAKNSLYLQMNSLRAEDMA | |
| | VYYCARGRYYFDYWGQGTLVTVSS | |
| 66 | EVQLVESGGGLVQPGGSLRLSCAASGFTFSRNAMFWVRQAPGKGLE | SCB-VH-93 |
| | WVSGIGTGGATSYADSVKGRFTISRDNAKNSLYLQMNSLRAEDTAV | |
| | YYCARGRYYFPWWGQGTLVTVSS | |
| 67 | QVQLVQSGAEVKKPGASVKVSCKASGYAFTSYYMHWARQAPGQG | MEDI-1-VH |
| | LEWMGIINPSGGSTSYAQKFQGRVTMTRDTSTSTVYMELSSLRSEDT | |
| | AVYYCARGKWWLDYWGKGTLVTVSS | |
| 68 | QSVLTQPPSVSAAPGQKVTISCSGGSSNIGNSYVSWYQQLPGTAPKL | MEDI-1-VL |
| | LIYDNNKRPSGIPDRFSGSKSGTSATLAITGLQTGDEADYYCGTWDT | |
| | SLSANYVFGTGTKLTVL | |
| 69 | QVQLVQSGAEVKKPGASVKVSCKASGYAFTSYYMHWARQAPGQG | MEDI-2-VH |
| | LEWMGIINPSGGSTSYAQKFQGRVTMTRDTSTSTVYMELSSLRSEDT | |
| | AVYYCARGKWWLYNWGKGTLVTVSS | |
| 70 | QSVLTQPPSVSAAPGQKVTISCSGGSSNIGNSYVSWYQQLPGTAPKL | MEDI-2-VL |
| | LIYDNNKRPSGIPDRFSGSKSGTSATLAITGLQTGDEADYYCGTWDT | |
| | SQPPNPLFGTGTKLTVL | |
| 71 | QVQLVQSGAEVKKPGASVKVSCKASGYAFTSYYMHWARQAPGQG | MEDI-3-VH |
| | LEWMGIINPSGGSTSYAQKFQGRVTMTRDTSTSTVYMELSSLRSEDT | |
| | AVYYCARGKLLKNPWGKGTLVTVSS | |
| 72 | QSVLTQPPSVSAAPGQKVTISCSGGSSNIGNSYVSWYQQLPGTAPKL | MEDI-3-VL |
| | LIYDNNKRPSGIPDRFSGSKSGTSATLAITGLQTGDEADYYCGTWFG | |
| | TPASNYVFGTGTKLTVL | |
| 73 | QVQLVQSGAEVKKPGASVKVSCKASGYAFTSYYMHWARQAPGQG | MEDI-4-VH |
| | LEWMGIINPSGGSTSYAQKFQGRVTMTRDTSTSTVYMELSSLRSEDT | |
| | AVYYCARGKWWLYNWGKGTLVTVSS | |
| 74 | QSVLTQPPSVSAAPGQKVTISCSGGSSNIGNSYVSWYQQLPGTAPKL | MEDI-4-VL |
| | LIYDNNKRPSGIPDRFSGSKSGTSATLAITGLQTGDEADYYCGTWDT | |
| | SSPPQPIFGTGTKLTVL | |
| 75 | QVQLVQSGAEVKKPGASVKVSCKASGYAFTSYYMHWARQAPGQG | MEDI-5-VH |
| | LEWMGIINPSGGSTSYAQKFQGRVTMTRDTSTSTVYMELSSLRSEDT | |
| | AVYYCARGKWWLYDWGKGTLVTVSS | |
| 76 | QSVLTQPPSVSAAPGQKVTISCSGGSSNIGNSYVSWYQQLPGTAPKL | MEDI-5-VL |
| | LIYDNNKRPSGIPDRFSGSKSGTSATLAITGLQTGDEADYYCGTWDT | |
| | SSPPQPIFGTGTKLTVL | |
| 77 | QVQLVQSGAEVKKPGASVKVSCKASGYAFTSYYMHWARQAPGQG | MEDI-6-VH |
| | LEWMGIINPSGGSTSYAQKFQGRVTMTRDTSTSTVYMELSSLRSEDT | |
| | AVYYCARGKYWMYDWGKGTLVTVSS | |
| 78 | QSVLTQPPSVSAAPGQKVTISCSGGSSNIGNSYVSWYQQLPGTAPKL | MEDI-6-VL |
| | LIYDNNKRPSGIPDRFSGSKSGTSATLAITGLQTGDEADYYCGTWDT | |
| | STTYHPIFGTGTKLTVL | |
| 79 | QVQLVQSGAEVKKPGASVKVSCKASGYAFTSYYMHWARQAPGQG | MEDI-7-VH |
| | LEWMGIINPSGGSTSYAQKFQGRVTMTRDTSTSTVYMELSSLRSEDT | |
| | AVYYCARGKWWWQYWGKGTLVTVSS | |
| 80 | QSVLTQPPSVSAAPGQKVTISCSGGSSNIGNSYVSWYQQLPGTAPKL | MEDI-7-VL |
| | LIYDNNKRPSGIPDRFSGSKSGTSATLAITGLQTGDEADYYCGTWDT | |
| | SSPPQPIFGTGTKLTVL | |
| 81 | QVQLVQSGAEVKKPGASVKVSCKASGYAFTSYYMHWARQAPGQG | MEDI-8-VH |
| | LEWMGIINPSGGSTSYAQKFQGRVTMTRDTSTSTVYMELSSLRSEDT | |
| | AVYYCARGKWWWQYWGKGTLVTVSS | |
| 82 | QSVLTQPPSVSAAPGQKVTISCSGGSSNIGNSYVSWYQQLPGTAPKL | MEDI-8-VL |
| | LIYDNNKRPSGIPDRFSGSKSGTSATLAITGLQTGDEADYYCGTWDT | |
| | STTYHPIFGTGTKLTVL | |
| 83 | QVQLVQSGAEVKKPGASVKVSCKASGYAFTSYYMHWARQAPGQG | MEDI-9-VH |
| | LEWMGIINPSGGSTSYAQKFQGRVTMTRDTSTSTVYMELSSLRSEDT | |
| | AVYYCARGKWWLYNWGKGTLVTVSS | |
| 84 | QSVLTQPPSVSAAPGQKVTISCSGGSSNIGNSYVSWYQQLPGTAPKL | MEDI-9-VL |
| | LIYDNNKRPSGIPDRFSGSKSGTSATLAITGLQTGDEADYYCGTWDT | |
| | STTMYPLFGTGTKLTVL | |
| 85 | QVQLVQSGAEVKKPGASVKVSCKASGYAFTSYYMHWARQAPGQG | MEDI-10-VH |
| | LEWMGIINPSGGSTSYAQKFQGRVTMTRDTSTSTVYMELSSLRSEDT | |
| | AVYYCARGKWWLYDWGKGTLVTVSS | |
| 86 | QSVLTQPPSVSAAPGQKVTISCSGGSSNIGNSYVSWYQQLPGTAPKL | MEDI-10-VL |
| | LIYDNNKRPSGIPDRFSGSKSGTSATLAITGLQTGDEADYYCGTWDT | |
| | STVLTPIFGTGTKLTVL | |
| 87 | QVQLVQSGAEVKKPGASVKVSCKASGYAFTSYYMHWARQAPGQG | MEDI-11-VH |
| | LEWMGIINPSGGSTSYAQKFQGRVTMTRDTSTSTVYMELSSLRSEDT | |
| | AVYYCARGKWWFYDWGKGTLVTVSS | |
| 88 | QSVLTQPPSVSAAPGQKVTISCSGGSSNIGNSYVSWYQQLPGTAPKL | MEDI-11-VL |
| | LIYDNNKRPSGIPDRFSGSKSGTSATLAITGLQTGDEADYYCGTWDT | |
| | SPSMIPLFGTGTKLTVL | |
| 89 | QVQLVQSGAEVKKPGASVKVSCKASGYAFTSYYMHWARQAPGQG | MEDI-12-VH |
| | LEWMGIINPSGGSTSYAQKFQGRVTMTRDTSTSTVYMELSSLRSEDT | |
| | AVYYCARGKWWFYDWGKGTLVTVSS | |
| 90 | QSVLTQPPSVSAAPGQKVTISCSGGSSNIGNSYVSWYQQLPGTAPKL | MEDI-12-VL |
| | LIYDNNKRPSGIPDRFSGSKSGTSATLAITGLQTGDEADYYCGTWDT | |
| | STTMYPLFGTGTKLTVL | |
| 91 | QVQLVQSGAEVKKPGASVKVSCKASGYAFTSYYMHWARQAPGQG | MEDI-13-VH |
| | LEWMGIINPSGGSTSYAQKFQGRVTMTRDTSTSTVYMELSSLRSEDT | |
| | AVYYCARGKWWLYDWGKGTLVTVSS | |
| 92 | QSVLTQPPSVSAAPGQKVTISCSGGSSNIGNSYVSWYQQLPGTAPKL | MEDI-13-VL |
| | LIYDNNKRPSGIPDRFSGSKSGTSATLAITGLQTGDEADYYCGTWDT | |
| | STTLQPLFGTGTKLTVL | |
| 93 | QVQLVQSGAEVKKPGASVKVSCKASGYAFTSYYMHWARQAPGQG | MEDI-14-VH |
| | LEWMGIINPSGGSTSYAQKFQGRVTMTRDTSTSTVYMELSSLRSEDT | |
| | AVYYCARGKWWLYNWGKGTLVTVSS | |
| 94 | QSVLTQPPSVSAAPGQKVTISCSGGSSNIGNSYVSWYQQLPGTAPKL | MEDI-14-VL |
| | LIYDNNKRPSGIPDRFSGSKSGTSATLAITGLQTGDEADYYCGTWDT | |
| | SPPTKPLFGTGTKLTVL | |
| 95 | QVQLVQSGAEVKKPGASVKVSCKASGYAFTSYYMHWARQAPGQG | MEDI-15-VH |
| | LEWMGIINPSGGSTSYAQKFQGRVTMTRDTSTSTVYMELSSLRSEDT | |
| | AVYYCARGKWWLYNWGKGTLVTVSS | |
| 96 | QSVLTQPPSVSAAPGQKVTISCSGGSSNIGNSYVSWYQQLPGTAPKL | MEDI-15-VL |
| | LIYDNNKRPSGIPDRFSGSKSGTSATLAITGLQTGDEADYYCGTWDT | |
| | STHRHPLFGTGTKLTVL | |
| 97 | QVQLVQSGAEVKKPGASVKVSCKASGYAFTSYYMHWARQAPGQG | MEDI-16-VH |
| | LEWMGIINPSGGSTSYAQKFQGRVTMTRDTSTSTVYMELSSLRSEDT | |
| | AVYYCARGKWWLYNWGKGTLVTVSS | |
| 98 | QSVLTQPPSVSAAPGQKVTISCSGGSSNIGNSYVSWYQQLPGTAPKL | MEDI-16-VL |
| | LIYDNNKRPSGIPDRFSGSKSGTSATLAITGLQTGDEADYYCGTWDT | |
| | STTYHPIFGTGTKLTVL | |
| 99 | QVQLVQSGAEVKKPGASVKVSCKASGYAFTSYYMHWARQAPGQG | MEDI-17-VH |
| | LEWMGIINPSGGSTSYAQKFQGRVTMTRDTSTSTVYMELSSLRSEDT | |
| | AVYYCARGKWWWQHWGKGTLVTVSS | |
| 100 | QSVLTQPPSVSAAPGQKVTISCSGGSSNIGNSYVSWYQQLPGTAPKL | MEDI-17-VL |
| | LIYDNNKRPSGIPDRFSGSKSGTSATLAITGLQTGDEADYYCGTWDT | |
| | SPVDRPIFGTGTKLTVL | |
| 101 | QVQLVQSGAEVKKPGASVKVSCKASGYAFTSYYMHWARQAPGQG | MEDI-18-VH |
| | LEWMGIINPSGGSTSYAQKFQGRVTMTRDTSTSTVYMELSSLRSEDT | |
| | AVYYCARGKWWWQHWGKGTLVTVSS | |
| 102 | QSVLTQPPSVSAAPGQKVTISCSGGSSNIGNSYVSWYQQLPGTAPKL | MEDI-18-VL |
| | LIYDNNKRPSGIPDRFSGSKSGTSATLAITGLQTGDEADYYCGTWDT | |
| | STTPMPVFGTGTKLTVL | |
| 103 | QVQLVQSGAEVKKPGASVKVSCKASGYAFTSYYMHWARQAPGQG | MEDI-19-VH |
| | LEWMGIINPSGGSTSYAQKFQGRVTMTRDTSTSTVYMELSSLRSEDT | |
| | AVYYCARGKWWWQHWGKGTLVTVSS | |
| 104 | QSVLTQPPSVSAAPGQKVTISCSGGSSNIGNSYVSWYQQLPGTAPKL | MEDI-19-VL |
| | LIYDNNKRPSGIPDRFSGSKSGTSATLAITGLQTGDEADYYCGTWDT | |
| | STTYHPIFGTGTKLTVL | |
| 105 | QVQLVQSGAEVKKPGASVKVSCKASGYAFTSYYMHWARQAPGQG | MEDI-20-VH |
| | LEWMGIINPSGGSTSYAQKFQGRVTMTRDTSTSTVYMELSSLRSEDT | |
| | AVYYCARGKYWMYDWGKGTLVTVSS | |
| 106 | QSVLTQPPSVSAAPGQKVTISCSGGSSNIGNSYVSWYQQLPGTAPKL | MEDI-20-VL |
| | LIYDNNKRPSGIPDRFSGSKSGTSATLAITGLQTGDEADYYCGTWDT | |
| | STVWEWPFGTGTKLTVL | |
| 107 | QVQLVQSGAEVKKPGASVKVSCKASGYAFTSYYMHWARQAPGQG | MEDI-21-VH |
| | LEWMGIINPSGGSASYAQKFQGRVTMTRDTSTSTVYMELSSLRSEDT | |
| | AVYYCARGKYWMYDWGKGTLVTVSS | |
| 108 | QSVLTQPPSVSAAPGQKVTISCSGGSSNIGNSYVSWYQQLPGTAPKL | MEDI-21-VL |
| | LIYDNNKRPSGIPDRFSGSKSGTSATLAITGLQTGDEAVYFCGTWDTS | |
| | TVWEWPFGTGTKLTVL | |
| 109 | QVQLVQSGAEVKKPGASVKVSCKASGYAFTSYYMHWARQAPGQG | MEDI-22-VH |
| | LEWMGIINPSGGSTSYAQKFQGRVTMTRDTSTSTVYMELSSLRSEDT | |
| | AVYYCARGKYWMYDWGKGTLVTVSS | |
| 110 | QPVLTQPPSVSAAPGQKVTISCSGGSSNIGNSYVSWYQQLPGTAPKL | MEDI-22-VL |
| | LIYDNNKRPSGIPDRFSGSKSGTSATLAITGLQTGDEADYFCGTWDTS | |
| | TVWEWPFGTGTKLTVL | |
| 111 | QVQLVQSGAEVRKPGASVKVSCKASGYAFTSYYMHWARQAPGQG | MEDI-23-VH |
| | LEWMGIINPSGGSTSYAQKFQGRVTMTRDTSTSTVYMELSSLRSEDT | |
| | AVYYCARGKYWMYDWGKGTLVTVSS | |
| 112 | QSVLTQPPSVSAAPGQKVTISCSGGSSNIGNNYVSWYQQLPGTAPKL | MEDI-23-VL |
| | LIYDNNKRPPGIPDRFSGSKSGTSATLAITGLQTGDEADYYCGTWDT | |
| | STVWEWPFGTGTKLTVL | |
| 113 | QVQLVQSGAEVKKPGASVKVSCKASGYAFTSYYMHWARQAPGQG | MEDI-24-VH |
| | LEWMGIINPRGGSTSYAQKFQGRVTMTRDTSTSTVYMELSSLRSEDT | |
| | AVYYCARGKYWMYDWGKGTLVTVSS | |
| 114 | QSVLTQPPSVSAAPGQKVTISCSGGSSNIGNSYVSWYQQLPGTAPKL | MEDI-24-VL |
| | LIYDNNKRPSGIPDRFSGSKSGTSATLAITGLQTGDEADYFCGTWDTS | |
| | TVWEWPFGTGTKLTVL | |
| 115 | QVQLVQSGAEVKKPGASVKVSCKASGYAFTSYYMHWARQAPGQG | MEDI-25-VH |
| | LEWMGIINPRGGSASYAQKFQGRVSMTRDTSTSTVYMELSSLRSEDT | |
| | AVYYCARGKYWMYDWGKGTLVTVSS | |
| 116 | QSVLTQPPSVSAAPGQKVTISCSGGSSNIGNSYVSWYQQLPGTAPKL | MEDI-25-VL |
| | LIYDNNKRPSGIPDRFSGSKSGTTATLAITGLQTGDEADYYCGTWVT | |
| | STVWEWPFGTGTKLTVL | |
| 117 | QVQLVQSGAEVKKPGASVKVSCKASGYAFTSYYMHWARQAPGQG | MEDI-26-VH |
| | LEWMGIINPSGGSTSYAQKFQGRVTMTRDTSTSTVYMELSSLRSEDT | |
| | AVYYCARGKYWMYDWGKGTLVTVSS | |
| 118 | QSVLTQPPSVSAAPGQKVTISCSGGSSNIGNSYVSWYQQLPGTAPKL | MEDI-26-VL |
| | LIYDNNKRPSGIPDRFSGSKSGTSATLAITGLQTGDEADYFCGTWDTS | |
| | TVWEWPFGTGTKLTVL | |
| 119 | QVQLVQSGAEVRKPGASVKVSCKASGYAFTSYYMHWARQAPGQG | MEDI-27-VH |
| | LEWMGIINPSGGSTSYAQKFQGRVTMTRDTSTSTVYMELSSLRPEDT | |
| | AVYYCARGKYWMYDWGKGTQVTVSS | |
| 120 | QSVLTQPPLVSAAPGQKVTISCSGGSSNIGNSYVSWYQRLPGTAPKL | MEDI-27-VL |
| | LIYDNNKRPSGIPDRFSGSKSGTSATLAITGLQTGDEADYYCGTWDT | |
| | STVWEWPFGTGTKLTVL | |
| 121 | QVQLVQSGAEVKKPGASVKVSCKASGYAFTSYYMHWARQAPGQG | MEDI-28-VH |
| | LEWMGIINPSGGSTSYAQKFQGRVTMTRDTSTSTVYMELSSLRSEDT | |
| | AVYYCARGKYWMYDWGNGTLVTVSS | |
| 122 | LPVLTQPPSVSAAPGQKVTISCSGGSSSIGNSYVSWYQQLPGAAPKLL | MEDI-28-VL |
| | IYDNNKRPSGIPDRFSGFRSGTSATLAITGLQTGDEADYYCGTWDTSP | |
| | VWEWPFGTGTKLTVL | |
| 123 | QVQLVQSGAEVKKPGASVKVSCKASGYAFTSYYMHWARQAPGQG | MEDI-29-VH |
| | LEWMGIINPSGGSTSYAQKFQGRVTMTRDTSTSTVYMELSSLRSEDT | |
| | AVYYCARGKYWMYDWGKGTRVTVSS | |
| 124 | QSVLTQPPSVSAAPGQKVTISCSGGSSNIGNSYVSWYQQLPGTAPKL | MEDI-29-VL |
| | LIYDNNKRPSGIPDRFSGSKSGTSATLAITGLQTGDEADYYCGTWDT | |
| | SPVWEWPFGTGTKLTVL | |
| 125 | QVQLVQSGAEVKKPGASVKVSCKASGYAFTSYYMHWARQAPGQG | MEDI-30-VH |
| | LEWMGIINPSGGSTSYAQKFQGRVTMTRDTSTSTVYMELSSLRSEDT | |
| | AVYYCARGKYWMYDWGKGTLVTVSS | |
| 126 | QSVLTQPPSVSAAPGQKVTISCSGGSSNIGNSYVSWYQRLPGAAPKL | MEDI-30-VL |
| | LIYDNNKRPSGIPDRFSGSKSGTSATLAITGLQTGDEADYYCGTWDT | |
| | STVWEWPFGTGTKLTVL | |
| 127 | QVQLVQSGAEVKKPGASVKVSCKASGYAFTSYYMHWARQAPGQG | MEDI-31-VH |
| | LEWMGIINPSGGSTSYAQKFQGRVTMTRDTSTSTVYMELSSLRSEDT | |
| | AVYYCARGKYWMYDWGKGTLVTVSS | |
| 128 | QSVLTQPPSVSAAPGQKVTISCSGGSSSIGNSYVSWYQQLPGTAPKLL | MEDI-31-VL |
| | IYDNNKRPSGIPDRFSGSKSGTSATLAITGLQTGDEADYYCGTWATS | |
| | PVWEWPFGTGTKLTVL | |
| 129 | QVQLVQSGAEVKKPGASVKVSCKASGYAFTSYYMHWARQAPGQG | MEDI-32-VH |
| | LEWMGIINPSGGSTSYAQKFQGRVTMTRDTSTSTVYMELSSLRSEDT | |
| | AVYYCARGKYWMYDWGKGTLVTVSS | |
| 130 | QSVLTQPPSVSAAPGQKVTISCSGGSSNIGNSYVSWYQQLPGTAPKL | MEDI-32-VL |
| | LIYDNNKRPSGIPDRFSGSKSGTSATLAITGLQTGDEADYFCGTWDTS | |
| | TAWEWPFGTGTKLTVL | |
| 131 | QVQLVQSGAEEKKPGASVKVSCKASGYAFTSYYMHWARQAPGQG | MEDI-33-VH |
| | LEWMGIINPSGGSTSYAQKFQGRVTMTRDTSTSTVYMELSSLRSEDT | |
| | AVYYCARGKYWMYDWGKGTLVTVSS | |
| 132 | QSALTQPPSVSAAPGQKVTISCSGGSSNIGNSYVSWYQQLPGTAPKL | MEDI-33-VL |
| | LIYDNNKRPSGIPDRFSGSKSGTSATLAITGLQTGDEADYFCGTWDTS | |
| | TVWEWPFGTGTKLTVL | |
| 133 | QVQLVQSGAEVKKPGASVKVSCKASGYAFTSYYMHWARQAPGQG | MEDI-34-VH |
| | LEWMGIINPSGGSTSYAQKFQGRVSMTRDTSTSTVYMELSSLRSEDT | |
| | AVYYCARGKYWMYDWGKGTLVTVSS | |
| 134 | QSVLTQPPSVSAAPGQKVTISCSGGSSNIGNSYVSWYQQLPGTAPKL | MEDI-34-VL |
| | LIYDNNKRPSGIPDRFSGSKSGTSATLAITGLQTGDEADYFCGTWDTS | |
| | TVWEWPFGTGTKLTVL | |
| 135 | QVQLVQSGAEVKKPGASVKVSCKASGYAFTSYYMHWARQAPGQG | MEDI-35-VH |
| | LEWMGIINPSGGSTSYAQKFQGRVTMTRDTSTSTVYMELSSLRSEDT | |
| | AVYYCARGKYWMYDWGKGTLVTVSS | |
| 136 | QSVLTQPPSVSAAPGQKVTISCSGGSSNIGNSYVSWYQQLPGTAPKL | MEDI-35-VL |
| | LIYDNNKRPSGIPDRFSGSKSGTSATLAITGLQTGDEADYYCGTWDT | |
| | SPVWEWPFGTGTKLTVL | |
| 137 | QVQLVQSGAEVKKPGASVKVSCKASGYAFTSYYMHWARQAPGQG | MEDI-36-VH |
| | LEWMGIINPSGGSASYAQKFQGRVTMTRDTSTSTVYMELSSLRSEDT | |
| | AVYYCARGKYWMYDWGKGTLVTVSS | |
| 138 | QSVLTQPPSVSAAPGQKVTISCSGGSSNIGNSYVSWYQQLPGTAPKL | MEDI-36-VL |
| | LIYDNNKRPSGIPDRFSGSKSGTSATLAITGLQTGDEADYYCGTWDS | |
| | STVWEWPFGTGTKLTVL | |
| 139 | QVQLVQSGAEVKKPGASVKVSCKASGYAFTSYYMHWARQAPGQG | MEDI-37-VH |
| | LEWMGIINPRGGSTSYAQKFQGRVAMTRDTSTSTVYMELSSLRPEDT | |
| | AVYYCARGKYWMYDWGKGTLVTVSS | |
| 140 | QSVLTQPPSVSAAPGQKVTISCSGGGSSIGNSYVSWYQQLPGTAPKL | MEDI-37-VL |
| | LIYDNNKRPSGVPDRFSGSKSGTSATLAITGLQTGDEADYYCGTWDT | |
| | SPVWEWPFGTGTKLTVL | |
| 141 | QVQLVQSGAEVKKPGASVKVSCKASGYAFTSYYMHWARQAPGQG | MEDI-38-VH |
| | LEWMGIINPSGGSASYAQKFQGRVTMTRDTSTSTVYMELSSLRSEDT | |
| | AVYYCARGKYWMYDWGKGTLVTVSS | |
| 142 | QSVLTQPPSVSAAPGQKVTISCSGGSSNIGNSYVSWYQQLPGTAPKL | MEDI-38-VL |
| | LIYDNNKRPSGIPDRFSGSKSGTSATLAITGLQTGDEADYFCGTWDTS | |
| | TVWEWPFGTGTKLTVL | |
| 143 | QVQLVQSGAEVKKPGASVKVSCKASGYAFTSYYMHWARQAPGQG | MEDI-39-VH |
| | LEWMGIINPRGGSTSYAQKFQGRVTMTRDTSTSTVYMELSSLRSEDT | |
| | AVYYCARGKYWMYDWGKGTLVTVSS | |
| 144 | QSVLTQPPSVSAAPGQKVTISCSGGSSNIGNSYVSWYQQLPGTAPKL | MEDI-39-VL |
| | LIYDNNKRPSGIPDRFSGSKSGTSATLAITGLQTGDEADYYCGTWDT | |
| | STAWEWPFGTGTKLTVL | |
| 145 | QVQLVQSGAEVKKPGASVKVSCKASGYAFTSYYMHWARQAPGQG | MEDI-40-VH |
| | LEWMGIINPSGGSTSYAQKFQGRVTMTRDTSTSTVYMELSSLRSEDT | |
| | AVYYCARGKYWMYDWGKGTLVTVSS | |
| 146 | QSVLTQPPSVSAAPGQKVTISCSGGSSNIGNSYVSWYQQLPGTAPKL | MEDI-40-VL |
| | LIYDNNKRPSGIPDRFSGSKSGTSATLAITGLQTGDEADYYCGTWDS | |
| | STVWEWPFGTGTKLTVL | |
| 147 | QVQLVQSGAEVRKPGASVKVSCKASGYAFTSYYMHWARQAPGQG | MEDI-41-VH |
| | LEWMGIINPSGGSTSYAQKFQGRVTMTRDTSTSTVYMELSSLRPEDT | |
| | AVYYCARGKYWMYDWGKGTLVTVSG | |
| 148 | QSVLTQPPSVSAAPGQKVTISCSGGSTNIGNSYVSWYQRLPGTAPKL | MEDI-41-VL |
| | LIYDNNKRPPGIPDRFSGSKSGTSATLAITGLQTGDEADYYCGTWDT | |
| | STVWEWPFGTGTKLTVL | |
| 149 | QVQLVQSGAEVKKPGASVKVSCKASGYAFTSYYMHWARQAPGQG | MEDI-42-VH |
| | LEWVGIINPSGGSTSYAQKFQGRVTMTRDTSTSTVYMELSSLRSGDT | |
| | AVYYCARGKYWMYDWGKGTLVTVSS | |
| 150 | QAVLTQPPSVSAAPGQKVTISCSGGSSNIGNSYVSWYQRLPGAAPKL | MEDI-42-VL |
| | LIYDNNKRPSGIPDRFSGSKSGTSATLAITGLQTGDEADYYCGTWDT | |
| | STGWEWPFGTGTKLTVL | |
| 151 | QVQLVQSGAEVKKPGASVKVSCKASGYAFTSYYMHWVRQAPGQG | MEDI-37GL-VH |
| | LEWMGIINPRGGSTSYAQKFQGRVTMTRDTSTSTVYMELSSLRSEDT | |
| | AVYYCARGKYWMYDWGKGTLVTVSS | |
| 152 | QSVLTQPPSVSAAPGQKVTISCSGGGSSIGNSYVSWYQQLPGTAPKL | MEDI-37GL-VL |
| | LIYDNNKRPSGIPDRFSGSKSGTSATLGITGLQTGDEADYYCGTWDT | |
| | SPVWEWPFGTGTKLTVL | |
| 153 | EVQLLESGGGLVQPGGSLRLSCAVSGFTFSNYAMSWVRQAPGKGLE | AJOU-1-VH |
| | WVSAISSGGGNIYYADSVKGRFTISRDNSKNTLYLQMNSLRAEDTA | |
| | VYYCAKLRRYFDYWGQGTLVTVSS | |
| 154 | EVQLLESGGGLVQPGGSLRLSCAASGFTFSDYAMSWVRQAPGKGLE | AJOU-2-VH |
| | WVSAISSGGSSIYYADSVKGRFTISRDNSKNTLHLQMNSLRAEDTAV | |
| | YYCARGPQRSATAVFDYWGQGTLVTVSS | |
| 155 | EVQLLESGGGLVQPGGSLRLSCAASGFTFSNYAMSWVRQAPGKGLE | AJOU-3-VH |
| | WVSWISPNSGNIYYADSVKGRFTISRDNSKNTLYLQMNSLRAEDTA | |
| | VYYCARRPLSAAWSHSSYYNAMDVWGQGTLVTVSS | |
| 156 | EVQLLESGGGLVQPGGSLRLSCAASGFTFSGYAMSWVRQAPGKGLE | AJOU-4-VH |
| | WVSLISHSGSNTYYADSVKGRFTISRDNSKNTLYLQMNSLRAEDTA | |
| | VYYCARPHRAFDYWGQGTLVTVSS | |
| 157 | EVQLLESGGGLVQPGGSLRLSCAASGFTFSNYAMSWVRQAPGKGLE | AJOU-5-VH |
| | WVSGISHGSGSIYYADSVKGRFTISRDNSKNTLYLQMNSLRAEDTAV | |
| | YYCARPHRAFDYWGQGTLVTVSS | |
| 158 | EVQLLESGGGLVQPGGSLRLSCAASGFTFSNYAMSWVRQAPGKGLE | AJOU-6-VH |
| | WVSGISHGNGSIYYADSVKGRFTISRDNSKNTLYLQMNSLRAEDTA | |
| | VYYCAKTGRHFDYWGQGTLVTVSS | |
| 159 | EVQLLESGGGLVQPGGSLRLSCAASGFTFSNYAMSWVRQAPGKGLE | AJOU-7-VH |
| | WVSSISPSGSSIYYADSVKGRFTISRDNSKNTLYLQMNSLRAEDTAV | |
| | YYCARSYRAFDYWGQGTLVTVSS | |
| 160 | EVQLLESGGGLVQPGGSLRLSCAASGFTFSNYAMSWVRQAPGKGLE | AJOU-8-VH |
| | WVSAISPSGGSIYYADSVKGRFTISRDNSKNTLYLQMNSLRAEDTAV | |
| | YYCARAKRAFDYWGQGTLVTVSS | |
| 161 | EVQLLESGGGLVQPGGSLRLSCAASGFTFSNYAMSWVRQAPGKGLE | AJOU-9-VH |
| | WVSAISPGSGSTYYADSVKGRFTISRDNSKNTLYLQMNSLRAEDTAV | |
| | YYCAKFRRHFDYWGQGTLVTVSS | |
| 162 | EVQLLESGGGLVQPGGSLRLSCAASGFTFSNYAMSWVRQAPGKGLE | AJOU-10-VH |
| | WVSAISSGGGNIYYADSVKGRFTISRDNSKNTLYLQMNSLRAEDTA | |
| | VYYCARVHRAFDYWGQGTLVTVSS | |
| 163 | EVQLLESGGGLVQPGGSLRLSCAASGFTFSNYAMSWVRQAPGKGLE | AJOU-69-VH |
| | WVSAITSSGRSIYYADSVKGRFTISRDNSKNTLYLQMNSLRAEDTAV | |
| | YYCARVHRAFDYWGQGTLVTVSS | |
| 164 | EVQLLESGGGLVQPGGSLRLSCAASGFTFSNYAMSWVRQAPGKGLE | AJOU-70-VH |
| | WVSAITSSGANIYYADSVKGRFTISRDNSKNTLYLQMNSLRAEDTAV | |
| | YYCARVHRAFDYWGQGTLVTVSS | |
| 165 | EVQLLESGGGLVQPGGSLRLSCAASGFTFSNYAMSWVRQAPGKGLE | AJOU-71-VH |
| | WVSAITSSGGNIYYADSVKGRFTISRDNSKNTLYLQMNSLRAEDTAV | |
| | YYCARVHRAFDYWGQGTLVTVSS | |
| 166 | EVQLLESGGGLVQPGGSLRLSCAASGFTFSNYAMSWVRQAPGKGLE | AJOU-72-VH |
| | WVSAITAGGGSIYYADSVKGRFTISRDNSKNTLYLQMNSLRAEDTA | |
| | VYYCARVHRAFDYWGQGTLVTVSS | |
| 167 | EVQLLESGGGLVQPGGSLRLSCAASGFTFSRHAMAWVRQAPGKGLE | AJOU-83-VH |
| | WVSAITSSGRSIYYADSVKGRFTISRDNSKNTLYLQMNSLRAEDTAV | |
| | YYCARVHRAFDYWGQGTLVTVSS | |
| 168 | QSVLTQPPSASGTPGQRVTISCSGSSSNIGNNYVNWYQQLPGTAPKL | AJOU-33-VL |
| | LIYDNSHRPSGVPDRFSGSKSGTSASLAISGLRSEDEADYYCGTWDA | |
| | SLSAYVFGGGTKLTVL | |
| 169 | QSVLTQPPSASGTPGQRVTISCSGSSSNIGNNNVSWYQQLPGTAPKLL | AJOU-34-VL |
| | IYANSKRPSGVPDRFSGSKSGTSASLAISGLRSEDEADYYCGSWDDS | |
| | LSAYVFGGGTKLTVL | |
| 170 | QSVLTQPPSAPGTPGQRVTISCTGSSSNIGSNSVNWYQQLPGTAPKLL | AJOU-35-VL |
| | IYDDSHRPSGVPDRFSGSKSGTSASLAISGLRSEDEADYYCDAWDSS | |
| | LSAYVFGGGTKLTVL | |
| 171 | QSVLTQPPSASGTPGQRVTLSCTGSSSNIGSNYVSWYQQLPGTAPKL | AJOU-36-VL |
| | LIYADSQRPSGVPDRFSGSKSGTSASLAISGLRSEDEADYYCGTWDD | |
| | SLSGYVFGGGTKLTVL | |
| 172 | QSVLTQPPSASGTPGQRVTISCSSSSSNIGSNYVSWYQQLPGTAPKLLI | AJOU-37-VL |
| | YSDSHRPSGVPDRFSGSKSGTSASLAISGLRSEDEADYYCGSWDYSL | |
| | SAYVFGGGTKLTVL | |
| 173 | QSVLTQPPSASGTPGQRVTISCTGSSSNIGNNTVSWYQQLPGTAPKLL | AJOU-38-VL |
| | IYDNSHRPSGVPDRFSGSKSGTSASLAISGLQSEDEADYYCGSWDYS | |
| | LSAYVFGGGTKLTVL | |
| 174 | QSVLTQPPSASGTPGQRVTISCTGSSSNIGNNDVNWYQQLPGTAPKL | AJOU-39-VL |
| | LIYYDSQRPSGVPDRFSGSKSGTSASLAISGLRSEDEADYYCATWDA | |
| | SLSAYVFGGGTKLTVL | |
| 175 | QSVLTQPPSASGTPGQRVTISCSGSSSNIGSNAVNWYQQLPGTAPKLL | AJOU-40-VL |
| | IYYDNQRPSGVPDRFSGSKSGTSASLAISGLRSEDEADYYCGTWDDS | |
| | LNGYVFGGGTKLTVL | |
| 176 | QSVLTQPPSASGTPGQRVTISCSGSSSNIGNNAVTWYQQLPGTAPKLL | AJOU-41-VL |
| | IYDDSHRPSGVPDRFSGSKSGTSASLAISGLRSEDEADYYCGSWDYS | |
| | LSAYVFGGGTKLTVL | |
| 177 | QSVLTQPPSASGTPGQRVTISCSGSSSNIGSNTFNWYQQLPGTAPKLLI | AJOU-42-VL |
| | YADSHRPSGVPDRFSGSKSGTSASLAISGLRSEDEADYYCGTWDYSL | |
| | SGYVLGGGTKLTVL | |
| 178 | QSVLTQPPSASGTPGQRVTISCSGSSSNIGSNTFNWYQQLPGTAPKLLI | AJOU-77-VL |
| | YADSHRPSGVPDRFSGSKSGTSASLAISGLRSEDEADYYCGTWDYSL | |
| | SGYVLGGGTKLTVL | |
| 179 | QSVLTQPPSASGTPGQRVTISCSGSSSNIGSNTFNWYQQLPGTAPKLLI | AJOU-78-VL |
| | YADSHRPSGVPDRFSGSKSGTSASLAISGLRSEDEADYYCGTWDYSL | |
| | RGYVLGGGTKLTVL | |
| 180 | QSVLTQPPSASGTPGQRVTISCSGSSSNIGSNTFNWYQQLPGTAPKLLI | AJOU-79-VL |
| | YADSHRPSGVPDRFSGSKSGTSASLAISGLRSEDEADYYCGYWDYSL | |
| | SGYVLGGGTKLTVL | |
| 181 | QSVLTQPPSASGTPGQRVTISCSGSSSNIGSNTFNWYQQLPGTAPKLLI | AJOU-80-VL |
| | YADSHRPSGVPDRFSGSKSGTSASLAISGLRSEDEADYYCGTWDYSL | |
| | SGYVLGGGTKLTVL | |
| 182 | QSVLTQPPSASGTPGQRVTISCSGSSANSRTDGFNWYQQLPGTAPKL | AJOU-86-VL |
| | LIYADSHRPSGVPDRFSGSKSGTSASLAISGLRSEDEADYYCGTWDY | |
| | SLSGYVLGGGTKLTVLG | |
| 183 | QSVLTQPPSASGTPGQRVTISCSGSAQFGSRDNFNWYQQLPGTAPKL | AJOU-87-VL |
| | LIYADSHRPSGVPDRFSGSKSGTSASLAISGLRSEDEADYYCGTWDY | |
| | SLSGYVLGGGTKLTVLG | |
| 184 | QSVLTQPPSASGTPGQRVTISCSGSTKQMHNYQFNWYQQLPGTAPK | AJOU-88-VL |
| | LLIYADSHRPSGVPDRFSGSKSGTSASLAISGLRSEDEADYYCGTWD | |
| | YSLSGYVLGGGTKLTVLG | |
| 185 | QSVLTQPPSASGTPGQRVTISCSGSLLRGENLQFNWYQQLPGTAPKL | AJOU-89-VL |
| | LIYADSHRPSGVPDRFSGSKSGTSASLAISGLRSEDEADYYCGTWDY | |
| | SLSGYVLGGGTKLTVLG | |
| 186 | QSVLTQPPSASGTPGQRVTISCSGSPLFPDSGSFNWYQQLPGTAPKLL | AJOU-90-VL |
| | IYADSHRPSGVPDRFSGSKSGTSASLAISGLRSEDEADYYCGTWDYS | |
| | LSGYVLGGGTKLTVLG | |
| 187 | QSVLTQPPSASGTPGQRVTISCSGSAALDLSPSFNWYQQLPGTAPKLL | AJOU-91-VL |
| | IYADSHRPSGVPDRFSGSKSGTSASLAISGLRSEDEADYYCGTWDYS | |
| | LSGYVLGGGTKLTVLG | |
| 188 | QVQLVQSGAEVKKPGASVKVSCKASGYTFTNYGISWVRQAPGQGL | REGN-VH-3 |
| | EWMGWISVYNGKTNYAQKLQGRVTMTTDTSTTTAYMEMRSLRSD | |
| | DTAVYYCARGSGYDLDYWGQGTLVSVSS | |
| 189 | EVQLVESGGGLVQPGGSLRLSCAASGFTFSSFWMTWVRQAPGKGLE | REGN-VH-19 |
| | WVANIKQDGSEKYYVDSVKGRFTISRDNAKNSLYLQMNSLRAEDT | |
| | AVYYCARDPGRTMVRGGIRYYYGMDVWGQGTTVTVSS | |
| 190 | EVKLAESGGGLVQPGGSLRLSCAASGFTFSSHWMNWVRQAPGKGL | REGN-VH-35 |
| | EWVANIKQDGSDKYYVDSVKGRFTISRDNAKNSLYLQLNSLIAEDT | |
| | AVYYCARDRGVRPPRGAFDIWGQGTMVTVSS | |
| 191 | QVQLVQSGAEVKKPGASVKVSCKASGYTFNSYGISWVRQAPGQGL | REGN-VH-51 |
| | EWMGWIRTYNGNTNYAQKLQGRVTMTTDTSTSTAYMELRSLRSDD | |
| | TAVYYCARDEARIVVAGTTPYYYGMDVWGQGTTVTVSS | |
| 192 | QVQLVESGGGLVQPGGSLRLSCAVSGFTISDHYMSWIRQAPGKGLE | REGN-VH-67 |
| | WISYISSSGSKIYYADSVKGRFTISRDNAKNSLFLQMNSLRAEDTAVY | |
| | YCARTRQLVGDYWGQGTLVTVSS | |
| 193 | EVQLVESGGGLVQPGRSLRLSCAASGFTFDNYAMHWVRQAPGKGL | REGN-VH-83 |
| | EWVSGIRWNSGSIGYADSVKGRFTISRDNAKNSLYLQMNSLRAEDT | |
| | ALYYCAKEGGYSGYRPGPFFDYWGQGTLVTVSS | |
| 194 | QVQLVQSGAEVKKPGASVKVSCKASGYTFTNYGISWVRQAPGQGL | REGN-VH-99 |
| | EWMGWISVYNGHTNYAQKLQGRVTMTTDTSTSTAYMELRSLRSDD | |
| | TAVYYCARGSGYDFDSWGQGTLVTVSS | |
| 195 | QVQLVQSGAEVKKPGASVKVSCKASRYTFTSYDINWVRQATGQGL | REGN-VH-115 |
| | EWMGWMNPNSGNTGYAQKFQGRVTMTRNTSTSTAYMELSSLRSE | |
| | DTAVYYCARVRRFFDYWGQGTLVTVSS | |
| 196 | QVQLVQSGPEVKKPGASVKVSCKASGYTFTNYGISWVRQAPGQGLE | REGN-VH-147 |
| | WMGWISVYNGNINYAQKLQGRVTMTTDTSTSTAYMDLRSLRSDDT | |
| | AVYYCARGSGYDFDYWGQGTLVTVSS | |
| 197 | QVQLVQSGAEVKKPGASVKVSCKDSAYTFNRYGISWVRQAPGQGL | REGN-VH-163 |
| | EWMGWISAYTGNTVYAQKLQGRVTMTTDNSTSTAYMELRSLRSDD | |
| | TAVYYCARDKSIFGVVRGFDYWGQGTLVTVSS | |
| 198 | AIQMTQSPSSLSASVGDRVTITCRASQGIRNALGWYQQKPGKAPKLL | REGN-VL-11 |
| | IYAASSLQSGVPSRFSGSGSGTDFTLTFSSLQPEDFATYYCLQDFNYP | |
| | YTFGQGTKLEIK | |
| 199 | DIQMTQSPSSVSASVGDRVTISCRASQGVSSWLAWYQQKPGNAPKL | REGN-VL-27 |
| | LISAASSIQSGVPSRFSGSGSGTDFTLTISSLQPEDFATYYCQQANSFPL | |
| | TFGGGTKVEIK | |
| 200 | DIQMTQSPSSVSASVGDRVTITCRASQGISSWLAWYQQKPGKAPKLL | REGN-VL-43 |
| | IYAASSFQSGVPSRFSGSGSGTDFTLTISSLQPEDFATYFCQQANSFPL | |
| | TFGGGTTVEIK | |
| 201 | DIQMTQSPSSVSASVGDRVTITCRASQDISIWLAWYQQSPGKAPKLLI | REGN-VL-59 |
| | NVASRLQSGVPSRFSGSGSGTDFTLTINSLQPEDFVTYYCQQANSFPI | |
| | TFGQGTRLATK | |
| 202 | DIQLTQSPSFLSASVGDRVTITCWASQGISSYLAWYQQKPGKAPKLLI | REGN-VL-75 |
| | FAASTLQSGVPSRFSGSGSGTEFTLTISSLOPEDFATYYCQQLNSYPLT | |
| | FGGGTKVEIR | |
| 203 | EIVMTQSPATLSVSPGERATLSCRASQSVNYNLAWYQHKPGQAPRL | REGN-VL-91 |
| | LIYGASTRATGIPARFSGSGSGTEFTLTISSLQSEDFAVYYCQQYNNW | |
| | PLTFGGGTKVEIK | |
| 204 | AIQMTQSSSSLSASVGDRVTITCRASQAIRNALGWYQQKPGKAPKVL | REGN-VL-107 |
| | IYAASSLQSGIPSRFSGSGSGTDFTLTISSLQPEDFATYYCLQDYDYPY | |
| | TFGQGTKLEIK | |
| 205 | DIQLTQSPSFLSASVGDRVTITCWASQGIISYLAWYQQKPGKAPKLLI | REGN-VL-123 |
| | YAASTLHSGVPSRFSGSGSGTEFTLTISSLQPEDFATYYCHQLKSYPIT | |
| | FGQGTRLEIK | |
| 206 | AIQMTQSPSSLSASVGDRVTITCRASQDIRNALGWYQQKPGKAPKLL | REGN-VL-155 |
| | IYAASSLQSGVPSRFSGSASGTDFTLTISSLQPEDFAAYYCLQDYNYP | |
| | YTFGQGTKLEIK | |
| 207 | EIVMTQSPVTLSLSPGERATLPCRASQSVSSSLAWYQQKAGQSPRLLI | REGN-VL-171 |
| | YGASTRATGIPARFSGSGSGTEFTLTISNLQSEDFAVYYCQQYNNWP | |
| | LTFGGGTKVEIK | |

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.), but some experimental errors and deviations should be accounted for. Unless indicated otherwise, parts are parts by weight, molecular weight is average molecular weight, temperature is in degrees Centigrade, and pressure is at or near atmospheric.

### Determination of Subvisible Particles

For the determination of subvisible particles, suitable methods include “Method 1” (Light Obscuration Particle Count Test) and “Method 2” (Microscopic Particle Count Test). Using light obscuration, the FDA requirement for subvisible particulates in parenteral drug product is ≤6,000 particles per container for particles ≥10 micrometers in diameter, and ≤600 particles per container for particles ≥25 micrometers in diameter. Using the microscopic method, the FDA requirement for subvisible particulates in parenteral drug product is ≤3,000 particles per container for particles ≥10 micrometers in diameter, and ≤300 particles per container for particles ≥25 micrometers in diameter. Presently, no specification exists for particles of less than 10 micrometers in diameter, but the FDA has requested that particles of 2 to 10 micrometers be measured.

Particles of greater than 1 micrometer in diameter were measured using HIAC light obscuration and Brightwell micro-flow imaging (MFI). HIAC combines light obscuration with laser light scattering enabling the detection and counting of particles ranging from 500 nm-350 μm in a moving fluid stream. Particles were sized based on voltage response generated in the detector and sorted into pre-determined size ranges based on voltage response.

For HIAC assays, samples from a manufacturing line (GMP lots) containing a monoclonal antibody at 150 mg/mL were pooled to a total volume of 25 mL. For each pooled sample, three readings of five milliliters per sample were made. Laboratory samples of the same 150 mg/mL antibody formulation were also examined by HIAC. Samples from at least three vials (2.5 mL/vial), seven 1-mL syringes (1.14 mL/syringe), or five 2.25-mL syringes (2 mL/syringe) were pooled, and three reading of one milliliter per reading were made. HIAC 9703 and HIAC 8000A instruments (Hach Company, Loveland, Colo.) using the HRLD 400 probe (which reads up to 18,000 cumulative counts per mL) and MC05 probe (which reads up to 10,000 cumulative counts per mL) respectively, were used to make the light obscuration readings.

The MFI method used less material (i.e., 1 mL of formulation, or 1 stability vial or syringe) than HIAC light obscuration and yielded higher particulate numbers than HIAC. Since MFI is microscopy-based, that method was more sensitive to the translucent protein particulates and was able to differentiate silicone oil droplets/air bubbles from protein particulates for prefilled syringe samples. MFI was conducted on a laboratory sample containing 150 mg/mL of a monoclonal antibody (as in the HIAC analyses). For MFI, one reading of one milliliter per reading was made.

### Determination of Polysorbate Degradation

Degradation of polysorbate was examined using one or more of several methods. The first method employed an enzymatic colorimetric assay to quantify non-esterified fatty acids (NEFA). The NEFA-HR (2) kit (Wako Diagnostics, Richmond, Va.) was used to detect fatty acids in formulated drug substance containing polysorbate. Briefly, the samples were combined with ATP and coenzyme A (CoA) in the presence of acyl-CoA synthetase (ACS). Available (free) fatty acids reacted with the CoA to form acyl-CoA. The acyl-CoA product was reacted with oxygen and acyl-CoA oxidase to produce trans-2,3-dehydroacyl-CoA and hydrogen peroxide. Peroxidase catalyzed the reaction of the hydrogen peroxide with 4-aminoantipyrine and 3-methyl-N-ethyl-N-(β-hydroxyethyl)-aniline to form a blue purple pigment (maximum absorbance at 550 nm). The amount of NEFA in the sample is proportional to the amount of pigment. For a detailed description of the NEFA colorimetric assay, see Duncombe, “The Colorimetric Micro-Determination of Non-Esterified Fatty Acids in Plasma,” Clin Chim Acta. 9:122-5 (1964); Itaya and Ui, “Colorimetric Determination of Free Fatty Acids in Biological Fluids,” J. Lipid Res. 6:16-20 (1965); Novak, M., “Colorimetric Ultramicro Method for the Determination of Free Fatty Acids,” J. Lipid Res. 6:431-3 (1965); and Elphick, M. C., “Modified Colorimetric Ultramicro Method for Estimating NEFA in Serum,” J. Clin. Pathol. 21 (5): 567-70 (1968).

The test sample containing the protein of interest (and putative host cell protein contaminant) was applied to a 10 kDa molecular weight cut-off filter. The retentate was recovered in 10 mM histidine (pH 6.0) at greater than 100 g/L protein and spiked with polysorbate to give a test sample of 100 g/L protein, 0.8% (w/v) polysorbate, 10 mM histidine, pH 6.0 (tᵢₙᵢₜᵢₐₗ). The test sample was subjected to 45° C. for 44 hours (t_{final}). Some samples were spiked with oleic acid to evaluate the recovery efficiency of NEFA in the samples. Percent polysorbate degradation was calculated as follows:

$$\frac{( [ NEFA ] {t}_{final} - [ NEFA ] {t}_{initial} )}{[ polysorbate ] - [ NEFA ] {t}_{final}} \times 100 \%$$

The second method for determining polysorbate degradation was based on mass spectrometry. Using LC-MS analysis, this assay allowed the measurement and comparison of the initial percentage of esters and remaining percentage of esters in polysorbates after incubation at 45° C. at different time points.

Briefly, 15 mg of antibody sample (on the order of 5-10 mg/mL, or 7 mg/mL±1.5 mg/mL) was applied to an ultra-filter (Amicon Ultra 50K, Millipore, Billerica, Mass.) and centrifuged at 14,000×g for 15 minutes or until the remaining volume was slightly below the 100 μL marking on the device. 1 μL of 10% polysorbate was added into the spin filter with the concentrated protein followed with vortexing. The sample was recovered by inverted centrifugation for 5 minutes at 1000 g to recover the full volume in the collection tube.

The recovered volume was measured and the concentration of polysorbate calculated. 1 μL of each recovered sample was and diluted 100-fold in a separate tube, and the protein concentration measured with Nanodrop 1000 (Thermo Fisher Scientific, Inc., Wilmington, Del.). The samples were then diluted in histidine buffer (10 mM, pH 6.0) and polysorbate stock to achieve 150 mg/mL protein concentration and 0.2% (w/w) polysorbate concentration.

Time zero (TO) sample (2 μL) was reserved from each sample and stored at −80° C. until used. Samples to be tested were sealed under argon and incubated at 45° C. to induce degradation, and removed for testing at the prescribed time points. 2 μL was taken from each of the samples at each time point and diluted with water to 100 μL. Each diluted time point sample was stored at −80° C. storage. After collection of each time point, the head space of the sample tube was filled with argon gas, the sample container resealed, and the sample returned to the incubator to resume incubation.

The time point samples were analyzed using an anion exchange column (Oasis MAX column, 30 μm, 2.1 mm×20 mm; Waters Corporation, Milford, Mass.) followed at t=5 minutes with reverse phase chromatography (ACQUITY UPLC® BEH 130 C4 column, 1.7 μm, 2.1 mm×50 mm; Waters Corporation, Milford, Mass.). The reverse phase output was connected to a mass spectrometer (Thermo Q-Exactive mass spectrometer; Thermo Fisher Scientific, Inc., Wilmington, Del.). The chromatographic conditions are described in Table 2.

The system was equilibrated with 99% mobile phase A (0.1% formic acid in water) at a flow rate of 0.1 mL/minute for 40 minutes prior to first injection. Water was used as a blank injection. The mass spectrometer parameters were as follows: mass range 150-2000 m z; heater temperature at 250° C.; voltage 3.8 kv; sheath gas 40; auxiliary gas 10; capillary temperature 350° C.; and S-lens 50. When mass spectrometry-based identification was not necessary, charged aerosol detection (CAD) was used an analytical flow rate and a desolvation temperature at 100° C. (Lisa et al., “Quantitation of triacylglycerols from plant oils using charged aerosol detection with gradient compensation,” J Chromatogr A. 1176 (1-2): 135-42 (2007); Plante et al., “The use of charged aerosol detection with HPLC for the measurement of lipids,” Methods Mol Biol. 579:469-82 (2009)).

TABLE 2
Chromatography conditions for determination
of polysorbate degradation

| UPLC System | Waters ACQUITY UPLC I-Class/Dionex UltiMate 3000 | | | | |
|---|---|---|---|---|---|
| Mobile Phase | A: 0.1% formic acid in water | | | | |
| | B: 0.1% formic acid in acetonitrile | | | | |
| Column | Waters Oasis ® MAC 30 μm, 2.1 × 20 mm, | | | | |
| | Part No. 186002052 ACQUITY UPLC ® BEH130 | | | | |
| | C4 column, 1.7 μm, 2.1 mm × 50 mm from Waters, | | | | |
| | Part No. 186004496 | | | | |
| Column | 40° C. ± 1° C. | | | | |
| Temperature | | | | | |
| Autosampler | 5° C. ± 2° C. | | | | |
| Temperature | | | | | |
| Injection | 20.0 μL | | | | |
| Volume | | | | | |
| | | | | Flow | |
| | Time (minute) | % A | % B | (μL/minute) | Curve |
| Gradient | Initial | 99.0 | 1.0 | 100 | Initial |
| | 1.0 | 99.0 | 1.0 | 100 | Linear |
| | 5.0 | 85.0 | 15.0 | 100 | Linear |
| | 40.0 | 1.0 | 99.0 | 100 | Linear |
| | 45.0 | 1.0 | 99.0 | 100 | Linear |
| | 45.1 | 99.0 | 1.0 | 100 | Linear |
| | 50.0 | 99.0 | 1.0 | 100 | Linear |

To estimate the total amount of polyoxyethylene (POE), the mass chromatogram was extracted using the 300-800 m/z range to avoid interference from degraded proteins, and the cluster of peaks from about 8-15 minutes was integrated. For CAD chromatograms, the first cluster of POE peaks was directly integrated from about 8-15 minutes (again, retention time may shift slightly). When there were other species co-eluting with the POE, the baseline was adjusted to minimize their impact on the peak area.

To estimate the total amount of POE esters, the mass chromatogram was extracted using the 300-2000 m/z range, and the cluster of peaks from about 17-40 minutes was integrated. For the CAD chromatograms, the POE esters peak cluster was directly integrated from about 17-40 minutes.

Percentage of POE esters was calculated according to the following equation:

$$\frac{POE esters peak area}{POE esters peak area + POE area} \times 100 \%$$

Percentage of remaining POE esters was calculated according to the following equation:

$$\frac{\% POE esters at tn}{\% POE esters at t 0}$$

wherein n=2, 4, or 10 days.

### Stability Testing for Medicinal Products

According to the European Medicines Agency Guideline on Stability Testing of Existing Active Substances and Related Finished Products (CPMP/QWP/122/02, rev 1 corr), active substances stored in long term storage conditions (for example, at 5° C. for active substances intended for storage in a refrigerator, or at 25° C. for active substances intended for storage at room temperature) should be tested every 3 months over the first year, every 6 months over the second year, and annually thereafter through the proposed re-test period. Active substances stored in accelerated storage conditions (for example, at 25° C. for active substances intended for storage in a refrigerator, or at 40° C. for active substances intended for storage at room temperature) should be tested at a minimum of three points, including the initial and final time points, for a 6-month study (for example, testing at 0, 3, and 6 months). An initial 6 months of stability data is required for submission to the EMA.

Similarly, a finished product stored in long term storage conditions (for example, at 5° C. for finished products intended for storage in a refrigerator, or at 25° C. for finished products intended for storage at room temperature) should be tested every 3 months over the first year, every 6 months over the second year, and annually thereafter through the proposed shelf life. A finished product stored in accelerated storage conditions (for example, at 25° C. for finished products intended for storage in a refrigerator, or at 40° C. for finished products intended for storage at room temperature) should be tested at a minimum of three points, including the initial and final time points, for a 6-month study (for example, testing at 0, 3, and 6 months). For conventional dosage forms when the active substances are known to be stable, an initial 6 months of stability data is required for submission to the EMA.

### Example 1. Reduction of Lipase Activity Using Agitation Stress and Heat Stress

In order to reduce esterase and lipase activity, free fatty acid particle formation, and/or polysorbate degradation in therapeutic products, methods for reducing esterase and lipase activity in formulated drug substance were investigated. It was surprisingly discovered that subjecting drug substance to stress conditions, such as agitation stress or heat stress, could be used to reduce esterase and lipase activity and increase the long-term stability of therapeutic molecules and surfactants in drug substance and subsequent drug products. Due to the superior stability of a biotherapeutic compared to an HCP lipase in a drug substance, lipases in a drug substance disproportionately inactivate, degrade and aggregate in response to stress. Therefore, stress conditions can be selected and optimized to provide for the maximum inactivation of lipases while sufficiently preserving the structure and function of a biotherapeutic. Any biotherapeutic that does form a size or charge variant can be removed prior to formulation, for example using CEX or SEC as disclosed in Example 2.

Dupilumab produced from CHO cells, referred to herein as mAb1 drug substance (DS), was subjected to agitation stress. 30 mL of 200 mg/mL mAb1 DS was used. Two 125 mL polycarbonate bottles were each filled with 10 mL of DS and agitated on an orbital shaker at 250 rpm for 0, 24, or 48 hours. The remaining 10 mL of DS were transferred to a 15 mL Falcon tube and served as the unstressed control. 2 mL of each DS was analyzed as either pre-stressed (control) DS (0 hours of agitation) or post-stressed DS (24 or 48 hours of agitation).

The control or stressed DS was used to prepare 150 mg/mL final drug substance (FDS), comprising 150 mg/mL mAb1, 20 mM histidine, 12.5 mM acetate, 5% sucrose, 0.2% high purity PS20, and 25 mM arginine HCl, at pH 5.9. The FDS was filter sterilized and then stored for 0 weeks, 4 weeks, or 8 weeks at 45° C. Storage at 45° C. was selected in order to accelerate the stability study. Physical stability of every sample collected above was analyzed using visual inspection for visible particles and aggregates, SE-UPLC for high and low molecular weight species, micro-flow imaging for subvisible particulate (2-300 μm) analysis, and CAD-UPLC for determining PS20 levels.

mAb1 drug substance subjected to the agitation stress and filtering steps showed less particle formation and greater polysorbate retention over time, as shown in Table 3. For context, the United States Pharmacopeia specificies≤3000 particles of ≥10 μm diameter per container, and ≤ 300 particles of ≥25 um diameter per container, as measured using micro-flow imaging. In the tables in this disclosure, CAD refers to a charged aerosol detector, CEX refers to cation exchange chromatography, FCP refers to a final concentrated pool, HMW refers to high molecular weight species, LMW refers to low molecular weight species, MFI refers to micro-flow imaging, NR indicates a measurement that is not required, OD refers to optical density, RH refers to relative humidity, SE refers to size exclusion, UPLC refers to ultra-performance liquid chromatography, and “-” indicates that no data is available.

TABLE 3
Thermal stability of formulations subjected to agitation stress

| | | Length of Agitation at 250 rpm (hours) | | | | | | | | |
|---|---|---|---|---|---|---|---|---|---|---|
| | | 0 | | | 24 | | | 48 | | |
| | | Length of Storage at 45° C. (weeks) | | | | | | | | |
| | | 0 | 4 | 8 | 0 | 4 | 8 | 0 | 4 | 8 |
| Visual Appearance | | Pass | Pass | Pass | Pass | Pass | Pass | Pass | Pass | Pass |
| Polysorbate Quantification % by CAD | | 100 | 32 | 25 | 100 | 59 | 48 | 100 | 65 | 33 |
| Purity by SE-UPLC | % HMW | 1.7 | — | — | 16.6 | — | — | 30.9 | — | — |
| | % Main | 98.3 | — | — | 83.5 | — | — | 69.1 | — | — |
| | % LMW | 0 | — | — | 0 | — | — | 0 | — | — |
| MFI (# particles/ mL) | 2-10 μm | 1036 | 3260 | 503066 | 739 | 3955 | 7792 | 1269 | 13974 | 14415 |
| | ≥10 μm | 54 | 53 | 81146 | 15 | 53 | 39 | 73 | 140 | 125 |
| | ≥25 μm | 10 | 6 | 17563 | 3 | 3 | 0 | 7 | 14 | 3 |

Additional forms of stress were also investigated. mAb1 drug substance was subjected to heat stress. DS was stored at 45° C. for 0, 0.5, or 1 months in order to cause lipases to degrade, aggregate and/or inactivate. The control and stressed DS were used to prepare 150 mg/mL FDS, comprising 150 mg/mL mAb1, 20 mM histidine, 12.5 mM acetate, 5% sucrose, 0.2% high purity polysorbate 20, and 25 mM arginine HCl, at pH 5.9. The FDS was filter sterilized and then stored under conditions set forth in Table 4.

TABLE 4
Incubation/storage conditions for FDS from heat stressed DS

| Stress Condition | Length of Incubation | |
|---|---|---|
| No storage | t = 0 | |
| 45° C. | 0.5, 1, 1.5, 2 | months |
| 25° C. | 3, 6 | months |
| 5° C. | 3, 6, 12, 18, 24, 36 | months |

Physical stability of the thermally stressed and control non-stressed samples described above was analyzed using visual inspection for visible particles and aggregates, SE-UPLC for high and low molecular weight species, micro-flow imaging for subvisible particulate (2-300 μm) analysis, and CAD-UPLC for determining PS20 levels. Results are set forth below in Tables 5-10. Subjecting DS to thermal stress prior to formulation resulted in a significant decrease of lipase activity, improving polysorbate recovery and decreasing free fatty acid particle formation. Non-stressed samples showed a significant increase in subvisible particles over time that was not observed in stressed samples. Subjecting DS to thermal stress did result in a significant increase in HMW level, but that level remained stable during further incubation.

TABLE 5
Stability of a formulation from DS with 0 months of thermal stress

| | | | Length of Storage at 5° C. (months) | | | | | |
|---|---|---|---|---|---|---|---|---|
| Assay | | t = 0 | 3 | 6 | 12 | 18 | 24 | 36 |
| Visual Appearance | | Pass | Pass | Pass | Pass | Pass | Pass | — |
| Polysorbate Quantification | | 100 | — | 53 | — | — | — | — |
| % by CAD | | | | | | | | |
| Purity by SE- | % HMW | 2.2 | 2.2 | 2.5 | 2.5 | 2.6 | 2.7 | 2.8 |
| UPLC | % Main | 96.6 | 96.6 | 95 | 96.6 | 96.5 | 95.9 | 95.4 |
| | % LMW | 1.2 | 1.2 | 2.5 | 0.9 | 0.9 | 1.5 | 1.8 |
| MFI (# | 2-10 μm | 264 | 12265 | 81,203 | 141583 | 243895 | 273027 | 371554 |
| particles/ mL) | ≥10 μm | 20 | 2273 | 8446 | 16902 | 26739 | 3042 | 38956 |
| | ≥25 μm | 3 | 9 | 47 | 530 | 1780 | 113 | 3602 |

TABLE 6
Stability of a formulation from DS with 0.5 months of thermal stress

| | | | Length of Storage at 5° C. (months) | | | | | |
|---|---|---|---|---|---|---|---|---|
| Assay | | t = 0 | 3 | 6 | 12 | 18 | 24 | 36 |
| Visual Appearance | | Pass | Pass | Pass | Pass | Pass | Pass | — |
| Polysorbate Quantification | | 100 | — | 89 | — | — | — | — |
| % by CAD | | | | | | | | |
| Purity by SE- | % HMW | 10.9 | 10.7 | 11 | 12 | 12 | 11.6 | 11.7 |
| UPLC | % Main | 86.3 | 86.6 | 85.7 | 85.8 | 85.7 | 85.7 | 84.9 |
| | % LMW | 2.8 | 2.7 | 3.4 | 2.2 | 2.3 | 2.7 | 3.4 |
| MFI (# | 2-10 μm | 349 | 6691 | 5828 | 1794 | 3052 | 21842 | 258288 |
| particles/ mL) | ≥10 μm | 16 | 216 | 61 | 34 | 222 | 3641 | 3586 |
| | ≥25 μm | 2 | 21 | 8 | 6 | 36 | 248 | 119 |

TABLE 7
Stability of a formulation from DS with 1 month of thermal stress

| | | | Length of Storage at 5° C. (months) | | | | | |
|---|---|---|---|---|---|---|---|---|
| Assay | | t = 0 | 3 | 6 | 12 | 18 | 24 | 36 |
| Visual Appearance | | Pass | Pass | Pass | Pass | Pass | Pass | — |
| Polysorbate Quantification | | 100 | — | 90 | — | — | — | — |
| % by CAD | | | | | | | | |
| Purity by SE- | % HMW | 17.3 | 17.4 | 17.7 | 19 | 18.9 | 18.6 | 18.5 |
| UPLC | % Main | 78.5 | 78.4 | 77.6 | 77.6 | 77.6 | 77.2 | 76.7 |
| | % LMW | 4.2 | 4.2 | 4.7 | 3.5 | 3.5 | 4.2 | 4.8 |
| MFI (# | 2-10 μm | 160 | 3898 | 2081 | 35220 | 4806 | 824 | 5189 |
| particles/ mL) | ≥10 μm | 7 | 52 | 14 | 1017 | 119 | 39 | 109 |
| | ≥25 μm | 2 | 6 | 1 | 30 | 5 | 2 | 8 |

TABLE 8
Stability of a formulation from DS with 0 months of thermal stress

| | | | Length of Storage at | | | | | |
|---|---|---|---|---|---|---|---|---|
| | | | 25° C. (months) | | Length of Storage at 45° C. (months) | | | |
| Assay | | t = 0 | 3 | 6 | 0.5 | 1 | 1.5 | 2 |
| Visual Appearance | | Pass | Pass | Pass | Pass | — | Pass | — |
| PS-20 level (CAD-UPLC) | | 100 | — | 16 | — | — | — | 33 |
| Purity by SE- | % HMW | 2.2 | 3 | 3.5 | 6.2 | 8.5 | 13.3 | 17.4 |
| UPLC | % Main | 96.6 | 95.8 | 94.2 | 92.2 | 89.2 | 84.4 | 79.9 |
| | % LMW | 1.2 | 1.2 | 2.3 | 1.6 | 2.3 | 2.3 | 2.7 |
| MFI (# | 2-10 μm | 264 | 19609 | 146335 | 25146 | 2532 | 8226 | 263410 |
| particles/ mL) | ≥10 μm | 20 | 492 | 23326 | 750 | 47 | 206 | 41033 |
| | ≥25 μm | 3 | 19 | 3825 | 60 | 5 | 20 | 9790 |

TABLE 9
Stability of a formulation from DS with 0.5 months of thermal stress

| | | | Length of Storage at | | | | | |
|---|---|---|---|---|---|---|---|---|
| | | | 25° C. (months) | | Length of Storage at 45° C. (months) | | | |
| Assay | | t = 0 | 3 | 6 | 0.5 | 1 | 1.5 | 2 |
| Visual Appearance | | Pass | Pass | Pass | Pass | — | Pass | — |
| PS-20 level (CAD-UPLC) | | 100 | — | 68 | — | — | — | 75 |
| Purity by SE- | % HMW | 10.9 | 11 | 11.5 | 14.5 | 14.6 | 21.2 | 24.8 |
| UPLC | % Main | 86.3 | 86.2 | 84.4 | 82.3 | 81 | 74.5 | 70.3 |
| | % LMW | 2.8 | 2.8 | 4.2 | 3.2 | 4.4 | 4.4 | 4.9 |
| MFI (# | 2-10 μm | 349 | 6232 | 8660 | 4838 | 9249 | 18683 | 7797 |
| particles/ mL) | ≥10 μm | 16 | 52 | 169 | 60 | 172 | 426 | 272 |
| | ≥25 μm | 2 | 4 | 3 | 8 | 0 | 29 | 6 |

TABLE 10
Stability of a formulation from DS with 1 month of thermal stress

| | | | Length of Storage at | | | | | |
|---|---|---|---|---|---|---|---|---|
| | | | 25° C. (months) | | Length of Storage at 45° C. (months) | | | |
| Assay | | t = 0 | 3 | 6 | 0.5 | 1 | 1.5 | 2 |
| Visual Appearance | | Pass | Pass | Pass | Pass | — | Pass | — |
| PS-20 level (CAD-UPLC) | | 100 | — | 77 | — | — | — | 84 |
| Purity by SE- | % HMW | 17.3 | 17.5 | 17.6 | 20.8 | 19.4 | 27 | 30.2 |
| UPLC | % Main | 78.5 | 78.4 | 77.2 | 74.6 | 74.4 | 67.4 | 63.6 |
| | % LMW | 4.2 | 4.2 | 5.2 | 4.6 | 6.2 | 5.6 | 6.3 |
| MFI (# | 2-10 μm | 160 | 5799 | 11893 | 3258 | 10647 | 18440 | 4921 |
| particles/ mL) | ≥10 μm | 7 | 84 | 236 | 38 | 359 | 453 | 70 |
| | ≥25 μm | 2 | 3 | 19 | 10 | 18 | 28 | 4 |

### Example 2. Depletion of High Molecular Weight Species from Stress Pre-Treated Drug Substance

As disclosed in Example 1, subjecting a drug substance to agitation stress or thermal stress can result in an increase in HMW species. Compare, for example, the 2.2% HMW species at time zero for untreated DS in Tables 5 and 8, compared to 10.9% for DS treated to 0.5 months of thermal stress in Tables 6 and 9, and 17.3% for DS treated to 1 month of thermal stress in Tables 7 and 10. HMW species may be formed from HCPs, drug protein, or a combination. HMW species can be removed using further processing steps such as filtration or chromatography.

mAb1 DS was subjected to 1 or 2 weeks of thermal stress at 45° C. or 50° C. Thermally stressed DS was subjected to cation exchange (CEX) chromatography to remove HMW species that formed during stress conditions. HMW species were efficiently depleted from stressed DS, as shown in Table 11. The HMW-depleted drug substance samples had a comparable percent of HMW species (2.3% or 2.5%) compared to a drug substance that was not subjected to stress or purification (1.7%). The percentage of HMW species was reduced to an acceptable level for a pharmaceutical composition, in particular below 5%.

TABLE 11
Clearance of HMW species from stressed drug substance

| | | | Thermal stress of 1 | | Thermal stress of 2 | |
|---|---|---|---|---|---|---|
| | | | week | | weeks | |
| Assay | | t = 0 | 45° C. | 50° C. | 45° C. | 50° C. |
| Purity by SE-UPLC before | % HMW | 1.7 | 10.2 | 26.2 | 17.0 | 33.6 |
| CEX purification | % Main | 98.3 | 89.8 | 73.8 | 83.0 | 66.4 |
| Purity by SE-UPLC after | % HMW | 1.3 | 2.3 | 2.3 | 2.5 | 2.5 |
| CEX purification | % Main | 98.7 | 97.7 | 97.7 | 97.5 | 97.5 |

HMW-depleted DS was formulated into FDS comprising 150 mg/mL mAb1, 20 mM histidine, 12.5 mM acetate, 5% sucrose, 0.2% PS80, and 25 mM arginine HCl, at pH 5.9. The stability of FDS from HMW-depleted thermally stressed DS was compared to FDS from HMW-depleted non-stressed DS. In particular, non-stressed DS (Table 12) was compared with DS stressed at 45° C. for 1 week (Table 13), 50° C. for 1 week (Table 14), 45° C. for 2 weeks (Table 15), and 50° C. for 2 weeks (Table 16). Each FDS was stored at 1, 3, or 6 months at 25° C., or 1 week, 2 weeks, 1 month, or 3 months at 45° C. before analysis. Visual appearance, turbidity, pH, PS80 recovery, purity as measured by SE-UPLC, charge variants, mAb1 recovery, number of particles/mL≥10 μm in size, and number of particles/mL≥25 μm in size were characterized.

As shown in Table 12, the FDS from control DS shows a substantial loss in PS80 after 3 months of storage, down to 50% after storage at 25° C. and 47% after storage at 45° C., illustrating the significance of lipase activity in the FDS sample. A loss of 50% of PS80 would typically be considered unacceptable for a pharmaceutical composition. Accordingly, the number of particles/mL counted by microflow imaging also increased greatly over time, to as high as 815 particles/mL≥10 μm after 3 months of storage at 25° C. PS80 degradation continued after 6 months of storage at 25° C., with the percent of PS80 recovered decreasing to 43%. The number of particles/mL measured remained high. It should be noted that, because particles may form and break apart dynamically, it is not surprising that the particle count does not linearly increase, while remaining high from 3 to 6 months.

As shown in Table 13, the FDS from DS pre-treated at 45° C. for 1 week shows a substantially different profile. After 3 months of storage, PS80 levels remained at 90% at 25° C. and 85% at 45° C., and remained at 83% after 6 months at 25° C. A loss of less than 20% of PS80 may be considered acceptable for a pharmaceutical composition. The number of particles/mL remained low after 3 months and 6 months of storage, only increasing to 21 particles/mL≥10 μm and 2 particles/mL≥25 μm after 3 months of storage at 45° C., which is within the range of background noise for this assay (below about 100). Therefore, even the mildest pre-treatment condition showed marked improvement compared to the non-stressed control in terms of polysorbate retention, reduction of lipase activity, and reduction of particle formation.

As shown in Table 14, the FDS from DS pre-treated at 50° C. for 1 week shows even higher PS80 stability. About 96% and 94% of PS80 was recovered after 3 months of storage at 25° C. and 45° C., respectively, and about 98% after 6 months of storage at 25° C. Given a margin of error of about 10% using this assay, this is statistically indistinguishable from full recovery at all time points and storage conditions. The number of particles/mL remained low, within the range of background noise. Overall, the stability of the FDS from DS pre-treated at 50° C. for 1 week is similar to what would be expected in an FDS with no lipase contamination at all.

As shown in Table 15, the FDS from DS pre-treated at 45° C. for 2 weeks shows improved polysorbate stability similar to that seen from the pre-treatment at 45° C. for 1 week condition, with a recovery of PS80 above 90% even after 6 months. Particle data was not collected from the samples pre-treated with heat stress for 2 weeks due to a low amount of sample remaining.

As shown in Table 16, the FDS from DS pre-treated to 50° C. for 2 weeks shows effectively no lipase activity and complete polysorbate stability, with 100% of PS80 measured as recovered after 3 months of storage at 25° C., and 97% (within measurement error of 100%) after 6 months.

mAb1 stability (in terms of size variants and charge variants) and recovery were not significantly impacted by the DS treatment over the time frame of these experiments, as was expected over a short time frame. However, the dramatic loss of polysorbate seen in the control condition (Table 12) may be expected to result in a subsequent loss of mAb1 stability over the course of long-term storage.

It should be noted that the CEX step following stress pre-treatment may also deplete remaining HCP lipases from each of the samples in addition to non-functional HMW species. This feature may be masking an even greater difference in lipase activity between stressed and non-stressed DS attributable to thermal stress alone.

TABLE 12
Thermal stability of a formulation from HMW-depleted control DS

| | | | Length of Storage at | | | | | | |
|---|---|---|---|---|---|---|---|---|---|
| | | | 25° C. (months) | | | Length of Storage at 45° C. (months) | | | |
| Assay | | t = 0 | 1 | 3 | 6 | 0.25 | 0.5 | 1 | 3 |
| Visual Appearance | | Pass | Pass | Pass | Pass | Pass | Pass | Pass | Pass |
| Increase in Turbidity (OD 405 nm) | | 0.00 | 0.01 | 0.03 | 0.08 | 0.03 | 0.07 | 0.09 | 0.29 |
| pH | | 6.2 | 6.3 | 6.2 | 6.2 | 6.2 | 6.1 | 6.3 | 6.2 |
| PS-80 level (CAD-UPLC) | | 100 | 68 | 50 | 43 | 68 | 59 | 59 | 47 |
| Purity by SE-UPLC | % HMW | 2.2 | 2.7 | 3.0 | 3.8 | 4.0 | 4.9 | 7.3 | 24.5 |
| | % Main | 97.7 | 97.1 | 95.6 | 95.6 | 95.7 | 94.5 | 92.2 | 64.8 |
| | % LMW | 0.2 | 0.2 | 1.4 | 0.6 | 0.3 | 0.6 | 0.4 | 10.7 |
| Charge Variant | % Acidic | 26.9 | 29.5 | 32.8 | 37.6 | 34.5 | 46.9 | 50.2 | 59.5 |
| Analysis by CEX- | % Main | 72.8 | 68.8 | 64.9 | 59.4 | 61.6 | 49.3 | 48.4 | 39.6 |
| UPLC | % Basic | 0.3 | 1.7 | 2.3 | 3.0 | 4.0 | 3.9 | 1.4 | 0.9 |
| % mAb1 recovered by RP-UPLC | | 100 | 103 | 99 | 101 | 101 | 98 | 99 | 97 |
| MFI (# particles/mL) | ≥10 μm | 38 | 13 | 815 | 108 | 31 | 13 | 10 | 696 |
| | ≥25 μm | 2 | 2 | 2 | 8 | 4 | 7 | 4 | 42 |

TABLE 13
Thermal stability of a formulation from HMW-depleted DS pre-treated at 45° C. for 1 week

| | | | Length of Storage at | | | | | | |
|---|---|---|---|---|---|---|---|---|---|
| | | | 25° C. (months) | | | Length of Storage at 45° C. (months) | | | |
| Assay | | t = 0 | 1 | 3 | 6 | 0.25 | 0.5 | 1 | 3 |
| Visual Appearance | | Pass | Pass | Pass | Pass | Pass | Pass | Pass | Pass |
| Increase in Turbidity (OD 405 nm) | | 0.00 | 0.00 | 0.02 | 0.06 | 0.01 | 0.05 | 0.07 | 0.27 |
| pH | | 6.2 | 6.3 | 6.2 | 6.2 | 6.2 | 6.2 | 6.3 | 6.2 |
| PS-80 level (CAD-UPLC) | | 100 | 98 | 90 | 83 | 97 | 86 | 94 | 85 |
| Purity by SE-UPLC | % HMW | 2.7 | 3.0 | 3.4 | 3.9 | 3.8 | 4.7 | 6.5 | 21.5 |
| | % Main | 97.0 | 96.6 | 95.4 | 95.2 | 95.9 | 94.4 | 92.9 | 56.6 |
| | % LMW | 0.3 | 0.4 | 1.2 | 0.9 | 0.3 | 1.0 | 0.6 | 21.9 |
| Charge Variant | % Acidic | 18.0 | 34.1 | 38.0 | 41.7 | 38.8 | 48.0 | 53.9 | 65.6 |
| Analysis by CEX- | % Main | 76.3 | 60.9 | 60.0 | 56.8 | 56.2 | 47.9 | 44.7 | 33.8 |
| UPLC | % Basic | 5.7 | 5.0 | 2.0 | 1.5 | 5.0 | 4.2 | 1.4 | 0.7 |
| % mAbl recovered by RP-UPLC | | 100 | 102 | 99 | 102 | 103 | 100 | 99 | 98 |
| MFI (# particles/mL) | ≥10 μm | 2 | 10 | 2 | 19 | 23 | 8 | 10 | 21 |
| | ≥25 μm | 0 | 2 | 2 | 0 | 2 | 0 | 2 | 2 |

TABLE 14
Thermal stability of a formulation from HMW-depleted DS pre-treated at 50° C. for 1 week

| | | | Length of Storage at | | | | | | |
|---|---|---|---|---|---|---|---|---|---|
| | | | 25° C. (months) | | | Length of Storage at 45° C. (months) | | | |
| Assay | | t = 0 | 1 | 3 | 6 | 0.25 | 0.5 | 1 | 3 |
| Visual Appearance | | Pass | Pass | Pass | Pass | Pass | Pass | Pass | Pass |
| Increase in Turbidity (OD 405 nm) | | 0.00 | 0.0 | 0.01 | 0.04 | 0.0 | 0.03 | 0.03 | 0.14 |
| pH | | 6.1 | 6.1 | 6.0 | 6.1 | 6.0 | 6.0 | 6.1 | 6.0 |
| PS-80 level (CAD-UPLC) | | 100 | 101 | 96 | 98 | 101 | 93 | 101 | 94 |
| Purity by SE-UPLC | % HMW | 3.0 | 3.3 | 3.5 | 4.1 | 4.0 | 4.3 | 6.2 | 18.9 |
| | % Main | 96.7 | 96.2 | 95.3 | 95.0 | 95.7 | 94.6 | 93.2 | 70.4 |
| | % LMW | 0.3 | 0.5 | 1.2 | 0.9 | 0.3 | 1.1 | 0.6 | 10.7 |
| Charge Variant | % Acidic | 30.5 | 36.1 | 38.7 | 42.2 | 41.0 | 53.9 | 54.8 | 60.1 |
| Analysis by CEX- | % Main | 62.8 | 61.1 | 59.3 | 56.3 | 53.7 | 43.7 | 43.5 | 38.9 |
| UPLC | % Basic | 6.7 | 2.8 | 2.0 | 1.4 | 5.4 | 2.3 | 1.8 | 1.1 |
| % mAb1 recovered by RP-UPLC | | 100 | 102 | 99 | 101 | 103 | 95 | 99 | 97 |
| MFI (# particles/mL) | ≥10 μm | 27 | 21 | 35 | 77 | — | 19 | 13 | 2 |
| | ≥25 μm | 8 | 4 | 6 | 27 | — | 4 | 2 | 0 |

TABLE 15
Thermal stability of a formulation from HMW-depleted DS pre-treated at 45° C. for 2 weeks

| | | | Length of Storage at 25° C. | | | | | | |
|---|---|---|---|---|---|---|---|---|---|
| | | | (months) | | | Length of Storage at 45° C. (months) | | | |
| Assay | | t = 0 | 1 | 3 | 6 | 0.25 | 0.5 | 1 | 3 |
| Visual Appearance | | Pass | Pass | Pass | Pass | Pass | Pass | Pass | Pass |
| Increase in Turbidity (OD 405 nm) | | 0.00 | 0.02 | 0.03 | 0.03 | 0.03 | 0.01 | 0.01 | 0.24 |
| pH | | 6.1 | 6.2 | 6.1 | 6.2 | 6.1 | 61. | 6.1 | 6.2 |
| PS-80 level (CAD-UPLC) | | 100 | 92 | 96 | 92 | 91 | 95 | 88 | 91 |
| Purity by SE-UPLC | % HMW | 2.9 | 3.1 | 3.4 | 3.9 | 5.5 | 4.1 | 9.5 | 19.2 |
| | % Main | 96.5 | 96.0 | 95.3 | 95.1 | 94.1 | 95.0 | 86.2 | 71.7 |
| | % LMW | 0.6 | 0.9 | 1.4 | 1.0 | 0.4 | 0.9 | 4.4 | 9.2 |
| Charge Variant | % Acidic | 36.4 | 38.9 | 41.6 | 45.7 | 59.1 | 46.0 | 58.8 | 58.8 |
| Analysis by CEX- | % Main | 56.4 | 58.7 | 56.4 | 51.7 | 39.3 | 52.3 | 39.7 | 39.7 |
| UPLC | % Basic | 7.3 | 2.4 | 2.1 | 2.6 | 1.6 | 1.7 | 1.5 | 1.5 |

| | | | | | | | | |
|---|---|---|---|---|---|---|---|---|
| % mAb1 recovered by RP-UPLC | 100 | 98 | 99 | 102 | 107 | 103 | 101 | 99 |

TABLE 16
Thermal stability of a formulation from HMW-depleted DS pre-treated at 50° C. for 2 weeks

| | | | Length of Storage at 25° C. | | | | | | |
|---|---|---|---|---|---|---|---|---|---|
| | | | (months) | | | Length of Storage at 45° C. (months) | | | |
| Assay | | t = 0 | 1 | 3 | 6 | 0.25 | 0.5 | 1 | 3 |
| Visual Appearance | | Pass | Pass | Pass | Pass | Pass | Pass | Pass | Pass |
| Increase in Turbidity (OD 405 nm) | | 0.00 | 0.09 | 0.03 | 0.03 | 0.03 | 0.01 | 0.07 | 0.22 |
| pH | | 6.0 | 6.2 | 6.1 | 6.2 | 6.1 | 6.1 | 6.1 | 6.1 |
| PS-80 level (CAD-UPLC) | | 100 | 94 | 100 | 97 | 91 | 99 | 94 | 95 |
| Purity by SE-UPLC | % HMW | 2.8 | 3.0 | 3.1 | 3.7 | 5.1 | 3.9 | 8.2 | 17.8 |
| | % Main | 96.5 | 96.1 | 95.3 | 95.0 | 94.4 | 95.1 | 85.9 | 69.6 |
| | % LMW | 0.7 | 1.0 | 1.7 | 1.3 | 0.5 | 1.0 | 5.9 | 12.6 |
| Charge Variant | % Acidic | 42.4 | 42.6 | 47.2 | 50.7 | 43.8 | 51.0 | 60.9 | 65.2 |
| Analysis by CEX- | % Main | 50.6 | 54.7 | 51.1 | 46.6 | 53.9 | 46.8 | 35.3 | 33.8 |
| UPLC | % Basic | 7.0 | 2.7 | 1.7 | 2.7 | 2.3 | 2.2 | 3.8 | 1.0 |

| | | | | | | | | |
|---|---|---|---|---|---|---|---|---|
| % mAb1 recovered by RP-UPLC | 100 | 97 | 99 | 101 | 119 | 104 | 101 | 100 |

These results demonstrate that the disclosed methods of subjecting drug substance to agitation stress or heat stress, optionally followed by HMW depletion, produce an improved pharmaceutical composition with reduced lipase activity, reduced polysorbate degradation, and reduced free fatty acid particle formation.

The present invention is not to be limited in scope by the specific aspects described herein. Indeed, various modifications of the invention in addition to those described herein will become apparent to those skilled in the art from the foregoing description. Such modifications are intended to fall within the scope of the appended claims.

What is claimed is:

1. A method for producing a pharmaceutical composition with reduced lipase activity, comprising:
(a) subjecting a sample including a protein of interest and a lipase to stress conditions to form a sample with inactivated lipase;
(b) subjecting said sample with inactivated lipase to a purification step to form a high molecular weight (HMW)-depleted sample; and
(c) formulating said HMW-depleted sample with at least one fatty acid ester to produce a pharmaceutical composition with reduced lipase activity,
wherein said pharmaceutical composition has reduced lipase activity compared to a pharmaceutical composition formulated from a sample that was not subjected to stress conditions, and wherein said reduced lipase activity is measured after a period of storage at long-term storage conditions or accelerated storage conditions of at least 2 weeks.

2. The method of claim 1, wherein said protein of interest is an antibody, an antibody-derived protein, an antibody fragment, a monoclonal antibody, a bispecific antibody, a fusion protein, an antibody-drug conjugate, or a therapeutic protein.

3. The method of claim 1, wherein said protein of interest is Dupilumab.

4. The method of claim 2, wherein said stress conditions include agitation stress and/or heat stress.

5. The method of claim 4, wherein said agitation stress comprises shaking said sample at from 50 to 500 rpm, from 200 to 300 rpm, about 50 rpm, about 75 rpm, about 100 rpm, about 125 rpm, about 150 rpm, about 200 rpm, about 225 rpm, about 250 rpm, about 275 rpm, about 300 rpm, about 325 rpm, about 350 rpm, about 375 rpm, about 400 rpm, about 425 rpm, about 450 rpm, about 475 rpm, or about 500 rpm.

6. The method of claim 5, wherein said agitation stress comprises shaking said sample for from 1 to 96 hours, from 24 to 48 hours, about 1 hour, about 2 hours, about 3 hours, about 4 hours, about 5 hours, about 6 hours, about 12 hours, about 18 hours, about 24 hours, about 30 hours, about 36 hours, about 42 hours, about 48 hours, about 60 hours, about 72 hours, about 84 hours, or about 96 hours.

7. The method of claim 4, wherein said heat stress comprises storing said sample at from about 25° C. to about 60° C., from about 30° C. to about 60° C., from about 35° C. to about 55° C., from about 40° C. to about 50° C., from about 44° C. to about 46° C., about 25° C., about 30° C., about 35° C., about 40° C., about 45° C., about 50° C., about 55° C., or about 60° C.

8. The method of claim 7, wherein said heat stress comprises storing said sample at said temperature from 1 day to 6 months, from 3 days to 3 months, from 1 week to 2 months, from 0.5 months to 1 month, about 1 day, about 2 days, about 3 days, about 1 week, about 2 weeks, about 0.5 months, about 3 weeks, about 4 weeks, about 1 month, about 5 weeks, about 6 weeks, about 7 weeks, about 8 weeks, about 2 months, about 3 months, about 3.5 months, about 4 months, about 4.5 months, about 5 months, about 5.5 months, or about 6 months.

9. The method of claim 4, wherein said purification step comprises filtration, enrichment, or chromatographic separation.

10. The method of claim 9, wherein said chromatographic separation comprises cation exchange chromatography.

11. The method of claim 9, wherein said chromatographic separation comprises size exclusion chromatography.

12. The method of claim 9, wherein said HMW-depleted sample comprises less than 10%, less than 5%, less than 4%, less than 3%, less than 2%, or less than 1% HMW species.

13. The method of claim 9, wherein said formulating step further comprises adding excipients to said HMW-depleted sample.

14. The method of claim 9, wherein said fatty acid ester is polysorbate 20, polysorbate 40, polysorbate 60, or polysorbate 80.

15. The method of claim 9, wherein said period of storage is from about 2 weeks to about 5 years, from about 2 weeks to about 3 years, from about 2 weeks to about 1 year, from about 2 weeks to about 6 months, from about 1 month to about 5 years, from about 1 month to about 3 years, from about 1 month to about 1 year, from about 1 month to about 6 months, about 2 weeks, about 3 weeks, about 4 weeks, about 1 month, about 2 months, about 3 months, about 4 months, about 5 months, about 6 months, about 9 months, about 12 months, about 18 months, about 2 years, about 3 years, about 4 years, or about 5 years.

16. The method of claim 4, wherein said long-term storage conditions comprise storage at a temperature from about 0° C. to about 10° C., about 0° C., about 5° C., or about 10° C.

17. The method of claim 4, wherein said accelerated storage conditions comprise storage at a temperature from about 35° C. to about 60° C., from about 45° C. to about 55° C., about 35° C., about 40° C., about 45° C., about 50° C., about 55° C., or about 60° C.

18. The method of claim 4, wherein a measure of reduced lipase activity comprises a quantification of fatty acid ester degradation.

19. The method of claim 18, wherein said quantification comprises an enzymatic colorimetric assay for non-esterified fatty acids, mass spectrometry, charged aerosol detection, and/or liquid chromatography.

20. The method of claim 18, wherein said quantification comprises determining a percent of fatty acid ester recovered from said pharmaceutical composition with reduced lipase activity after said period of storage using charged aerosol detection-liquid chromatography.

21. The method of claim 20, wherein said percent is above 80%, above 85%, above 90%, above 95%, above 99%, about 85%, about 90%, about 95%, about 99%, or about 100%.

22. The method of claim 4, wherein a measure of reduced lipase activity comprises a quantification of particles per unit volume.

23. The method of claim 22, wherein said particles comprise visible particles and/or subvisible particles.

24. The method of claim 22, wherein said particles comprise particles≥10 μm in diameter and/or particles≥25 um in diameter.

25. The method of claim 22, wherein said particles comprise fatty acid particles.

26. The method of claim 22, wherein said quantification comprises light obscuration, optical microscopy, micro-flow image analysis, flow cytometry, Coulter counting, and/or submicron particle tracking.

27. The method of claim 22, wherein a number of particles per mL in said pharmaceutical composition with reduced lipase activity is below 500, below 200, below 100, below 50, below 10, or below 5.

28. The method of claim 4, wherein said recombinant host cell is selected from a group consisting of a CHO cell, a CHO-K1 cell, and variations thereof.

29. The method of claim 27, wherein said lipase is a host cell protein.

30. The method of claim 29, wherein said lipase has an increased susceptibility to inactivation in stress conditions compared to said protein of interest.

31. A protein formulation comprising a protein and at least one fatty acid ester made by the method of claim 4.
